(19) United States
(12) Patent Application Publication
(10) Pub. No.: US 2024/0410004 A1
(43) Pub. Date: Dec. 12, 2024

(54) A METHOD FOR DIAGNOSING AND PREDICTING PROGRESSION OF NEURODEGENERATIVE DISEASES OR DISORDERS

(71) Applicant: TIVENIX SA, Lugano (CH)

(72) Inventor: Luca COLNAGHI, Cornate d' Adda (IT)

(21) Appl. No.: 18/697,252

(22) PCT Filed: Sep. 30, 2022
(86) PCT No.: PCT/EP2022/077418

§ 371 Date: Mar. 29, 2024

(30) Foreign Application Priority Data
Sep. 30, 2021 (EP) 21200177.0

(51) Int. Cl. C12Q 1/6883

(52) U.S. Cl. CPC C12Q 1/6883; C12Q 2600/154

(57) ABSTRACT

The invention relates to a method for determining a score indicative of the diagnosis of a neurodegenerative disease or disorder of a subject, of the probability of a subject to develop a neurodegenerative disease or disorder and/or of the disease progression of a neurodegenerative disease or disorder that is based on comparing a methylation status to a reference pattern or comparing a methylation status and further markers to a reference pattern. The methylation status may be derived from cell free DNA e.g. in a plasma sample. The reference pattern can be embodied in a library, or on a storage device and may be obtained from reference subjects e.g. by a machine-learning technique. The invention further relates to a method for monitoring a neurodegenerative disease or disorder. The neurodegenerative disease or disorder may for example be Alzheimer's disease or Parkinson's disease.

The invention relates to a method for determining a score indicative of the diagnosis of a neurodegenerative disease or disorder of a subject, of the probability of a subject to develop a neurodegenerative disease or disorder and/or of the disease progression of a neurodegenerative disease or disorder that is based on comparing a methylation status to a reference pattern or comparing a methylation status and further markers to a reference pattern. The methylation status may be derived from cell free DNA e.g. in a plasma sample. The reference pattern can be embodied in a library, or on a storage device and may be obtained from reference subjects e.g. by a machine-learning technique. The invention further relates to a method for monitoring a neurodegenerative disease or disorder. The neurodegenerative disease or disorder may for example be Alzheimer's disease or Parkinson's disease.

Neurodegenerative diseases and disorders are a major reason for morbidity and mortality for the aging society, with no cure yet available. For example, at least 40 million people worldwide suffer from dementia and their number may increase to 120 million in the next 30 years (Alzheimer's disease facts and figures, 2020, Alzheimer's & Dementia 16, 391-460). There is thus a strong need to understand better the etiology and pathogenesis of neurodegenerative diseases and disorders. Dementia embraces multifactorial neurodegenerative disorders caused by a combination of genetic and environmental factors. Alzheimer's disease (AD) is the most prevalent type of dementia and a leading cause of death. The pathogenic process leading to dementia may start decades before the onset of first clinical symptoms for which unbiased, quantitative and unequivocal measures are missing (Weller, J. & Budson, A., 2018, F1000Res 7). AD is clinically characterized by the progressive impairment of memory and cognitive functions accompanied by abnormal protein deposition and neuronal loss. Neuropathological changes in AD begin with the extracellular deposition of β-amyloid peptides in senile plaques decades before the appearance of first clinical symptoms (Makin, S., 2018, Nature 559, S4-S4). Aberrant β-amyloid peptides may facilitate the accumulation of hyperphosphorylated and fibrillar Tau protein in intraneuronal neurofibrillary tangles, suggesting that the two disease hallmarks senile plaques and neurofibrillary tangles may cooperate in the progression of AD (Kametani, F. & Hasegawa, M., 2018, Front. Neurosci. 12). Neurofibrillary tangle deposition in the AD brain matches with neuronal loss and severity of cognitive impairments, and follows a stereotypic pattern beginning in the entorhinal cortex, progressing to the hippocampus, and then invading the frontal, temporal, and parietal cortex (Schultz, S. A. et al., 2018, Neurobiology of Aging 72, 177-185).

Neurodegenerative diseases and disorders are not a normal manifestation of aging and the disease causes remain unclear. When AD occurs before 65 years of age it is classified as early-onset AD and in most cases it is an expression of an autosomal dominant genetic competent, such as heritable mutations in the genes encoding for the amyloid precursor protein or the presenilins, which cause familial AD (Desikan, R. S. et al., 2017, PLOS Medicine 14, e1002258; Bertram, L, Lill, C. M. & Tanzi, R. E., 2010, Neuron 68, 270-281). Late onset AD occurs in over 90% of the cases, whereby a clear genetic association is missing (sporadic AD), suggesting that environmental and lifestyle factors may negatively influence the genetic program of the cell. This epigenetic regulation encompasses modification of histones, other regulatory proteins, non-coding RNAs and DNA.

Clinical diagnosis of AD reaches an accuracy of maximal 75% (Hansson O. Nat Med. 2021 June; 27(6):954-963) and is thus often improved to 90-92% when complemented by measuring β-amyloid peptides (Aβ42, Aβ40, ratio Aβ42/Aβ40, and phospho-Tau (pTau181) in cerebrospinal fluid (El Kadmiri, N., et al., 2018, S. Neuroscience 370, 181-190; Blennow, K., Hampel, H., Weiner, M. & Zetterberg, H., 2010, Nature Reviews Neurology 6, 131-144). Amyloid or Tau positron emission tomography (PET) is an emerging technology to detect protein deposition in the brain and represent an instrumental solution for the diagnosis of AD (Lemoine, L, et al., 2018, Alzheimer's & Dementia: Diagnosis, Assessment & Disease Monitoring 10, 232-236; 8. Palmqvist, S. et al., 2014, JAMA NeurologyTi, 1282-1289); however it is costly and rarely used before first signs of cognitive decline. Changes relating to a disease-unspecific neurodegenerative process are detected as atrophy of specific brain regions using for example MRI, and/or as an increase in total Tau or neurofilament light chain released from the dying neuron in the cerebrospinal fluid. These IVD (in vitro diagnostic) markers exhibit unsatisfying specificity at an early stage of dementia and require a relatively invasive intervention. Emerging IVD blood markers under investigation are the ratio Aβ42/Aβ40, phospho-Tau (pTau217), the neurofilament light chain or the glial fibrillary acidic protein (GFAP) revealing an ongoing neuroinflammation.

Thus, there is a need for an improved method for diagnosing and/or predicting progression of neurodegenerative diseases or disorders and/or distinguishing among different diseases or disorders.

The above technical problem is solved by the embodiments disclosed herein and as defined in the claims.

Accordingly, the invention relates to, inter alia, the following embodiments:

1. A method for determining a score indicative of the diagnosis of a neurodegenerative disease or disorder of a subject, of the probability of a subject to develop a neurodegenerative disease or disorder and/or of the disease progression of a neurodegenerative disease or disorder, the method comprising the steps of:
a) obtaining at least one methylation status of two or more genes selected from Table 1 and/or two or more regions selected from Table 2 from a sample of a subject;
b) comparing the methylation status obtained in (a) to a reference pattern; and
c) determining a score indicative of the diagnosis of a neurodegenerative disease or disorder of the subject, of the probability of the subject to develop a neurodegenerative disease or disorder and/or of the disease progression of a neurodegenerative disease or disorder based on the comparison obtained in (b).
   2. The method of embodiment 1, wherein the sample is a body fluid sample preferably a plasma or serum sample, more preferably a plasma sample.
   3. The method of embodiment 2, wherein the sample is or was frozen.
   4. The method of any one of embodiments 1 to 3, wherein the methylation status is obtained from cell free-DNA in the sample.
   5. The method of any one of embodiments 1 to 4, wherein obtaining at least one methylation status comprises genome methylation profiling.
   6. The method of any one of embodiments 1 to 5, wherein the at least one methylation status comprises the methylation status of at least 100 regions selected from Table 2, preferably at least 150 regions selected from Table 2, preferably at least 200 regions selected from Table 2, more preferably between 200 and 600 regions selected from Table 2, more preferably 250 and 500, more preferably between 250 and 300 regions selected from Table 2.
   7. The method of embodiment 6, wherein the at least one methylation status comprises the methylation status of at least 80% of the regions selected from Table 3, preferably 90% of the regions selected from Table 3, more preferably all regions selected from Table 3.
   8. The method of any of the previous embodiments, wherein the methylation status is determined in regions with
a) an average nucleotide width of less than 5000, preferably less than 4000, more preferably less than 3000, more preferably less than 2000, more preferably less than 1000; and/or
b) a median nucleotide width of less than 5000, preferably less than 4000, more preferably less than 3000, more preferably less than 2000, more preferably less than 1000.9. The method of any one of embodiments 1 to 8, wherein at least one further marker is obtained in (a) compared in (b).
   10. The method of embodiment 9, wherein the at least one further marker is a marker selected from the group of subject background data, cognitive performance marker, autonomic nervous system biomarker.
   11. The method of any one of embodiments 1 to 10, wherein the reference pattern is obtained from the methylation status of two or more genes selected from Table 1 and/or two or more regions selected from Table 2 from samples of at least two reference subjects, wherein at least one of the reference subjects suffers from a neurodegenerative disease or disorder.
   12. The method of embodiment 10 or 11, wherein obtaining the reference pattern from the methylation status of two or more genes selected from Table 1 and/or two or more regions selected from Table 2 from samples of at least two reference subjects comprises a machine learning technique.
   13. A method for monitoring a neurodegenerative disease or disorder, the method comprising the steps of:
i) determining a first score indicative of the probability of a subject to develop a neurodegenerative disease or disorder and/or of the disease progression of a neurodegenerative disease or disorder according to the method of any one of embodiments 1 to 12 at a first timepoint;
ii) determining a second score indicative of the probability of a subject to develop a neurodegenerative disease or disorder and/or of the disease progression of a neurodegenerative disease or disorder according to the method of any one of embodiments 1 to 12 at a second timepoint;
iii) comparing the first score of step (i) with the second score of step (ii); and
iv) monitoring the disease progression of the neurodegenerative disease or disorder in the subject based on the comparison of step (iii).
   14. A library comprising at least one score indicative of the probability of a subject to develop a neurodegenerative disease or disorder and/or of the disease progression of a neurodegenerative disease or disorder determined according to any one of the embodiments 1 to 12.
   15. A storage device comprising computer-readable program instructions to execute the method according to any one of the embodiments 1 to 12, preferably additionally comprising the library of embodiment 14.
   16. A server comprising the storage device of embodiment 15, at least one processing device, and a network connection for receiving data indicative of at least one methylation status.
   17. The method of any one of embodiments 1 to 13, the library of embodiment 14, the storage device of embodiment 15, the server of embodiment 16, wherein the neurodegenerative disease or disorder is a disease or disorder characterized by cognitive impairment.
   18. The method of any one of embodiments 1 to 13, the library of embodiment 14, the storage device of embodiment 15, the server of embodiment 16, wherein the neurodegenerative disease or disorder is Alzheimer's disease and/or Parkinson's disease.
   19. The method of embodiment 18, the library of embodiment 18, the storage device of embodiment 18, the server of embodiment 18, wherein the neurodegenerative disease or disorder is Alzheimer's disease.
   20. A device comprising methylation specific oligonucleotide probes, wherein the probes are specific for the determination of the methylation status of at least 80% of the regions selected from Table 3, preferably all regions selected from Table 3.
   21. The device according to claim 20, wherein the device is a microarray.
   22. Use of the device of claim 20 or 21 for a method for
a) determining a score indicative of the diagnosis of a neurodegenerative disease or disorder of a subject, of the probability of a subject to develop a neurodegenerative disease or disorder and/or of the disease progression of a neurodegenerative disease or disorder; and/or
b) monitoring a neurodegenerative disease or disorder.
   23. Use of the device of claim 20 or 21 for a method according to claim 1 to 13.

Accordingly, the invention relates to a method for determining a score indicative of the diagnosis of a neurodegenerative disease or disorder of a subject, of the probability of a subject to develop a neurodegenerative disease or disorder and/or of the disease progression of a neurodegenerative disease or disorder, the method comprising the steps of: a) obtaining at least one methylation status of two or more genes selected from Table 1 and/or two or more regions selected from Table 2 from a sample of a subject; b) comparing the methylation status obtained in (a) to a reference pattern; and c) determining a score indicative of the diagnosis of a neurodegenerative disease or disorder of the subject, of the probability of the subject to develop a neurodegenerative disease or disorder and/or of the disease progression of a neurodegenerative disease or disorder based on the comparison obtained in (b).

The term “neurodegenerative disease or disorder”, as used herein, refers to a group of disease or disorders of the nervous system which are characterised by damage and/or death of neuronal subtypes. In some embodiments, the neurodegenerative disease or disorder described herein is a disease or disorder having impaired cognition as a symptom or a disease or disorder being characterized by impaired cognition. In some embodiments, the neurodegenerative disease or disorder described herein is a disease or disorder having dementia as a symptom or a disease or disorder being characterized by dementia. In some embodiments, the dementia described herein is caused by Alzheimer's disease, tauopathies, vascular dementia, Lewy Body disease, frontotemporal dementia, alcohol related dementia, down syndrome, HIV associated dementia, chronic traumatic encephalopathy (CTE) dementia and childhood dementia. In some embodiments, the neurodegenerative disease or disorder described herein is at least one disease or disorder selected from the group of dementia, Alzheimer's disease, Parkinson's disease, amyotrophic lateral sclerosis, multiple sclerosis, Huntington's disease, and prion disease.

The term “reference pattern”, as used herein, refers to a predetermined pattern that can be used for comparison and is preferably obtained from reference subjects. The reference pattern comprises at least one datapoint, such as a datapoint that can be used as a threshold. In some embodiments, the reference pattern is a (machine learning) model.

The term “score”, as used herein, refers to a value, a category, a diagnosis and/or a classification.

The term “sample”, as used herein, refers to any biological sample of a subject potentially comprising nucleic acid. In some embodiments, the sample is a sample selected from the group of bronchoalveolar lavage, bronchial wash, pharyngeal exudate, tracheal aspirate, blood, serum, plasma, bone, skin, soft tissue, intestinal tract specimen, genital tract specimen, breast milk, lymph, cerebrospinal fluid, pleural fluid, sputum, urine, a nasal secretion, tears, bile, ascites fluid, pus, synovial fluid, vitreous fluid, vaginal secretion, semen and urethral tissue. In some embodiments, the sample described herein is a sample selected from the group of blood sample, serum sample, plasma sample and urine sample. The sample may provide the methylation status of the genes or regions described herein in any determinable form. In some embodiments, the sample comprises genomic DNA and/or cell free DNA. In some embodiments, the methylation status of genes or regions is determined in the cell free DNA of the sample or in the cell free DNA and the genomic DNA. In some embodiments, the genomic DNA is isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA.

Wherein the sample DNA is not enclosed in a cell or membrane (e.g. circulating DNA from a blood sample) methods standard in the art for the isolation and/or purification of DNA may be employed. Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. The person skilled in the art may also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

The term “methylation” as used herein, refers to the covalent attachment of a methyl group at the C5-position of the nucleotide base cytosine within the CpG dinucleotides of gene regulatory region. The term “methylation state” or “methylation status” refers to the presence or absence of i) 5-methyl-cytosine (“5-mCyt”) or ii) 5-hydroxy-methyl-cytosine (5-hmC) at one or a plurality of CpG dinucleotides within a DNA sequence and N6-methyladenine (6-mA). As used herein, the terms “methylation status” and “methylation state” are used interchangeably. A methylation site is a sequence of contiguous linked nucleotides that is recognized and methylated by a sequence-specific methylase. A methylase is an enzyme that methylates (i.e., covalently attaches a methyl group) one or more nucleotides at a methylation site. Methylation status at one or more CpG methylation sites (each having two CpG dinucleotide sequences) or adenines within a DNA sequence include “unmethylated”, “fully-methylated” and “hemimethylated”. A variety of methylation analysis procedures are known in the art and may be used to practice the invention. The methylation status can be obtained by any method know in the art. These assays allow for determination of the methylation state of one or a plurality of CpG sites or adenines within a tissue sample. In addition, these methods may be used for absolute or relative quantification of methylated nucleic acids. Such methylation assays involve, among other techniques, two major steps. The first step is a methylation specific reaction or separation, such as (i) bisulfite treatment, (ii) methylation specific binding, (iii) methylation specific restriction enzymes and/or (iv) enzymatic conversion of methylated nucleic acids. The second major step involves (i) amplification and detection, or (ii) direct detection, by a variety of methods such as (a) PCR (sequence-specific amplification) such as Taqman®, (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

Additionally, restriction enzyme digestion of PCR products amplified from bisulfite-converted DNA may be used, e.g., the method described by Sadri and Hornsby (1996, Nucl. Acids Res. 24:5058-5059), or COBRA (Combined Bisulfite Restriction Analysis) (Xiong and Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Frommer et al, 1992, Proc. Nat. Acad. Sci. USA, 89, 1827-1831). PCR amplification of the bisulfite converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

In some embodiments, the methylation status of selected CpG sites is determined using MethyLight and Heavy Methyl Methods. The MethyLight and Heavy Methyl assays are a high-throughput quantitative methylation assay that utilizes fluorescence-based real-time PCR (Taq Man®) technology that requires no further manipulations after the PCR step (Eads, C. A. et al, 2000, Nucleic Acid Res. 28, e 32; Cottrell et al, 2007, J. Urology 177, 1753, U.S. Pat. No. 6,331,393 (Laird et al)). In some embodiments, the methylation status of selected CpG sites is determined using methylation-Specific PCR (MSP). MSP allows for assessing the methylation status of virtually any group of CpG sites within a CpG island, independent of the use of methylation-sensitive restriction enzymes (Herman et al., 1996, Proc. Nat. Acad. Sci. USA, 93, 9821-9826; U.S. Pat. Nos. 5,786,146, 6,017,704, 6,200,756, 6,265,171 (Herman and Baylin) U.S. Pat. Pub. No. 2010/0144836 (Van Engeland et al)).

Additionally, the enzymatic methyl-seq (EM-seq) technique can be used. Specifically, this technique selectively deaminates unmethylated cytosines to uracils to next generate and sequence the newly created libraries based on input DNA. Whole whole-genome sequencing or specific gene loci sequencing can be applied (Hoppers, Amanda et al. 2020, Journal of Biomolecular Techniques: JBT vol. 31, Suppl: S15; Williams, Louise, et al., 2019, “Enzymatic Methyl-seq: the next generation of methylome analysis.” NEB expressions).

The term “subject”, as used herein, refers to a mammal, such as a mouse, guinea pig, rat, dog or human. It is understood that the preferred subject is a human. In some embodiments, the subject is a human above the age of 40, preferentially in the age range of 40-75. The inventors found that the means and methods described herein are particularly effective in early detection, scoring and/or diagnosing of the neurodegenerative disease or disorder described herein. In some embodiments, the subject is a human having an age between 40 and 75, preferably between 40 and 65. In some embodiments, the subject is a human having no or no substantial cognitive symptoms or a human wherein the symptoms alone are insufficient to achieve a diagnose with high certainty (e.g. a certainty higher than 80%, 90% or 95%) for the respective neurodegenerative disease or disorder. In some embodiments, the subject described herein has an increased risk for developing a neurodegenerative disease or disorder. A subject having an increased risk for developing a neurodegenerative disease or disorder is for example a subject having an above average exposure to at least one risk factor or an above average number of risk factors selected from the group consisting of cardiovascular disease, cerebrovascular disease, smoking, prior head injury, genetics, diet, sleep deprivation, alcohol use, depression, poor fitness, high blood pressure and uncontrolled diabetes.

The term to “develop”, as used herein in the context of a neurodegenerative disease or disorder, refers to developing at least one symptom of a neurodegenerative disease or disorder. In some embodiments, developing a neurodegenerative disease or disorder described herein refers to developing enough symptoms to qualify for diagnosis.

In some embodiments, the invention relates to a method for diagnosing a subject with neurodegenerative disease or disorder, the method comprising the steps of: a) obtaining at least one methylation status of two or more genes selected from Table 1 and/or two or more regions selected from Table 2 from a sample of a subject; b) comparing the methylation status obtained in (a) to a reference pattern; and c) diagnosing a subject with neurodegenerative disease or disorder based on the comparison obtained in (b).

In some embodiments, the invention relates to a method for diagnosing a subject with neurodegenerative disease or disorder, the method comprising the steps of: a) obtaining at least one methylation status of two or more genes selected from Table 1 and/or two or more regions of Table 2 from a sample of a subject; b) comparing the methylation status obtained in (a) to a reference pattern; and c) diagnosing a subject with neurodegenerative disease or disorder based on the comparison obtained in (b).

The inventors found that epigenetic marks change in a specific way in neurodegenerative diseases or disorders. Neurodegeneration and/or cell death is/are reflected in the methylation status of two or more genes selected in Table 1 and/or in two or more regions of Table 2. The invention provides an approach that can be implemented at low cost and minimally invasive using a method comprising the use of a combination of diagnostic methylation statuses as described herein. Therefore, the invention provides epigenetic fingerprints as biomarkers of neurodegenerative diseases or disorders.

Accordingly, the invention is at least in part based on the finding that the combination of methylation statuses as described herein is particularly useful for the efficient, early and/or non-invasive detection of parameters relevant for diagnosing, for the prediction of the development and/or progression of neurodegenerative diseases or disorders.

In some embodiments, the invention relates to a method for distinguishing between diagnoses of a subject, the method comprising the steps of: a) obtaining at least one methylation status of two or more genes selected from Table 1 and/or two or more regions of Table 2 from a sample of a subject, wherein the subject has been diagnosed with at least two neurodegenerative disease or disorders and/or symptoms that are indicative of at least two neurodegenerative disease or disorders; b) comparing the methylation status obtained in (a) to a reference pattern; and c) distinguishing between diagnoses of the subject based on the comparison obtained in (b).

In some embodiments, the invention relates to a method for distinguishing between forms of dementia of a subject, the method comprising the steps of: a) obtaining at least one methylation status of two or more genes selected from Table 1 and/or two or more regions of Table 2 from a sample of a subject, wherein the subject has been diagnosed with at least two forms of dementia and/or symptoms that are indicative of at least forms of dementia; b) comparing the methylation status obtained in (a) to a reference pattern; and c) distinguishing between forms of dementia of the subject based on the comparison obtained in (b).

In some embodiments, the invention relates to a method for distinguishing in a subject Alzheimer's disease from other forms of dementia, the method comprising the steps of: a) obtaining at least one methylation status of two or more genes selected from Table 1 and/or two or more regions of Table 2 from a sample of a subject, wherein the subject has been diagnosed with at least two forms of dementia and/or symptoms that are indicative of at least two forms of dementia and wherein at least one form of dementia is Alzheimer's disease; b) comparing the methylation status obtained in (a) to a reference pattern; and c) distinguishing in the subject Alzheimer's disease from other forms of dementia based on the comparison obtained in (b).

In certain embodiments, the invention relates to the method of the invention, wherein the sample is a body fluid sample.

In certain embodiments, the invention relates to the method of the invention, wherein the sample is a sample selected from the group of blood sample, serum sample, plasma sample and urine sample.

In certain embodiments, the invention relates to the method of the invention, wherein the sample is a plasma sample.

The inventors found that plasma samples comprise sufficient information about the methylation status described herein to be indicative for the development and/or progression of a neurodegenerative disease or disorder. The obtainment of plasma samples is fast and simple and enables inexpensive and scalable early screening.

Accordingly, the invention is at least in part based on the finding that the method is particular efficient by using a body fluid sample such as a plasma sample as described herein.

In certain embodiments, the invention relates to the method of the invention wherein the sample is or was frozen.

The sample may for example have a known history of being frozen or may be freshly defrosted to apply the method of the invention. Analysing frozen samples enables stable storage over time. This storage enables for example retrospective diagnosis or analysis of disease progression over time (with two measuring time points). Analysis of past disease progression can help to estimate the future disease progression.

Accordingly, the invention is at least in part based on the finding that the method of the invention can be applied to a previously frozen sample.

In certain embodiments, the invention relates to the method of the invention, wherein the methylation status is obtained from cell free-DNA in the sample.

The terms “circulating DNA”, “cell free DNA”, “cfDNA”, “circulating cell free DNA” and “ccfDNA” are used herein interchangeably and refer to free DNA molecules of 25 nucleotides or longer that are not contained within any intact cell or membrane. In certain embodiments, the cfDNA described herein has a minimal length of at least 50 nucleotides, at least 75 nucleotides, at least 100 nucleotides or at least 125 nucleotides.

The presence of free circulating fragmented DNA in the bloodstream has been reported decades ago. However, cfDNA and/or methylation thereof was not expected to be sufficient for the early detection of diseases. For example, the use of liquid biopsies for the detection of circulating tumor DNA (ctDNA) is based on identifying somatic mutations, which accumulates in cancer cells and thus lacks sensitivity to detect early-stage cancer with a limited extent of recurrent mutations (Kustanovich, A., et al., 2019, Cancer Biology & Therapy 20, 1057-1067).

The inventors surprisingly found that cell free-DNA in samples (e.g. body fluid samples such as plasma samples) is useful for determining a methylation status in the context of neurodegenerative diseases or disorders including early detection thereof.

Accordingly, the advantage of determining cfDNA epigenetic markers (e.g. in plasma) is the use of i) a minimally invasive technique, ii) a signature integrating multiple modified molecules, iii) integrating the analysis of neurodegenerative disease or disorder-associated, iv) longitudinal analysis of disease progression in the same patient.

In certain embodiments, the invention relates to the method of the invention, wherein obtaining at least one methylation status comprises genome methylation profiling.

The term “genome methylation profiling”, as used herein, refers to a set of data representing the methylation states of at least 2, at least 3, at least 4, at least 5 or all loci within a molecule of DNA. The profile can indicate the methylation state of every base in an individual, can have information regarding a subset of the base pairs in a genome, or can have information regarding regional methylation density of each locus.

Accordingly, the invention is at least in part based on the finding that using genome methylation profiling enables the method described herein to be particularly sensitive/specific.

In certain embodiments, the invention relates to the method of the invention, wherein the at least one methylation status comprises the methylation status of at least 100 regions selected from Table 2, preferably at least 150 regions selected from Table 2, more preferably at least 200 regions selected from Table 2.

The inventors found that analysing a certain number of relevant regions results in a particular accurate result.

In certain embodiments, the invention relates to the method of the invention, wherein the at least one methylation status comprises the methylation status of between 200 and 600 regions selected from Table 2, preferably 250 and 500, more preferably between 250 and 300 regions selected from Table 2.

The inventors identified that certain ranges of number of regions provide a particular beneficial combination of accuracy and efficiency.

In certain embodiments, the invention relates to the method of the invention, wherein the at least one methylation status comprises the methylation status of at least 80% of the regions selected from Table 3, preferably 85% of the regions selected from Table 3, preferably 90% of the regions selected from Table 3, more preferably 95% of the regions selected from Table 3, more preferably all regions selected from Table 3.

The inventors identified a set of regions that enables the means and methods of the invention to be particularly accurate. Certain regions (e.g. 20%, 15%, 10% or 5%) of this set can be omitted or replaced by other regions (e.g. similar regions or close regions) without substantially affecting the performance of the set of regions as such.

In certain embodiments, the invention relates to the method of the invention, wherein the methylation status is determined in regions with a) an average nucleotide width of less than 5000, preferably less than 4000, more preferably less than 3000, more preferably less than 2000, more preferably less than 1000; and b) a median nucleotide width of less than 5000, preferably less than 4000, more preferably less than 3000, more preferably less than 2000, more preferably less than 1000.

In certain embodiments, the invention relates to the method of the invention, wherein the methylation status is determined in regions with a median nucleotide width of less than 5000, preferably less than 4000, more preferably less than 3000, more preferably less than 2000, more preferably less than 1000.

In certain embodiments, the invention relates to the method of the invention, wherein the methylation status is determined in regions with an average nucleotide width of less than 5000, preferably less than 4000, more preferably less than 3000, more preferably less than 2000, more preferably less than 1000.

The inventors found that using small regions improves the accuracy of the means and methods of the invention.

In certain embodiments, the invention relates to the method of the invention, wherein at least one, at least two or at least three further marker(s) is/are obtained in (a) compared in (b).

In some embodiments, the further markers comprise at least one clinical parameter and/or at least one pathological feature.

In certain embodiments, the invention relates to the method of the invention, wherein the at least one further marker is a marker selected from the group of subject background data, cognitive performance marker, autonomic nervous system biomarker.

The term “subject background data”, as used herein refers to characteristics of a subject that for technical or temporal reasons cannot be obtained by the methods that are used to determine the methylation status.

In some embodiments, the subject background data described herein is a subject background parameter selected from the group of age, gender, family history of neurodegenerative conditions.

The term “cognitive performance marker”, as used herein refers to a marker for mental functions including, for example, learning, problem solving, remote memory, recent memory, word comprehension, orientation, attention span, calculation, abstract thinking, and judgment. In some embodiments, the cognitive performance marker described herein is a memory performance marker. In some embodiments, the cognitive performance marker described herein is a maker selected from the group of the GPCOG, the Mini-Cog, the “Eight-item Informant Interview to Differentiate Aging and Dementia” and the “Short Informant Questionnaire on Cognitive Decline in the Elderly”. In some embodiments, the cognitive performance marker described herein comprises MMSE and/or MoCa scores.

The term “autonomic nervous system biomarker”, as used herein, refers to any nervous system-related data that can be obtained from a subject by a sensor or by a protein measurement, that does not require the subject to enter the data actively and consciously. In some embodiments, the autonomic nervous system biomarker described herein comprises an imaging marker such as positron-emission tomography (PET) scan or MRI. In some embodiments relating to the invention in the context of Parkinson's disease (e.g. the method of the invention for scoring, diagnosing or distinguishing Parkinson's disease), the autonomic nervous system biomarker described herein comprises movement-related data such as data indicative of gait or tremor. In some embodiments, the autonomic nervous system biomarker described herein is a marker selected from the group of positron-emission tomography (PET) scan, amyloid in CSF measurements and tau protein in CSF measurements.

The inventors found that the above described further markers can increase the resolution with which the disease is characterized.

Accordingly, the invention is at least in part based on the finding that the further markers described herein can increase the sensitivity and/or specificity of the methods described herein.

In certain embodiments, the invention relates to the method of the invention, wherein the reference pattern is obtained from the methylation status of two or more genes selected from Table 1 and/or at least two regions selected from Table 2 from samples of at least two reference subjects, wherein at least one of the reference subjects suffers from a neurodegenerative disease or disorder.

The inventors found, that using data of subjects suffering from a neurodegenerative disease or disorder can be used as a reference. Preferably the reference subjects consist at least in part of subjects having the same neurodegenerative disease or disorder as the subjects or sample of subjects for which a determination, a prediction or a monitoring is done according to the methods described herein.

Accordingly, the invention is at least in part based on the finding that data from diseased subject is particularly useful for the reference pattern in the methods described herein.

In certain embodiments, the invention relates to the method of the invention, wherein the reference pattern is obtained from the methylation status of two or more genes selected from Table 1 and/or two or more regions selected from Table 2 from samples of at least two reference subjects, wherein at least one of the reference subjects suffers from a neurodegenerative disease or disorder and wherein at least one of the reference subjects does not suffer from a neurodegenerative disease or disorder, preferably wherein the at least one of the reference subjects does not suffer from a neurodegenerative disease or disorder is a healthy subject.

In certain embodiments, the invention relates to the method of the invention, wherein the samples of at least two reference subjects comprise at least one brain tissue sample.

The term “brain tissue sample”, as used herein, refers to a sample of the CNS such as a cortical brain tissue sample.

The inventors found that brain tissue samples provide information regarding the disease and that this information can be used to identify disease-related methylation pattern outside the brain tissue. Therefore, the invasive procedure of obtaining a brain tissue sample can be limited to the reference subjects. The disease-related information comprised in the brain tissue sample(s) can reduce the number of reference subjects needed to achieve a certain sensitivity/specificity.

Accordingly, the invention is at least in part based on the finding that brain tissue sample comprise information that improves the sensitivity/specificity.

In certain embodiments, the invention relates to the method of the invention, wherein obtaining the reference pattern from the methylation status of two or more genes selected from Table 1 and/or two or more regions selected from Table 2 from samples of at least two reference subjects comprises a machine learning technique.

The term “machine-learning technique”, as used herein, refers to a computer-implemented technique that enables automatic learning and/or improvement from an experience (e.g., training data and/or obtained data) without the necessity of explicit programming of the lesson learned and/or improved. In some embodiments, the machine leaning technique described herein is an artificial intelligence technique. In some embodiments, the machine learning comprises at least one technique selected from the group of Logistic regression, CART, Bagging, Random Forest, Gradient Boosting, Linear Discriminant Analysis, Gaussian Process Classifier, Gaussian NB, Linear, Lasso, Ridge, ElasticNet, partial least squares, KNN, DecisionTree, SVR, Support Vector Machine, AdaBoost, GradientBoost, neural net, ExtraTrees, Fuzzy neural network, Linear Regression, Decision Tree, Naive Bayes and K-Means.

The inventors found that machine-learning techniques provide an efficient and/or unbiased way to identify fingerprints in the context of a neurodegenerative disease or disorder. These fingerprints can be general for one or more neurodegenerative diseases or disorders.

In certain embodiments, the invention relates to a method for monitoring a neurodegenerative disease or disorder, the method comprising the steps of:
i) determining a first score indicative of the probability of a subject to develop a neurodegenerative disease or disorder and/or of the disease progression of a neurodegenerative disease or disorder according to the method of the invention at a first timepoint; ii) determining a second score indicative of the probability of a subject to develop a neurodegenerative disease or disorder and/or of the disease progression of a neurodegenerative disease or disorder according to the method of the invention at a second timepoint; iii) comparing the first score of step (i) with the second score of step (ii); and iv) monitoring the disease progression of the neurodegenerative disease or disorder in the subject based on the comparison of step (iii).

Accordingly, the invention is at least in part based on the finding that the method described herein can be used to provide temporal information about the neurodegenerative disease or disorder.

In certain embodiments, the invention relates to a library comprising at least one score indicative of the probability of a subject to develop a neurodegenerative disease or disorder and/or of the disease progression of a neurodegenerative disease or disorder determined according to the invention.

In some embodiments, the library described herein is a machine learning model trained on at least one score indicative of the probability of a subject to develop a neurodegenerative disease or disorder and/or of the disease progression of a neurodegenerative disease or disorder determined according to the invention.

The present invention may be a system, a method, and/or a computer program product.

The computer program product may include a computer-readable storage medium (or media) having the computer-readable program instructions thereon for causing a processor to carry out embodiments of the invention such as the computer-implemented method for classification according to the invention and/or the computer-implemented method for obtainment according to the invention.

In certain embodiments, the invention relates to a storage device comprising computer-readable program instructions to execute the method according to the invention, preferably additionally comprising the library of the invention.

Computer-readable program instructions described herein can be downloaded to respective computing/processing devices from a computer-readable storage medium or to an external computer or external storage device via a network.

Computer-readable program instructions for carrying out operations of the present invention may be assembler instructions, instruction-set-architecture (ISA) instructions, machine instructions, machine-dependent instructions, microcode, firmware instructions, state-setting data, or either source code or object code written in any combination of one or more programming languages, including an object-oriented programming language such as Smalltalk, C++ or the like, and conventional procedural programming languages, such as the “C” programming language or similar programming languages.

The computer-readable program instructions may execute entirely on the user's computer, partly on the user's computer, as a stand-alone software package, partly on the user's computer and partly on a remote computer or entirely on the remote computer or server.

The term “storage device”, as used herein, refers to any tangible device that can retain and store instructions for use by an instruction execution device.

In some embodiments, the storage device described herein is at least one selected from the group of electronic storage device, magnetic storage device, optical storage device, electromagnetic storage device, semiconductor storage device, any suitable combination thereof.

A non-exhaustive list of more specific examples of the storage device includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A storage device, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In certain embodiments, the invention relates to a server comprising the storage device of the invention, at least one processing device, and a network connection for receiving data indicative of at least one methylation status.

The term “data indicative of at least one methylation status”, as used herein, refers to any raw or processed data that describes the methylation status and/or properties of the methylation status.

The term “network connection”, as used herein, refers to a communication channel of a data network. A communication channel can allow at least two computing systems to communicate data to one another. In some embodiments, the data network is selected from the group of the internet, a local area network, a wide area network, and a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer-readable program instructions for storage in a computer-readable storage medium within the respective computing/processing device.

The server described herein, can receive the data indicative of at least one methylation status, process it according to the method of the invention, and provide a result. Sending the data indicative of at least one methylation status to a server reduces the requirements for processing power in the device that acquires the data indicative of at least one methylation status and enables the efficient processing of large datasets. In embodiments, wherein the invention relates to a server, the data indicative of at least one methylation status can be obtained acquired by any device that has a network connection.

The server may be connected to the device for the acquirement of the data indicative of at least one methylation status through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform embodiments of the present invention.

Accordingly, the server described herein, enables the efficient application of the methods of the invention.

In certain embodiments, the invention relates to the method of the invention, the library of the invention, the storage device of the invention, the server of the invention, wherein the neurodegenerative disease or disorder is Alzheimer's disease and/or Parkinson's disease.

The term “Alzheimer's disease” or “AD”, as used herein, refers to mental deterioration associated with a specific degenerative brain disease that is characterized by senile plaques, neuritic tangles and progressive neuronal loss which manifests clinically in progressive memory deficits, confusion, behavioral problems, inability to care for oneself and/or gradual physical deterioration.

In some embodiments, subjects suffering Alzheimer's disease are identified using the NINCDS-ADRDA (National Institute of Neurological and Communicative Disorders and the Alzheimer's Disease and Related Disorders Association) criteria:

1) Clinical Dementia Rating (CDR)=1; Mini Mental State Examination (MMSE) between 16 and 24 points and Medial temporal atrophy (determined by Magnetic Resonance Imaging, MRI) >3 points in Scheltens scale. In some embodiments, the term Alzheimer's disease includes all the stages of the disease, including the following stages defined by NINCDS-ADRDA Alzheimer's Criteria for diagnosis in 1984.
2) Definite Alzheimer's disease: The patient meets the criteria for probable Alzheimer's disease and has histopathologic evidence of AD via autopsy or biopsy.

Probable or prodromal Alzheimer's disease: Dementia has been established by clinical and neuropsychological examination. Cognitive impairments also have to be progressive and be present in two or more areas of cognition. The onset of the deficits has been between the ages of 40 and 90 years and finally there must be an absence of other diseases capable of producing a dementia syndrome.

3) Possible or non-prodromal Alzheimer's disease: There is a dementia syndrome with an atypical onset, presentation; and without a known etiology; but no co-morbid diseases capable of producing dementia are believed to be in the origin of it. In some embodiments, the term Alzheimer's disease refers one stage of Alzheimer's disease. In some embodiments, the term Alzheimer's disease refers to two stages of Alzheimer's disease. In some embodiments, the term “Alzheimer's disease” refers to symptoms of Alzheimer's disease, which include without limitation, loss of memory, confusion, difficulty thinking, changes in language, changes in behavior, and/or changes in personality.

The term “Parkinson's disease”, as used herein, refers to a neurological syndrome characterized by a dopamine deficiency, resulting from degenerative, vascular, or inflammatory changes in the basal ganglia of the substantia nigra. Symptoms of Parkinson's disease include, without limitation, the following: rest tremor, cogwheel rigidity, bradykinesia, postural reflex impairment, good response to 1-dopa treatment, the absence of prominent oculomotor palsy, cerebellar or pyramidal signs, amyotrophy, dyspraxia, and/or dysphasia. In a specific embodiment, the present invention is utilized for the treatment of a dopaminergic dysfunction-related syndrome. In some embodiments, Parkinson's disease includes any stage of Parkinson's disease. In some embodiments, the term Parkinson's disease includes the early stage of Parkinson's disease, which refers broadly to the first stages in Parkinson's disease, wherein a person suffering from the disease exhibits mild symptoms that are not disabling, such as an episodic tremor of a single limb (e.g., the hand), and which affect only one side of the body.

In some embodiments, the term Parkinson's disease includes the advanced stage of Parkinson's disease, which refers to a more progressive stage in Parkinson's disease, wherein a person suffering from the disease exhibits symptoms which are typically severe and which may lead to some disability (e.g., tremors encompassing both sides of the body, balance problems, etc.). Symptoms associated with advanced-stage Parkinson's disease may vary significantly in individuals and may take several years to manifest after the initial appearance of the disease.

In some embodiments, the term “Parkinson's disease” refers to symptoms of Parkinson's disease, which include without limitation, tremors (e.g., tremor which is most pronounced during rest), shaking (e.g. trembling of hands, arms, legs, jaw and face), muscular rigidity, lack of postural reflexes, slowing of the voluntary movements, retropulsion, mask-like facial expression, stooped posture, poor balance, poor coordination, bradykinesia, postural instability, and/or gait abnormalities.

In certain embodiments, the invention relates to the method of the invention, the library of the invention, the storage device of the invention, the server of the invention, wherein the neurodegenerative disease or disorder is Alzheimer's disease.

In certain embodiments, the invention relates to a device comprising methylation specific oligonucleotide probes, wherein the probes are specific for the determination of the methylation status of at least 80% of the regions selected from Table 3, preferably all regions selected from Table 3.

In certain embodiments, the invention relates to the device according to the invention, wherein the device is a microarray.

In certain embodiments, the invention relates to the use of the device of the invention for a method for a) determining a score indicative of the diagnosis of a neurodegenerative disease or disorder of a subject, of the probability of a subject to develop a neurodegenerative disease or disorder and/or of the disease progression of a neurodegenerative disease or disorder.

In certain embodiments, the invention relates to the use of the device of the invention for a method for monitoring a neurodegenerative disease or disorder.

In certain embodiments, the invention relates to the use of the device of the invention for a method for a) determining a score indicative of the diagnosis of a neurodegenerative disease or disorder of a subject, of the probability of a subject to develop a neurodegenerative disease or disorder and/or of the disease progression of a neurodegenerative disease or disorder; and b) monitoring a neurodegenerative disease or disorder.

In certain embodiments, the invention relates to the use of the device of the invention for a method according to the invention.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

The terms “about” or “approximately”, as used herein, refer to “within 20%”, more preferably “within 10%”, and even more preferably “within 5%”, of a given value or range.

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention further relates to, the following items:

1. A method for determining a score indicative of the diagnosis of a neurodegenerative disease or disorder of a subject, of the probability of a subject to develop a neurodegenerative disease or disorder and/or of the disease progression of a neurodegenerative disease or disorder, the method comprising the steps of:
a) obtaining at least one methylation status of two or more genes selected from Table 1 and/or two or more genes selected from Table 2 from a sample of a subject;
b) comparing the methylation status obtained in (a) to a reference pattern; and c) determining a score indicative of the diagnosis of a neurodegenerative disease or disorder of the subject, of the probability of the subject to develop a neurodegenerative disease or disorder and/or of the disease progression of a neurodegenerative disease or disorder based on the comparison obtained in (b).
   2. The method of item 1, wherein the sample is a plasma sample.
   3. The method of item 1 or 2, wherein the methylation status is/are obtained from cell free-DNA in the sample.
   4. The method of any one of items 1 to 3, wherein obtaining at least one methylation status comprises genome methylation profiling.
   5. The method of any one of items 1 to 4, wherein at least one further marker is obtained in (a) compared in (b).
   6. The method of item 5, wherein the at least one further marker is a marker selected from the group of subject background data, cognitive performance marker, autonomic nervous system biomarker.
   7. The method of any one of items 1 to 6, wherein the reference pattern is obtained from the methylation status of two or more genes selected from Table 1 and/or wo or more regions selected from Table 2 from samples of at least two reference subjects, wherein at least one of the reference subjects suffers from a neurodegenerative disease or disorder.
   8. The method of item 7, wherein the samples of at least two reference subjects comprise at least one brain tissue sample.
   9. The method of item 7 or 8, wherein obtaining the reference pattern from the methylation status of two or more genes selected from Table 1 and/or two or more regions selected from Table 2 from samples of at least two reference subjects comprises a machine learning technique.
   10. A method for monitoring a neurodegenerative disease or disorder, the method comprising the steps of:
i) determining a first score indicative of the probability of a subject to develop a neurodegenerative disease or disorder and/or of the disease progression of a neurodegenerative disease or disorder according to the method of any one of items 1 to 9 at a first timepoint;
ii) determining a second score indicative of the probability of a subject to develop a neurodegenerative disease or disorder and/or of the disease progression of a neurodegenerative disease or disorder according to the method of any one of items 1 to 9 at a second timepoint;
iii) comparing the first score of step (i) with the second score of step (ii); and
iv) monitoring the disease progression of the neurodegenerative disease or disorder in the subject based on the comparison of step (iii).
   11. A library comprising at least one score indicative of the probability of a subject to develop a neurodegenerative disease or disorder and/or of the disease progression of a neurodegenerative disease or disorder determined according to any one of the items 1 to 9.
   12. A storage device comprising computer-readable program instructions to execute the method according to any one of the items 1 to 9, preferably additionally comprising the library of item 11.
   13. A server comprising the storage device of item 12, at least one processing device, and a network connection for receiving data indicative of at least one methylation status.
   14. The method of any one of items 1 to 10, the library of item 11, the storage device of item 12, the server of item 13, wherein the neurodegenerative disease or disorder is Alzheimer's disease and/or Parkinson's disease.
   15. The method of item 14, the library of item 14, the storage device of item 14, the server of item 14, wherein the neurodegenerative disease or disorder is Alzheimer's disease.

### BRIEF DESCRIPTION OF FIGURES

FIG. 1: cross-validated AUC distribution (median=0.99)

FIG. 2: number of features included (median=274)

FIG. 3: AUC validation set distribution (median=0.83)

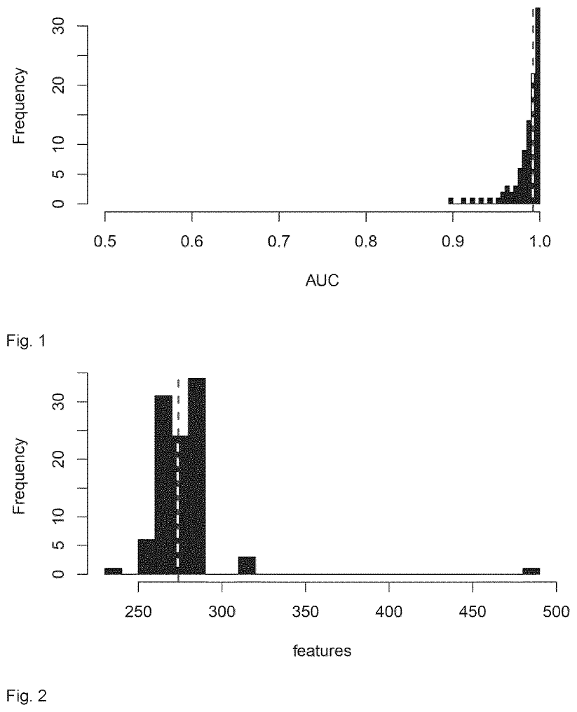

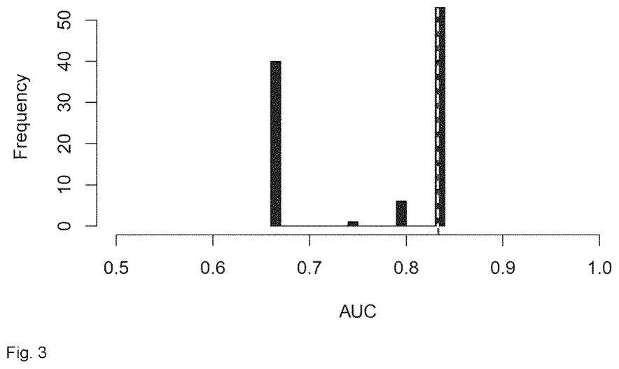

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1

### Project Objectives and Design

### Hypothesis and Primary Objective

Our working hypothesis is that an ongoing neurodegenerative process leads to detectable brain-derived cfDNA circulating in the plasma carrying an epigenetic signature precisely reflecting the origin as well as the pathogenic mechanisms causing and resulting from AD.

The primary objective of this observational pilot study is to reach a proof of concept of an in vitro diagnosis for AD based on the analysis of cfDNA.

### Primary Endpoint

The primary endpoint is the difference in total amount of cfDNA in plasma and its epigenetic signature between patients with AD and healthy subjects.

The total amount of cfDNA present in plasma as well as its epigenetic signature are expected to significantly differ between age- and gender-matched healthy controls and patients with clinically diagnosed AD in a phase of the disease displaying a significant rate of neuronal and synaptic loss. This will allow obtaining an epigenetic signature of AD that can be used for in vitro diagnostics (IVD) diagnosis.

### Project Design

This observational pilot study is based on a single collection of blood samples from two cohorts of participants, one with an AD diagnosis and the other of age- and gender matched healthy subjects. The blood samples are utilized for the purification of cfDNA from plasma and the analysis of epigenetic status at a whole genome level.

The research project will recruit up to 50 participants: 30 AD patients and 20 age- and gender-matched controls.

### Project Population and Study Procedures

### Project Population, Inclusion and Exclusion Criteria

### Project Populations:

Patients with Clinically Diagnosed AD

### Inclusion Criteria:

Clinical diagnosis of AD, Age: 40 years or older, probable AD dementia and with evidence of an AD pathophysiological process based on international guidelines (McKhann, G. M. et at., 2011, Alzheimers Dement 7, 263-269)—or Mild Cognitive Impairment (MCI) due to AD based on international guidelines (Albert, M. S. et al., 2011, Alzheimers Dement 7, 270-279).

### Healthy Age- and Gender-Matched Subjects

### Inclusion Criteria:

Healthy subject, age- and gender-matched with the AD patient population, no known disease, preserved cognitive capacity

### Exclusion Criteria for Both Populations:

Other neurodegenerative disorders or cause of cognitive decline

### Recruitment, Screening and Informed Consent Procedure

The participants to the study will be enrolled at the “Unita disturbi cognitivi e logopedia Neurocentro”, Istituto di Neuroscienze Cliniche della Svizzera Italiana, Ente Ospedaliero Cantonale. Healthy volunteers will be recruited among patient relatives or staff members and their families.

Participants fulfilling inclusion criteria for the two cohorts (AD, healthy) will be contacted and asked to participate to the research project.

### Study Procedures

One visit for blood draws at the “Unita disturbi cognitivi e logopedia Neurocentro”, Istituto di Neuroscienze Cliniche della Svizzera Italiana, Ente Ospedaliero Cantonale.

Blood samples will be collected in 5×10 ml_(50 ml) PAXgene Blood ccfDNA Tubes, Qiagen. cfDNA will next be purified from plasma using QlAamp Circulating Nucleic Acid Kit and its quantity determined by fluorescence-based commercial kits. cfDNA will be sequenced according to standard protocols of methylated DNA (e.g. chemistry-basted methylation sequencing and enzyme-based methylation sequencing) (Illumina).

Healthy subjects will be also undergone a diagnostic test by means of a questionnaire or an interview with the neurologist to exclude the presence of any sign or symptom of cognitive decline.

### Protocol #1

1. Draw 10 mL-20 mL blood from subject
2. Separate Plasma by traditional centrifugation steps
3. Purify cfDNA (10 ng-1 ug) from plasma sample using molecular biology techniques such as column-based purification of nucleic acids.
4. cfDNA quality assessment using dye-based by fluorescence assays
5. Bisulfite Conversion of cfDNA (e.g. using e Zymo EZ DNA Methylation Lightning Kit)
6. Purification of the bisulfite-treated DNA on a spin column
7. Preparation of sequencing library (e.g. using the EpiGnome™ Kit by Epicentre)
8. Sequencing of the library (e.g. using HiSeq 2500 System)
9. Creation of sequencing files (e.g. FASTQ files)
10. Annotation of sequenced methylated DNA on human reference genome (e.g. bisulfite-converted UCSC HG19 reference genome)
11. Statistical analysis to determine differential methylation calculations.
12. Comparison by computational methods or machine learning algorithms (e.g. Random Forest) of methylation signature of AD and healthy subjects
13. Identification of methylation signature: a list of genes whose methylated state is altered in AD subjects.

### Protocol #2

1. Draw 20 mL blood from subject
2. Separate Plasma by traditional centrifugation steps
3. Purify cfDNA (10 ng-1 ug) from plasma sample using molecular biology techniques such as column-based purification of nucleic acids.
4. cfDNA quality assessment using dye-based by fluorescence assays
5. Bisulfite Conversion of cfDNA (e.g. using e Zymo EZ DNA Methylation Lightning Kit)
6. Purification of the bisulfite-treated DNA on a spin column
7. Preparation of sequencing library (e.g. using the EpiGnome™ Kit by Epicentre)
8. Sequencing of the library (e.g. using HiSeq 2500 System)
9. Generation of sequencing files (e.g. FASTQ files)
10. Read mapping on the human reference genome (e.g. bisulfite-converted UCSC GRCh38 reference genome)
11. Identification of methylation status at base resolution and quantification of the methylation status for annotated genomic regions (e.g. promoter region)
12. Statistical analysis to determine differential methylation levels between cases and controls.
13. Development of a class prediction model using machine learning algorithms (e.g. Random Forest) to discriminate AD and healthy subjects. This will include the selection of an optimal set of feature in a cross-validation setting followed by evaluation of the performance.
14. The selected feature will constitute the methylation signature: a list of genomic regions whose methylated state is altered in AD subjects.

### Protocol #3

1. Draw 20 mL blood from subject
2. Separate Plasma by traditional centrifugation steps
3. Purify cfDNA (10 ng-1 ug) from plasma sample using molecular biology techniques such as column-based purification of nucleic acids.
4. cfDNA quality assessment using dye-based by fluorescence assays
5. Bisulfite Conversion of cfDNA (e.g. using e Zymo EZ DNA Methylation Lightning Kit)
6. Purification of the bisulfite-treated DNA on a spin column
7. Preparation of sequencing library (e.g. using the EpiGnome™ Kit by Epicentre)
8. Sequencing of the library (e.g. using HiSeq 2500 System) to determine methylation status of genes in Table 1.
9. Generation of sequencing files (e.g. FASTQ files)
10. Read mapping on the human reference genome (e.g. bisulfite-converted UCSC GRCh38 reference genome)
11. Identification of methylation status at base resolution and quantification of the methylation status for annotated genomic regions (e.g. promoter region)
12. Statistical analysis to determine differential methylation levels between cases and controls.
13. Development of a class prediction model using machine learning algorithms (e.g. Random Forest) to discriminate AD and healthy subjects. This will include the selection of an optimal set of features in a cross-validation setting followed by evaluation of the performance. Both methylation signals and clinical parameters and pathological features will be considered as candidate features.
14. The selected feature will constitute the methylation signature: a list of genomic regions whose methylated state is altered in AD subjects.

### Analysis of the Markers of Table 1:

Based on the data from the samples of the subjects of the above-mentioned study, biomarker combination candidates will be identified from Table 1.

TABLE 1

| | | | | | | |
|---|---|---|---|---|---|---|
| SNCA | SMYD4 | BTBD11 | CRTC3 | FLJ32224 | LGMN | PALM |
| AANAT | SNORD68 | C10orf105 | CSRP3 | FLOT1 | LHFPL2 | PARVG |
| ABCA7 | SPG7 | C10orf11 | CTSZ | FMNL1 | LOC338799 | PAX6 |
| AF070569 | TLCD2 | C10orf54 | CUBN | FSTL3 | LOC339524 | PCBD1 |
| AGPAT6 | UBE2O | C10orf79 | CUX2 | GAS2L1 | LOC389333 | PCDH9 |
| AK291164 | WDR81 | MIR609 | CX3CL1 | GBP6 | LOC400931 | PDE4B |
| AK311383 | ABCC1 | C10orf99 | CXCL10 | GCNT2 | LOXL2 | PDGFRB |
| ANK1 | ACAP1 | C12orf61 | ART3 | GDF3 | LPIN1 | PDLIM2 |
| ANKRD11 | ACLY | C14orf109 | CYTH1 | GLRX | LPPR2 | PFN3 |
| AP5Z1 | ADK | C17orf64 | DDHD2 | GNG7 | LST1 | PHC2 |
| AX747521 | ADORA3 | C1orf150 | DDR1 | GRASP | MAD1L1 | PHF2 |
| BC035889 | AFF3 | C4orf19 | DEF6 | GSTA3 | MAMSTR | PITPNM2 |
| BIN1 | AGAP2 | C6orf123 | DENND1C | GSTA4 | MAP1LC3A | PLAT |
| CNN2 | AHDC1 | C9orf25 | DGKI | GTF2H4 | MAP4K1 | PLCB2 |
| CPNE7 | AMZ1 | DNAI1 | DHX16 | VARS2 | MBP | PLCB4 |
| CYGB | ANK1 | CACNA1C | DKFZp-434J0226 | GUCY2D | MCF2L | PLCE1 |
| DIP2A | MIR486 | CALCR | DLEU2 | GYPC | MED27 | PLCH1 |
| DQ655986 | ANKRD33B | CAMTA1 | DLEU7 | HLA-DPB1 | MEST | PLD4 |
| FOXK1 | ANO1 | CCDC42B | DOK3 | HLA-DRB1 | MGC29506 | PLEKHA6 |
| GPX4 | APTX | CCL3 | ECEL1P2 | HMHA1 | MIR138-2 | PPT2 |
| HOTAIRM1 | ARHGAP27 | CCM2 | EIF2AK4 | HOXA2 | MKNK1 | PRRT1 |
| HOXA1 | ARHGEF17 | CD53 | EME1 | HOXA4 | MLST8 | PRKAG2 |
| HOXA10 | ASB2 | CD69 | LRRC59 | HOXA5 | MMP11 | PRKCA |
| HOXA5 | ASPSCR1 | CD79B | EMID1 | HOXA6 | MPL | PRKCD |
| HOXA7 | ATG16L2 | CDH23 | EPB41L1 | HOXB3 | MPO | PRSS36 |
| HOXA9 | ATP2A3 | CDH9 | EPS8 | HOXB7 | MTA3 | PRSSL1 |
| ITPRIPL2 | ATP6V1E2 | CEBPE | ERC2 | HOXB9 | MTMR11 | PTK6 |
| JA429246 | ATP8B2 | CES2 | ESPNL | HSPC159 | MYH10 | RAB1A |
| LOC100133311 | AUTS2 | FAM96B | ESR1 | ICMT | MYO1F | RAB23 |
| MIR22 | AZU1 | CHI3L2 | ETV3L | ICT1 | MYO1G | RASA3 |
| MIR22HG | B3GNT7 | CHRM3 | ETV5 | IPCEF1 | NCRNA00115 | RAVER1 |
| NKX6-3 | BAIAP2 | CLDN15 | FAH | IRF7 | LOC643837 | ICAM3 |
| PCNT | BBS1 | CLYBL | FAM102B | MUPCDH | NDUFS6 | RBM33 |
| POLR2E | BCAN | CMYA5 | FAM163A | IRS2 | NDUFS8 | RGS3 |
| PRCD | BCL2 | CNNM2 | FAM96B | KCNE1 | NEGR1 | RHBDF2 |
| PRPF8 | BGLAP | CNTN1 | CES2 | KCTD12 | NR5A1 | RHOB |
| RADIL | BHLHA9 | CNTNAP1 | FBXL14 | KDM2A | NRXN2 | RIN3 |
| RHBDF2 | BLCAP | COL23A1 | FBXO21 | KDM2B | NTRK3 | RLTPR |
| RPL13 | BMP4 | COQ7 | FGFR2 | KIF21B | OAT | RNF112 |
| SBNO2 | BPI | CPEB3 | FGGY | KLF1 | OBSCN | RNF34 |
| SERPINF1 | BRIX1 | CPEB4 | FLJ14107 | KLF13 | ORAOV1 | RP9P |
| SERPINF2 | RAD1 | SIRT6 | TNNT3 | SLCO5A1 | UTP23 | STIM1 |
| RUNX2 | TBCD | SKAP1 | TNS3 | SLMAP | VARS2 | STK10 |
| RXRB | TEX14 | SLC15a4 | TOP1MT | SMOC1 | GTF2H4 | STK32C |
| SATB1 | TGFBRAP1 | SLC1A5 | TOX2 | SMURF1 | VPS13D | STX5 |
| SBK1 | THBS2 | SLC22A23 | TP73 | SNX5 | WDFY1 | NXF1 |
| SCN2B | THRA | SLC22A8 | TRAPPC3 | SNORD17 | WDR26 | TACC3 |
| SEC14L1 | TM4SF19 | SLC25A37 | TRIM59 | SORBS2 | ZBTB7A | TADA2B |
| SEMA4B | TMEM51 | SLC2A1 | TRIO | SPG7 | ZC3H18 | TAF4 |
| SERP2 | TMPRSS4 | SLC38A7 | TRPV4 | SPNS3 | ZDHHC12 | TAP1 |
| SEZ6 | TMX4 | SLC44A2 | TSPAN14 | SSTR3 | ZFPM1 | ZNF385A |
| SH3RF3 | TNKS1BP1 | SLC7A5 | TTC22 | ST5 | ZMAT4 | CREB5 |
| ZNF512 | ZNF710 | TBC1D14 | RTN1 | P2RX1 | LAG3 | BIN3 |

TABLE 2
(all positions of the regions/features are described herein in reference to the numbering of
the human reference genome GRCh38 with the GenBank assembly accession code GCA_000001405.15)

| Chr | Start | End | Chr | Start | End | Chr | Start | End |
|---|---|---|---|---|---|---|---|---|
| chr6 | 37648161 | 37650402 | chr17 | 47852735 | 47854752 | chr19 | 8731542 | 8732011 |
| chr11 | 117198968 | 117199615 | chr6 | 28730638 | 28733263 | chr1 | 30883835 | 30884323 |
| chr3 | 158329620 | 158330315 | chr5 | 168216716 | 168217748 | chr16 | 22624583 | 22625315 |
| chr1 | 10808891 | 10809856 | chr2 | 218899197 | 218900627 | chr14 | 76760377 | 76760869 |
| chr17 | 48603352 | 48605650 | chr11 | 69928038 | 69928606 | chr7 | 50653139 | 50653539 |
| chr6 | 167794851 | 167795750 | chr17 | 51834927 | 51837514 | chr2 | 95046463 | 95047550 |
| chr21 | 45256014 | 45257568 | chr22 | 44927456 | 44927902 | chr5 | 154493280 | 154493668 |
| chr9 | 62844696 | 62845119 | chr1 | 1896978 | 1897947 | chr5 | 7394195 | 7394897 |
| chr2 | 3594718 | 3595278 | chr9 | 135098429 | 135099039 | chr10 | 23062802 | 23063612 |
| chr7 | 39130864 | 39131581 | chr11 | 131977303 | 131980287 | chr21 | 44593720 | 44594738 |
| chr9 | 132543885 | 132544442 | chr11 | 3227905 | 3228626 | chr11 | 33575838 | 33577156 |
| chr1 | 178486665 | 178487359 | chr4 | 6345238 | 6345684 | chr2 | 129252498 | 129254361 |
| chr17 | 47844295 | 47849105 | chr22 | 49369093 | 49369593 | chr16 | 1089678 | 1090154 |
| chr5 | 77846558 | 77847470 | chr2 | 23629078 | 23629622 | chr6 | 39793000 | 39793566 |
| chr14 | 94864350 | 94864896 | chr8 | 37841284 | 37842550 | chr1 | 24730549 | 24731556 |
| chr2 | 9739008 | 9739863 | chr8 | 949599 | 950191 | chr12 | 104715796 | 104716245 |
| chr9 | 136311045 | 136312238 | chr2 | 222268222 | 222269081 | chr21 | 40189094 | 40194563 |
| chr4 | 184097238 | 184097681 | chr2 | 103308479 | 103311145 | chr7 | 140641622 | 140642516 |
| chr6 | 127514451 | 127514915 | chr17 | 79516319 | 79516850 | chr10 | 122645741 | 122647302 |
| chr1 | 2968467 | 2968824 | chr7 | 154768930 | 154769482 | chr3 | 13138165 | 13138374 |
| chr6 | 151808909 | 151809540 | chr6 | 167706453 | 167706838 | chr20 | 44190556 | 44191032 |
| chr7 | 72447027 | 72447547 | chr10 | 33298798 | 33300575 | chr22 | 18987226 | 18988028 |
| chr15 | 60008144 | 60010990 | chr11 | 2099883 | 2100474 | chr7 | 101902847 | 101903731 |
| chr3 | 14216389 | 14217120 | chr13 | 57581748 | 57582325 | chr2 | 236792878 | 236793938 |
| chr6 | 8277473 | 8279189 | chr12 | 128381760 | 128383007 | chr21 | 41063966 | 41065055 |
| chr14 | 101501279 | 101501779 | chr5 | 173230171 | 173230899 | chr16 | 67155977 | 67156619 |
| chr22 | 31104411 | 31105340 | chr8 | 46108987 | 46109582 | chr1 | 17403649 | 17404473 |
| chr22 | 19761657 | 19765088 | chr2 | 119169494 | 119170676 | chr17 | 73720154 | 73720758 |
| chr11 | 68649691 | 68650284 | chr1 | 2826099 | 2826685 | chr21 | 10729268 | 10729673 |
| chr8 | 64798980 | 64799609 | chr5 | 1638083 | 1638693 | chr17 | 55939122 | 55940621 |
| chr15 | 29871222 | 29871742 | chr5 | 504951 | 506359 | chr1 | 19661016 | 19661600 |
| chr10 | 14031952 | 14032795 | chr5 | 166962272 | 166963051 | chr19 | 55901780 | 55902638 |
| chr5 | 42943586 | 42944530 | chr16 | 2027797 | 2029618 | chr2 | 240996900 | 240997166 |
| chr11 | 133951074 | 133951993 | chr16 | 30011935 | 30012409 | chr17 | 39828670 | 39829055 |
| chr2 | 241774375 | 241775375 | chr2 | 239822158 | 239822755 | chr6 | 3745424 | 3746570 |
| chr22 | 46390475 | 46391190 | chr8 | 11392675 | 11393310 | chr1 | 205466911 | 205467689 |
| chr6 | 27743807 | 27744418 | chr5 | 882295 | 882750 | chr4 | 1227423 | 1228153 |
| chr11 | 128687986 | 128691501 | chr11 | 118724860 | 118727249 | chr7 | 132750808 | 132751479 |
| chr2 | 95027126 | 95027797 | chr18 | 10926442 | 10927443 | chr10 | 133124318 | 133125314 |
| chr12 | 66804099 | 66804948 | chr2 | 10922076 | 10922629 | chr15 | 70469321 | 70470182 |
| chr10 | 30058203 | 30058722 | chr15 | 91069761 | 91071163 | chr21 | 43194268 | 43194914 |
| chr12 | 2616422 | 2617269 | chr2 | 69305805 | 69306166 | chr3 | 190641051 | 190642620 |
| chr17 | 28507701 | 28508491 | chr16 | 26348431 | 26350244 | chr9 | 38488202 | 38488470 |
| chr11 | 43268424 | 43270142 | chr10 | 76307777 | 76309571 | chr11 | 128487466 | 128488235 |
| chr19 | 49165695 | 49166942 | chr19 | 45765048 | 45765704 | chr22 | 48773640 | 48774653 |
| chr8 | 73370450 | 73371076 | chr14 | 103538821 | 103540181 | chr5 | 497406 | 498025 |
| chr2 | 240519761 | 240520278 | chr1 | 18705925 | 18706635 | chr22 | 48427342 | 48427994 |
| chr3 | 197022559 | 197024619 | chr7 | 145784330 | 145785184 | chr1 | 2916910 | 2917270 |
| chr16 | 1535379 | 1536596 | chr2 | 89793078 | 89794475 | chr10 | 129176029 | 129176592 |
| chr9 | 35406202 | 35407017 | chr7 | 47217563 | 47219105 | chr7 | 155358226 | 155358922 |
| chr14 | 101570051 | 101572986 | chr13 | 111400934 | 111401698 | chr20 | 59614042 | 59614872 |
| chr21 | 43985933 | 43986925 | chr13 | 30824092 | 30824983 | chr12 | 32963921 | 32967933 |
| chr20 | 2808655 | 2809485 | chr22 | 20979842 | 20980792 | chr22 | 26912633 | 26913442 |
| chr17 | 48601652 | 48602354 | chr6 | 42145845 | 42146810 | chr17 | 63435721 | 63436195 |
| chr13 | 113930972 | 113931410 | chr5 | 42952614 | 42953779 | chr15 | 41496043 | 41496327 |
| chr16 | 68738268 | 68738663 | chr4 | 7217415 | 7218084 | chr17 | 21775141 | 21775948 |
| chr7 | 47680230 | 47680901 | chr9 | 108473544 | 108474695 | chr22 | 37103211 | 37103610 |
| chr20 | 661852 | 663438 | chr20 | 56702185 | 56702980 | chr8 | 11348959 | 11349665 |
| chr6 | 161835293 | 161835833 | chr6 | 34094317 | 34095229 | chr2 | 120485866 | 120486463 |
| chr7 | 28408248 | 28408775 | chr2 | 74011 | 75546 | chr3 | 129918002 | 129918648 |
| chr3 | 157119992 | 157120598 | chr1 | 245441028 | 245441605 | chr20 | 64092064 | 64092616 |
| chr18 | 26133632 | 26134120 | chr16 | 1192361 | 1193733 | chr11 | 43935586 | 43936237 |
| chr11 | 117907231 | 117907483 | chr14 | 99258148 | 99259243 | chr22 | 20129057 | 20129713 |
| chr7 | 1385713 | 1386474 | chr5 | 3764020 | 3764777 | chr4 | 155421839 | 155422500 |
| chr15 | 99435105 | 99435572 | chr1 | 231233646 | 231234614 | chr10 | 132297435 | 132297988 |
| chr11 | 8339439 | 8340072 | chr3 | 73294531 | 73295689 | chr5 | 135545753 | 135546449 |
| chr11 | 58963346 | 58964057 | chr14 | 104587010 | 104588099 | chr7 | 56477029 | 56477544 |
| chr2 | 94858430 | 94859794 | chr17 | 20841406 | 20842302 | chr1 | 18200792 | 18201457 |
| chr19 | 49195917 | 49197108 | chr11 | 131413925 | 131416915 | chr19 | 29421290 | 29421903 |
| chr3 | 137509463 | 137510228 | chr20 | 45088330 | 45089268 | chr5 | 72036526 | 72039967 |
| chr7 | 39833863 | 39834291 | chr7 | 953068 | 953767 | chr20 | 49721799 | 49722517 |
| chr12 | 130450633 | 130452613 | chr11 | 134502902 | 134503404 | chr16 | 26916294 | 26917230 |
| chr8 | 118108058 | 118110173 | chr11 | 130415929 | 130416221 | chr20 | 25858063 | 25858560 |
| chr12 | 119179322 | 119181333 | chr19 | 1290777 | 1291625 | chr5 | 6353444 | 6354016 |
| chr17 | 80443989 | 80444747 | chr1 | 115479320 | 115479601 | chr11 | 67031030 | 67031672 |
| chr5 | 501173 | 501797 | chr14 | 23179531 | 23181881 | chr1 | 226134772 | 226135401 |
| chr1 | 1291724 | 1292301 | chr4 | 8707122 | 8707663 | chr7 | 61044358 | 61045174 |
| chr9 | 62851588 | 62857122 | chr6 | 33905060 | 33906987 | chr2 | 119283976 | 119284518 |
| chr9 | 133539812 | 133540384 | chr16 | 977311 | 977580 | chr1 | 30663301 | 30664019 |
| chr6 | 3771303 | 3772199 | chr16 | 86569939 | 86571584 | chr9 | 128257150 | 128258166 |
| chr17 | 1905193 | 1906244 | chr18 | 21934296 | 21935587 | chr5 | 178376936 | 178377232 |
| chr5 | 530729 | 531319 | chr2 | 237290442 | 237290847 | chr22 | 24991211 | 24992541 |
| chr13 | 91399570 | 91400232 | chr9 | 130266501 | 130267095 | chr16 | 85330446 | 85330721 |
| chr9 | 88278002 | 88278356 | chr10 | 50024392 | 50024845 | chr7 | 16850965 | 16851509 |
| chr3 | 155309691 | 155310287 | chr4 | 61011780 | 61012788 | chr13 | 95089957 | 95091985 |
| chr11 | 33801695 | 33802227 | chr9 | 95752197 | 95752793 | chr13 | 112984950 | 112985492 |
| chr13 | 52844663 | 52845250 | chr11 | 123211606 | 123212146 | chr10 | 69581501 | 69583404 |
| chr19 | 1923081 | 1924860 | chr2 | 107719670 | 107720695 | chr3 | 184562895 | 184563216 |
| chr3 | 185985997 | 185986609 | chr6 | 4401867 | 4402616 | chr1 | 243931355 | 243931884 |
| chr11 | 64002209 | 64002386 | chr17 | 1055748 | 1056714 | chr14 | 106615492 | 106616448 |
| chr1 | 18480733 | 18481711 | chr10 | 122019236 | 122019998 | chr2 | 240911923 | 240912370 |
| chr5 | 139677033 | 139677375 | chr1 | 16188242 | 16189239 | chr1 | 231429930 | 231430333 |
| chr7 | 50400144 | 50400432 | chr6 | 168573786 | 168574399 | chr7 | 31687204 | 31688416 |
| chr21 | 25999829 | 26000426 | chr2 | 168912611 | 168913745 | chr5 | 141375759 | 141376995 |
| chr1 | 29134260 | 29134875 | chr1 | 40132849 | 40133126 | chr17 | 3012764 | 3013379 |
| chr17 | 79019255 | 79020516 | chr11 | 68900295 | 68901212 | chr18 | 21255378 | 21258195 |
| chr4 | 40197924 | 40200687 | chr17 | 2690128 | 2690750 | chr10 | 44260749 | 44261434 |
| chr10 | 127983441 | 127984206 | chr11 | 113182333 | 113182917 | chr4 | 18319313 | 18320232 |
| chr18 | 62611060 | 62611736 | chr7 | 552856 | 553204 | chr10 | 92045587 | 92045958 |
| chr10 | 67386978 | 67387409 | chr3 | 12895452 | 12896195 | chr19 | 50952360 | 50952800 |
| chr19 | 37333931 | 37334993 | chr4 | 103943377 | 103943749 | chr14 | 93752025 | 93753388 |
| chr1 | 196897181 | 196897752 | chr5 | 140070725 | 140074242 | chr6 | 11153623 | 11154757 |
| chr2 | 202149300 | 202149625 | chr1 | 917257 | 917838 | chr2 | 5019832 | 5020670 |
| chr2 | 232419555 | 232420303 | chr10 | 24159595 | 24164243 | chr16 | 31529958 | 31530295 |
| chr8 | 66542094 | 66542514 | chr14 | 90780249 | 90780822 | chr19 | 388986 | 389722 |
| chr11 | 125129418 | 125130067 | chr2 | 120935787 | 120936084 | chr10 | 38825174 | 38829842 |
| chr2 | 89767489 | 89768101 | chr8 | 1856283 | 1856833 | chr16 | 5508872 | 5509683 |
| chr21 | 45248364 | 45249200 | chr11 | 73957280 | 73958264 | chr12 | 171346 | 172112 |
| chr11 | 134471254 | 134471804 | chr7 | 155338498 | 155339241 | chr9 | 137280953 | 137281512 |
| chr20 | 62473265 | 62473875 | chr12 | 130595724 | 130596575 | chr19 | 5479363 | 5480254 |
| chr19 | 33762197 | 33762697 | chr21 | 44579512 | 44580595 | chr7 | 151205354 | 151205905 |
| chr1 | 161420916 | 161422017 | chr1 | 34562327 | 34563846 | chr1 | 2434736 | 2435324 |
| chr2 | 74647609 | 74649435 | chr15 | 53917997 | 53919402 | chr1 | 228532090 | 228532540 |
| chr16 | 86919229 | 86919877 | chr10 | 110744814 | 110746841 | chr21 | 33051364 | 33052171 |
| chr2 | 95525860 | 95526526 | chr1 | 47207966 | 47208838 | chr1 | 3375285 | 3376082 |
| chr8 | 119673077 | 119673395 | chr10 | 102967031 | 102967923 | chr7 | 62340613 | 62341722 |
| chr2 | 16012764 | 16014597 | chr12 | 132451190 | 132451666 | chr16 | 26991568 | 26993087 |
| chr10 | 95295293 | 95295922 | chr2 | 240832572 | 240833133 | chr8 | 2928216 | 2929328 |
| chr9 | 126036956 | 126037386 | chr16 | 86959274 | 86959818 | chr7 | 154281157 | 154283178 |
| chr5 | 1827793 | 1828541 | chr20 | 57019743 | 57020821 | chr17 | 81498895 | 81499292 |
| chr12 | 132322752 | 132323389 | chr15 | 66037031 | 66037883 | chr2 | 105567992 | 105568304 |
| chr11 | 111230831 | 111231104 | chr20 | 56828525 | 56830214 | chr5 | 179352817 | 179353412 |
| chr22 | 19758236 | 19758959 | chr17 | 29163842 | 29164399 | chr14 | 69795496 | 69797094 |
| chr11 | 122904086 | 122904538 | chr1 | 43008868 | 43010417 | chr15 | 78245060 | 78245425 |
| chr10 | 28939482 | 28939959 | chr3 | 23743277 | 23743778 | chr5 | 174773106 | 174774442 |
| chr19 | 21568320 | 21570779 | chr13 | 110371682 | 110371979 | chr16 | 71516216 | 71516585 |
| chr21 | 45294291 | 45294977 | chr20 | 46008996 | 46010133 | chr7 | 150341214 | 150341545 |
| chr20 | 64052442 | 64052862 | chr10 | 116209014 | 116210036 | chr7 | 44226249 | 44226694 |
| chr6 | 166463218 | 166463612 | chr8 | 1854800 | 1855057 | chr1 | 152188808 | 152189474 |
| chr19 | 49689923 | 49690666 | chr13 | 31022136 | 31023455 | chr10 | 12321345 | 12321853 |
| chr14 | 99737130 | 99737476 | chr12 | 125184340 | 125184901 | chr15 | 91296954 | 91298128 |
| chr12 | 2845794 | 2846540 | chr10 | 122040198 | 122042737 | chr17 | 79273708 | 79274103 |
| chr9 | 32383101 | 32383771 | chr11 | 75430958 | 75431525 | chr16 | 34707505 | 34709278 |
| chr22 | 27296343 | 27296856 | chr16 | 983353 | 983979 | chr11 | 71750872 | 71751646 |
| chr21 | 45250962 | 45251625 | chr9 | 659626 | 660852 | chr11 | 2446137 | 2447052 |
| chr5 | 2591774 | 2592528 | chr9 | 134189090 | 134189988 | chr10 | 131357658 | 131358057 |
| chr19 | 5130468 | 5131327 | chr16 | 82966382 | 82967647 | chr4 | 8103024 | 8103823 |
| chr22 | 44753559 | 44756337 | chr8 | 1748418 | 1749243 | chr20 | 60929536 | 60930708 |
| chr15 | 59993210 | 59993940 | chr13 | 27938639 | 27940240 | chr3 | 192411222 | 192414655 |
| chr18 | 76348329 | 76348766 | chr1 | 20729660 | 20730466 | chr16 | 19882350 | 19883071 |
| chr6 | 27422892 | 27423309 | chr5 | 990660 | 991117 | chr19 | 29367019 | 29368137 |
| chr16 | 83926496 | 83927195 | chr17 | 73482251 | 73482882 | chr18 | 75933924 | 75936282 |
| chr11 | 120882925 | 120883807 | chr3 | 42299842 | 42302320 | chr21 | 46124452 | 46125116 |
| chr22 | 46250985 | 46251233 | chr7 | 132351797 | 132352731 | chr19 | 19110741 | 19112329 |
| chr7 | 130140414 | 130140996 | chr3 | 146407649 | 146408412 | chr11 | 67581047 | 67581987 |
| chr1 | 19359761 | 19361592 | chr20 | 23256256 | 23257209 | chr13 | 65843220 | 65843994 |
| chr16 | 583661 | 584560 | chr18 | 75125233 | 75126151 | chr14 | 95011115 | 95012055 |
| chr20 | 58467137 | 58467709 | chr22 | 31625956 | 31627270 | chr17 | 80486990 | 80487859 |
| chr19 | 22052385 | 22053145 | chr18 | 79397691 | 79398204 | chr2 | 3901264 | 3903100 |
| chr15 | 30044759 | 30046034 | chr1 | 17923221 | 17925624 | chr9 | 126772732 | 126773392 |
| chr20 | 21781443 | 21781854 | chr17 | 79136598 | 79137111 | chr11 | 128163853 | 128165383 |
| chr5 | 77850851 | 77851635 | chr17 | 73555399 | 73556360 | chr12 | 461473 | 461754 |
| chr19 | 54010836 | 54012299 | chr2 | 89829369 | 89830129 | chr10 | 132883483 | 132883794 |
| chr17 | 6237785 | 6238605 | chr10 | 93060511 | 93060983 | chr16 | 56102428 | 56102813 |
| chr7 | 2641098 | 2641970 | chr11 | 102807796 | 102808304 | chr1 | 22228217 | 22229430 |
| chr1 | 228372817 | 228373726 | chr17 | 8697444 | 8698861 | chr15 | 23627249 | 23629168 |
| chr17 | 48551862 | 48553409 | chr22 | 35157878 | 35159575 | chr2 | 21275998 | 21276524 |
| chr19 | 1331414 | 1331954 | chr11 | 69471210 | 69472374 | chr7 | 55017969 | 55018482 |
| chr17 | 21679428 | 21681137 | chr9 | 68307231 | 68307327 | chr1 | 2804865 | 2805655 |
| chr17 | 44628247 | 44629004 | chr1 | 175153504 | 175153843 | chr15 | 65049906 | 65050375 |
| chr5 | 178165321 | 178166050 | chr11 | 45021723 | 45022435 | chr12 | 111360728 | 111361468 |
| chr12 | 121535940 | 121536506 | chr16 | 34608129 | 34610714 | chr14 | 69571013 | 69571592 |
| chr8 | 22365953 | 22366527 | chr7 | 67364488 | 67365187 | chr5 | 1922314 | 1922681 |
| chr20 | 57260205 | 57263126 | chr2 | 206491500 | 206494928 | chr6 | 35786917 | 35787116 |
| chr9 | 113564482 | 113565631 | chr2 | 11133415 | 11134150 | chr11 | 44600435 | 44601909 |
| chr9 | 122131291 | 122132118 | chr2 | 100893745 | 100894873 | chr10 | 130367556 | 130368413 |
| chr17 | 21674357 | 21674943 | chr9 | 127973432 | 127973950 | chr4 | 108165097 | 108166190 |
| chr1 | 38029231 | 38029716 | chr10 | 17773079 | 17774206 | chr9 | 135197077 | 135197850 |
| chr11 | 1876829 | 1877810 | chr8 | 144267577 | 144268006 | chr15 | 49702042 | 49702826 |
| chr6 | 44275191 | 44275690 | chr20 | 58054731 | 58055319 | chr18 | 55095989 | 55097791 |
| chr2 | 468945 | 469668 | chr17 | 83100961 | 83101303 | chr1 | 24340809 | 24341531 |
| chr13 | 23695508 | 23696325 | chr14 | 106212991 | 106215512 | chr22 | 19148087 | 19148910 |
| chr3 | 101540886 | 101542127 | chr3 | 24180632 | 24182118 | chr12 | 128797342 | 128799580 |
| chr1 | 244077388 | 244077985 | chr9 | 124270675 | 124271675 | chr1 | 41342083 | 41342801 |
| chr12 | 130650377 | 130651390 | chr10 | 79334707 | 79336123 | chr19 | 45919914 | 45920481 |
| chr6 | 29661411 | 29662755 | chr20 | 38354331 | 38355091 | chr12 | 6913719 | 6914141 |
| chr6 | 1599237 | 1601114 | chr8 | 52149030 | 52151374 | chr14 | 28954248 | 28958797 |
| chr9 | 99259045 | 99259983 | chr6 | 159820397 | 159821316 | chr19 | 40224234 | 40224805 |
| chr16 | 49670218 | 49670846 | chr12 | 124522748 | 124523596 | chr17 | 72352102 | 72352637 |
| chr15 | 96322887 | 96323410 | chr11 | 70611727 | 70612678 | chr17 | 15604595 | 15604900 |
| chr5 | 180161260 | 180162091 | chr2 | 74446250 | 74447093 | chr2 | 178153899 | 178156079 |
| chr21 | 44285384 | 44286324 | chr17 | 4074040 | 4074830 | chr5 | 5890671 | 5892729 |
| chr1 | 183234348 | 183235579 | chr19 | 1504270 | 1505225 | chr2 | 27054161 | 27054796 |
| chr1 | 202009047 | 202010647 | chr9 | 36820989 | 36821849 | chr5 | 174788195 | 174789456 |
| chr8 | 37224809 | 37225873 | chr15 | 66946270 | 66947351 | chr4 | 13549729 | 13550248 |
| chr8 | 11742497 | 11742934 | chr19 | 50758003 | 50758727 | chr6 | 166409227 | 166409747 |
| chr6 | 28822238 | 28823652 | chr17 | 13107749 | 13108235 | chr11 | 70313886 | 70315057 |
| chr7 | 27213759 | 27214374 | chr16 | 86303164 | 86304018 | chr20 | 25866451 | 25867071 |
| chr1 | 44617045 | 44618066 | chr17 | 22412141 | 22413539 | chr17 | 73465197 | 73466930 |
| chr13 | 26872420 | 26873040 | chr11 | 131694350 | 131694833 | chr16 | 74072181 | 74073618 |
| chr19 | 38567875 | 38569286 | chr10 | 24721494 | 24722006 | chr17 | 8127143 | 8127699 |
| chr1 | 110608038 | 110608630 | chr22 | 27554171 | 27555215 | chr15 | 95364166 | 95368398 |
| chr5 | 1146908 | 1147591 | chr11 | 62927549 | 62928447 | chr13 | 112161552 | 112161921 |
| chr18 | 76384529 | 76385164 | chr15 | 70381167 | 70382929 | chr2 | 238964213 | 238965963 |
| chr8 | 69561984 | 69563896 | chr7 | 24758167 | 24758650 | chr17 | 35515044 | 35515889 |
| chr8 | 22550487 | 22551167 | chr18 | 44756496 | 44758178 | chr2 | 109171466 | 109171732 |
| chr9 | 36966503 | 36967099 | chr3 | 71786087 | 71787432 | chr16 | 86396530 | 86396997 |
| chr2 | 26728372 | 26728937 | chr15 | 93331770 | 93333017 | chr2 | 12720488 | 12722321 |
| chr9 | 136059802 | 136060198 | chr12 | 68487719 | 68488153 | chr1 | 168387313 | 168387875 |
| chr7 | 25894425 | 25895284 | chr3 | 196216573 | 196217136 | chr9 | 97078545 | 97078727 |
| chr20 | 63138195 | 63138502 | chr16 | 81123410 | 81124129 | chr12 | 7872411 | 7873039 |
| chr12 | 5480936 | 5481986 | chr3 | 127990603 | 127991033 | chr6 | 28805293 | 28807130 |
| chr6 | 78629177 | 78629859 | chr17 | 42798970 | 42799417 | chr12 | 52097621 | 52099263 |
| chr20 | 63454740 | 63455438 | chr10 | 100787188 | 100788018 | chr1 | 144554620 | 144555752 |
| chr11 | 134032303 | 134033045 | chr19 | 42108175 | 42110691 | chr14 | 48011421 | 48011753 |
| chr7 | 155405462 | 155405926 | chr20 | 38649542 | 38650721 | chr1 | 3689498 | 3690596 |
| chr5 | 176797459 | 176798119 | chr17 | 21681984 | 21682380 | chr1 | 227559684 | 227560808 |
| chr6 | 27678898 | 27679376 | chr5 | 61816262 | 61817361 | chr16 | 73000532 | 73000995 |
| chr6 | 27680496 | 27680834 | chr5 | 152404499 | 152404791 | chr4 | 7034353 | 7035229 |
| chr6 | 761235 | 761658 | chr1 | 8202044 | 8202791 | chr18 | 79484938 | 79485645 |
| chr10 | 653886 | 654516 | chr16 | 83942961 | 83943759 | chr17 | 41547723 | 41548540 |
| chr3 | 12553882 | 12555142 | chr7 | 43456308 | 43456447 | chr20 | 25987153 | 25987684 |
| chr11 | 73645833 | 73646476 | chr19 | 6003062 | 6004197 | chr4 | 152206346 | 152206936 |
| chr20 | 34549307 | 34549876 | chr10 | 96822733 | 96824024 | chr2 | 206263308 | 206263809 |
| chr7 | 150973568 | 150974157 | chr10 | 132385914 | 132386489 | chr12 | 45065002 | 45065566 |
| chr9 | 88006584 | 88007291 | chr10 | 8279424 | 8280456 | chr2 | 238841254 | 238842677 |
| chr5 | 176739073 | 176739868 | chr1 | 27031950 | 27032523 | chr9 | 134377126 | 134377749 |
| chr16 | 90008112 | 90008985 | chr5 | 4207075 | 4207698 | chr3 | 50501909 | 50502365 |
| chr19 | 1691110 | 1691482 | chr20 | 3898262 | 3899154 | chr22 | 49164294 | 49164575 |
| chr2 | 23663402 | 23664017 | chr2 | 1855263 | 1855588 | chr11 | 121237438 | 121237883 |
| chr18 | 11328298 | 11329732 | chr6 | 27272690 | 27273239 | chr20 | 46200637 | 46201256 |
| chr6 | 2936092 | 2936170 | chr3 | 137808104 | 137808507 | chr7 | 155818420 | 155819063 |
| chr1 | 3249731 | 3250644 | chr15 | 27155634 | 27157460 | chr13 | 93454795 | 93457624 |
| chr17 | 39707511 | 39707828 | chr21 | 10675648 | 10677061 | chr12 | 116455051 | 116456323 |
| chr2 | 109130176 | 109130793 | chr15 | 82098716 | 82099229 | chr15 | 66151316 | 66152198 |
| chr20 | 31566956 | 31568339 | chr5 | 42970597 | 42971414 | chr19 | 27670161 | 27672396 |
| chr4 | 4668974 | 4669455 | chr2 | 89781854 | 89782554 | chr20 | 31637439 | 31638049 |
| chr2 | 240200509 | 240200994 | chr15 | 88115487 | 88115920 | chr16 | 85527060 | 85527766 |
| chr17 | 81409474 | 81409950 | chr5 | 172660250 | 172660801 | chr15 | 29589600 | 29590241 |
| chr17 | 73592595 | 73593041 | chr19 | 50662144 | 50662564 | chr3 | 71708584 | 71710454 |
| chr10 | 11439 | 11502 | chr6 | 167797221 | 167797921 | chr2 | 238845712 | 238846116 |
| chr6 | 30451715 | 30452699 | chr20 | 61223859 | 61224995 | chr19 | 30477547 | 30478742 |
| chr2 | 89585751 | 89586303 | chr17 | 77793107 | 77793689 | chr20 | 43040912 | 43043602 |
| chr10 | 92419915 | 92420455 | chr16 | 6913097 | 6913721 | chr1 | 228072849 | 228073562 |
| chr4 | 3732763 | 3733364 | chr21 | 43359208 | 43359939 | chr2 | 78978921 | 78979623 |
| chr17 | 40863109 | 40863410 | chr12 | 131383991 | 131385203 | chr7 | 60933432 | 60934033 |
| chr20 | 49562280 | 49562774 | chr15 | 32730369 | 32731085 | chr9 | 122220244 | 122221203 |
| chr2 | 54289850 | 54290255 | chr2 | 70655991 | 70657804 | chr16 | 85953428 | 85953720 |
| chr19 | 50514306 | 50515157 | chr15 | 98440909 | 98441827 | chr4 | 41146676 | 41147029 |
| chr22 | 50034212 | 50035563 | chr4 | 6389056 | 6389658 | chr1 | 47241102 | 47241658 |
| chr2 | 240992797 | 240993458 | chr10 | 26143052 | 26143326 | chr9 | 133458767 | 133459101 |
| chr2 | 89611350 | 89611867 | chr17 | 55640492 | 55642628 | chr10 | 127537445 | 127538202 |
| chr12 | 54146285 | 54147050 | chr8 | 54442280 | 54444143 | chr20 | 57570254 | 57570963 |
| chr15 | 93064281 | 93065063 | chr5 | 6581661 | 6582562 | chr20 | 42949957 | 42951148 |
| chr5 | 157651339 | 157651743 | chr1 | 157195128 | 157195654 | chr22 | 39657130 | 39657773 |
| chr17 | 36768468 | 36769218 | chr16 | 89301526 | 89302235 | chr17 | 82064659 | 82065039 |
| chr11 | 32425901 | 32426560 | chr8 | 142041689 | 142042482 | chr14 | 68655289 | 68655866 |
| chr6 | 28982671 | 28983464 | chr16 | 62007069 | 62008604 | chr16 | 87647874 | 87648655 |
| chr17 | 18201494 | 18202088 | chr14 | 78296589 | 78298965 | chr12 | 131003558 | 131004330 |
| chr3 | 32819453 | 32819827 | chr16 | 89088365 | 89089444 | chr13 | 36675078 | 36675497 |
| chr22 | 25028545 | 25028997 | chr20 | 17634920 | 17635499 | chr2 | 46300258 | 46301438 |
| chr17 | 7712237 | 7712931 | chr22 | 39600399 | 39601361 | chr7 | 99598165 | 99598598 |
| chr20 | 64160492 | 64161267 | chr19 | 17697832 | 17699563 | chr2 | 114801772 | 114804554 |
| chr17 | 82005029 | 82005547 | chr15 | 63056377 | 63058254 | chr20 | 31228668 | 31231648 |
| chr14 | 93017229 | 93017665 | chr14 | 55241086 | 55242017 | chr13 | 102731737 | 102733417 |
| chr1 | 159937652 | 159938559 | chr12 | 5086803 | 5087935 | chr10 | 132932429 | 132932871 |
| chr6 | 30447712 | 30450403 | chr2 | 240672963 | 240673638 | chr16 | 1073273 | 1073724 |
| chr1 | 247905247 | 247905786 | chr19 | 601800 | 602622 | chr2 | 14943986 | 14945236 |
| chr12 | 72329985 | 72330839 | chr3 | 52149808 | 52149942 | chr2 | 2581444 | 2581644 |
| chr1 | 24112080 | 24112514 | chr16 | 30452830 | 30453444 | chr12 | 34320248 | 34320611 |
| chr10 | 128709441 | 128710755 | chr5 | 81353406 | 81355711 | chr4 | 6228069 | 6229124 |
| chr22 | 48053558 | 48054278 | chr20 | 38467720 | 38468077 | chr8 | 137335928 | 137337353 |
| chr9 | 133368371 | 133368986 | chr13 | 106098861 | 106100883 | chr17 | 76920256 | 76920747 |
| chr18 | 12254800 | 12255295 | chr3 | 70181526 | 70183354 | chr16 | 6093873 | 6097887 |
| chr11 | 75428085 | 75429083 | chr1 | 1239191 | 1239492 | chr12 | 125689851 | 125690402 |
| chr8 | 142250363 | 142251326 | chr4 | 16907963 | 16909550 | chr12 | 128024097 | 128024705 |
| chr5 | 180170455 | 180170825 | chr8 | 143275504 | 143276990 | chr12 | 130479645 | 130480372 |
| chr2 | 20242061 | 20242694 | chr1 | 24927023 | 24928107 | chr12 | 80717111 | 80717941 |
| chr19 | 50549279 | 50549981 | chr3 | 129383584 | 129385105 | chr2 | 24174804 | 24175107 |
| chr16 | 281153 | 282116 | chr5 | 2914479 | 2915731 | chr16 | 49640097 | 49640843 |
| chr10 | 7444737 | 7445719 | chr14 | 100727808 | 100728500 | chr12 | 109734708 | 109735164 |
| chr17 | 50476586 | 50478106 | chr19 | 1230859 | 1232104 | chr11 | 120182420 | 120183826 |
| chr6 | 7468540 | 7468905 | chr21 | 36946151 | 36947507 | chr18 | 73900582 | 73901789 |
| chr2 | 237005474 | 237005613 | chr15 | 26911580 | 26912973 | chr7 | 145417327 | 145418694 |
| chr12 | 130522129 | 130524198 | chr2 | 94270494 | 94272158 | chr15 | 89391917 | 89392512 |
| chr5 | 1014915 | 1015325 | chr18 | 76203584 | 76205132 | chr5 | 175466523 | 175468683 |
| chr19 | 6710674 | 6711189 | chr17 | 42369896 | 42370903 | chr19 | 13933473 | 13934102 |
| chr12 | 54131863 | 54132913 | chr22 | 44634678 | 44635941 | chr3 | 125694895 | 125695352 |
| chr19 | 52945863 | 52947109 | chr11 | 125351233 | 125351757 | chr6 | 166010290 | 166011101 |
| chr2 | 188606431 | 188606958 | chr2 | 239745869 | 239746207 | chr5 | 135107099 | 135107644 |
| chr12 | 124318335 | 124319097 | chr1 | 22163235 | 22163835 | chr21 | 9751892 | 9753158 |
| chr9 | 112889986 | 112890574 | chr16 | 629348 | 630514 | chr20 | 32605241 | 32605831 |
| chr14 | 104223260 | 104223933 | chr19 | 18396243 | 18396778 | chr11 | 1219857 | 1220231 |
| chr6 | 28478553 | 28479571 | chr1 | 36824156 | 36825135 | chr1 | 208786935 | 208789780 |
| chr7 | 48454765 | 48455270 | chr8 | 46220470 | 46221029 | chr2 | 236272699 | 236274687 |
| chr12 | 132600754 | 132601378 | chr9 | 136346123 | 136346402 | chr1 | 6171007 | 6171528 |
| chr7 | 140673794 | 140674346 | chr11 | 70825989 | 70826314 | chr19 | 54337385 | 54337744 |
| chr19 | 10766249 | 10767372 | chr22 | 44577534 | 44578206 | chr20 | 21096088 | 21096770 |
| chr5 | 33937289 | 33939556 | chr2 | 1575190 | 1575665 | chr6 | 41745528 | 41746055 |
| chr4 | 62594344 | 62595575 | chr10 | 1623973 | 1624578 | chr16 | 34949611 | 34950106 |
| chr10 | 71191193 | 71191960 | chr19 | 1723739 | 1724033 | chr18 | 77759435 | 77760734 |
| chr16 | 57639155 | 57639621 | chr19 | 42174118 | 42175763 | chr10 | 121721384 | 121722440 |
| chr19 | 45160683 | 45162249 | chr10 | 12613265 | 12613873 | chr19 | 50739741 | 50740519 |
| chr9 | 133978737 | 133979782 | chr11 | 63128221 | 63129457 | chr12 | 34401441 | 34402278 |
| chr9 | 134768516 | 134768937 | chr3 | 126994929 | 126995672 | chr10 | 1403566 | 1403977 |
| chr9 | 68318313 | 68319000 | chr17 | 42877786 | 42878424 | chr7 | 61025569 | 61026611 |
| chr1 | 7912690 | 7913436 | chr21 | 10709759 | 10710154 | chr7 | 151199160 | 151199536 |
| chr22 | 20551287 | 20551973 | chr20 | 49395075 | 49395545 | chr18 | 76592990 | 76593899 |
| chr22 | 49304450 | 49305082 | chr3 | 195263469 | 195264496 | chr7 | 50528348 | 50529214 |
| chr8 | 2811299 | 2811924 | chr1 | 180935189 | 180935779 | chr7 | 73627529 | 73628199 |
| chr20 | 61317563 | 61318436 | chr7 | 69232447 | 69234156 | chr17 | 36678776 | 36679395 |
| chr10 | 132407999 | 132408738 | chr2 | 150424697 | 150425673 | chr1 | 226945032 | 226945867 |
| chr5 | 172082173 | 172082791 | chr7 | 4126803 | 4127843 | chr17 | 77450100 | 77450548 |
| chr22 | 17397485 | 17397749 | chr17 | 3673774 | 3674708 | chr6 | 44233178 | 44234963 |
| chr17 | 81523684 | 81524240 | chr1 | 211520512 | 211521085 | chr22 | 29715344 | 29715725 |
| chr19 | 43353709 | 43354200 | chr15 | 74798079 | 74798720 | chr17 | 78272677 | 78273283 |
| chr1 | 46516324 | 46517888 | chr4 | 3807886 | 3808912 | chr20 | 63463525 | 63464353 |
| chr5 | 110956263 | 110956837 | chr10 | 15472647 | 15473936 | chr16 | 86776258 | 86777354 |
| chr19 | 15862602 | 15863024 | chr19 | 37403743 | 37403963 | chr11 | 68890670 | 68891630 |
| chr6 | 19853350 | 19853663 | chr9 | 14993318 | 14993689 | chr1 | 2687341 | 2688297 |
| chr18 | 21240561 | 21241229 | chr19 | 33400929 | 33401344 | chr7 | 42090938 | 42091596 |
| chr20 | 38343005 | 38343703 | chr11 | 132605285 | 132606864 | chr16 | 1098423 | 1098956 |
| chr10 | 117125853 | 117126373 | chr16 | 405072 | 405651 | chr7 | 1633124 | 1633882 |
| chr19 | 50722370 | 50723101 | chr19 | 9960511 | 9960881 | chr13 | 66314229 | 66315761 |
| chr12 | 130480844 | 130481485 | chr6 | 169251906 | 169252320 | chr2 | 236988893 | 236989672 |
| chr17 | 28415398 | 28416494 | chr1 | 6207118 | 6207746 | chr4 | 80207077 | 80207818 |
| chr16 | 86733600 | 86735071 | chr16 | 68406587 | 68407243 | chr2 | 219313099 | 219313617 |
| chr6 | 34514501 | 34515375 | chr6 | 138572056 | 138573002 | chr1 | 7149606 | 7150127 |
| chr16 | 1184303 | 1185103 | chr19 | 11416812 | 11417440 | chr19 | 28146410 | 28148107 |
| chr20 | 63456672 | 63457363 | chr10 | 11863482 | 11864424 | chr14 | 101496169 | 101496784 |
| chr15 | 100849145 | 100849677 | chr21 | 9822551 | 9822654 | chr2 | 1767240 | 1767691 |
| chr1 | 154867198 | 154868039 | chr22 | 49850148 | 49851326 | chr18 | 73866949 | 73868445 |
| chr6 | 28720295 | 28721226 | chr7 | 70631969 | 70632553 | chr1 | 56439379 | 56441165 |
| chr12 | 130491198 | 130491971 | chr7 | 155383050 | 155384081 | chr1 | 112174810 | 112177634 |
| chr17 | 44374568 | 44375382 | chr22 | 31968918 | 31969600 | chr5 | 174459532 | 174460273 |
| chr19 | 2717237 | 2718054 | chr13 | 110811371 | 110812324 | chr22 | 46477073 | 46477794 |
| chr15 | 66024796 | 66026263 | chr21 | 36668663 | 36669430 | chr3 | 72304034 | 72305359 |
| chr5 | 171319230 | 171320844 | chr17 | 81388628 | 81390136 | chr20 | 57735865 | 57736926 |
| chr19 | 54966086 | 54967254 | chr18 | 3155139 | 3156561 | chr5 | 73183382 | 73184751 |
| chr17 | 17722292 | 17723204 | chr16 | 36236612 | 36238081 | chr1 | 3609016 | 3609376 |
| chr1 | 148354010 | 148355291 | chr11 | 119782024 | 119782372 | chr8 | 11644241 | 11644774 |
| chr14 | 93995717 | 93996280 | chr8 | 43539677 | 43541491 | chr11 | 126557546 | 126559113 |
| chr17 | 74268468 | 74268908 | chr22 | 50014165 | 50014534 | chr9 | 130943372 | 130944760 |
| chr4 | 1841360 | 1841970 | chr1 | 5601392 | 5601791 | chr17 | 81527988 | 81528595 |
| chr17 | 58196972 | 58197237 | chr19 | 46494897 | 46495976 | chr6 | 51213949 | 51214826 |
| chr12 | 119153274 | 119154623 | chr12 | 129537681 | 129539300 | chr10 | 37137342 | 37137752 |
| chr10 | 128927810 | 128928577 | chr16 | 87897415 | 87898020 | chr17 | 79521943 | 79522668 |
| chr12 | 132227635 | 132228099 | chr10 | 121595506 | 121596142 | chr18 | 47667773 | 47668542 |
| chr15 | 59904576 | 59905545 | chr20 | 63251490 | 63252039 | chr1 | 46967596 | 46969405 |
| chr2 | 120879507 | 120881074 | chr10 | 2543425 | 2543900 | chr2 | 27753244 | 27754400 |
| chr22 | 29432219 | 29432979 | chr8 | 4469359 | 4470122 | chr4 | 183558682 | 183559174 |
| chr1 | 29990938 | 29991766 | chr19 | 5893380 | 5894098 | chr10 | 113553407 | 113554771 |
| chr7 | 151459514 | 151460282 | chr5 | 475609 | 476417 | chr16 | 2710951 | 2711440 |
| chr16 | 85480019 | 85480624 | chr12 | 132558342 | 132558835 | chr4 | 76867424 | 76868217 |
| chr12 | 122659655 | 122660233 | chr10 | 46053939 | 46056094 | chr3 | 170975553 | 170976447 |
| chr10 | 618757 | 619055 | chr7 | 62401185 | 62401732 | chr2 | 23658545 | 23658870 |
| chr12 | 52250199 | 52250540 | chr1 | 201268639 | 201269029 | chr20 | 3658430 | 3659899 |
| chr16 | 73482855 | 73483624 | chr22 | 33302376 | 33302909 | chr5 | 141426004 | 141426849 |
| chr16 | 87881340 | 87882053 | chr5 | 130645727 | 130647007 | chr3 | 180869815 | 180870513 |
| chr22 | 43225917 | 43226738 | chr12 | 114734154 | 114734606 | chr10 | 3238649 | 3239460 |
| chr6 | 20068526 | 20069655 | chr14 | 29928258 | 29928944 | chr10 | 48823747 | 48826788 |
| chr11 | 3579512 | 3579985 | chr5 | 139902162 | 139903467 | chr17 | 39867762 | 39868800 |
| chr12 | 95549846 | 95551835 | chr11 | 64003466 | 64004048 | chr6 | 169095439 | 169096628 |
| chr6 | 26017152 | 26019604 | chr6 | 157236819 | 157237059 | chr16 | 85696974 | 85697473 |
| chr11 | 3205265 | 3206482 | chr12 | 32390959 | 32391243 | chr5 | 181041260 | 181042460 |
| chr18 | 45838404 | 45839402 | chr17 | 61505997 | 61508020 | chr8 | 17780384 | 17782950 |
| chr17 | 10311801 | 10312716 | chr9 | 115476801 | 115478830 | chr5 | 88539021 | 88540536 |
| chr3 | 156120734 | 156120930 | chr16 | 80387354 | 80387750 | chr19 | 30779118 | 30780275 |
| chr10 | 130045014 | 130045584 | chr15 | 93641686 | 93641830 | chr12 | 118754763 | 118758138 |
| chr16 | 3442828 | 3444131 | chr7 | 63241671 | 63246098 | chr9 | 111604031 | 111604538 |
| chr17 | 49907142 | 49909772 | chr20 | 49583692 | 49584378 | chr11 | 126815634 | 126815947 |
| chr18 | 36188227 | 36189116 | chr8 | 41651301 | 41652101 | chr15 | 79195742 | 79196811 |
| chr17 | 66303720 | 66304175 | chr15 | 74227909 | 74228748 | chr10 | 1545661 | 1546441 |
| chr20 | 2779445 | 2780925 | chr22 | 42304036 | 42304691 | chr5 | 857552 | 858001 |
| chr10 | 42103229 | 42103950 | chr20 | 48630041 | 48630925 | chr10 | 63033608 | 63033851 |
| chr4 | 79049733 | 79050430 | chr3 | 13019223 | 13019629 | chr18 | 37330164 | 37330951 |
| chr1 | 151710165 | 151710735 | chr5 | 1413168 | 1413788 | chr6 | 149979036 | 149980423 |
| chr8 | 37724921 | 37726067 | chr4 | 2021547 | 2022139 | chr4 | 7758166 | 7758620 |
| chr21 | 34524775 | 34525456 | chr10 | 133525102 | 133526019 | chr12 | 109621806 | 109622119 |
| chr14 | 70820861 | 70821648 | chr3 | 186193218 | 186194627 | chr4 | 170003995 | 170004969 |
| chr6 | 73394415 | 73395213 | chr8 | 46189053 | 46190075 | chr6 | 29802836 | 29806327 |
| chr16 | 54651509 | 54652427 | chr10 | 58327042 | 58327352 | chr17 | 46822058 | 46822622 |
| chr2 | 44975667 | 44976150 | chr2 | 9982985 | 9984069 | chr19 | 45917556 | 45918296 |
| chr1 | 29439340 | 29440036 | chr13 | 113791986 | 113792594 | chr21 | 43361485 | 43361839 |
| chr8 | 142621728 | 142622405 | chr6 | 40310895 | 40311543 | chr1 | 2846396 | 2846890 |
| chr7 | 155489973 | 155490499 | chr15 | 28315245 | 28315736 | chr2 | 136406781 | 136408031 |
| chr22 | 27065361 | 27066007 | chr20 | 63290819 | 63291303 | chr15 | 27679397 | 27680409 |
| chr4 | 6761093 | 6762525 | chr2 | 239363975 | 239364239 | chr10 | 122487194 | 122487766 |
| chr19 | 36257672 | 36258743 | chr17 | 20030958 | 20031510 | chr7 | 149415528 | 149415923 |
| chr2 | 43100594 | 43101482 | chr17 | 80191890 | 80192539 | chr15 | 42079452 | 42079834 |
| chr12 | 105500647 | 105501011 | chr2 | 66443828 | 66444664 | chr5 | 179362389 | 179362622 |
| chr19 | 9697441 | 9698614 | chr1 | 2043123 | 2043979 | chr1 | 236727379 | 236727796 |
| chr1 | 148317364 | 148317864 | chr9 | 133251614 | 133252011 | chr15 | 76191924 | 76192527 |
| chr2 | 129765540 | 129766226 | chr17 | 81118050 | 81119008 | chr5 | 174132933 | 174133699 |
| chr6 | 156616994 | 156619466 | chr8 | 25983439 | 25984163 | chr17 | 72132324 | 72133984 |
| chr9 | 135984279 | 135985167 | chr5 | 17058797 | 17059848 | chr8 | 46414708 | 46415469 |
| chr10 | 5582052 | 5583951 | chr1 | 44203060 | 44205371 | chr8 | 141701319 | 141702834 |
| chr1 | 3424214 | 3424765 | chr6 | 27292888 | 27293983 | chr16 | 89658830 | 89659228 |
| chr6 | 41528110 | 41528452 | chr4 | 89489871 | 89490229 | chr15 | 39809487 | 39809973 |
| chr1 | 203350351 | 203351605 | chr4 | 153576195 | 153578168 | chr1 | 228446724 | 228447111 |
| chr19 | 7527187 | 7528257 | chr19 | 2741015 | 2742778 | chr13 | 52608995 | 52610029 |
| chr8 | 48419386 | 48420014 | chr7 | 31372327 | 31372969 | chr22 | 22616198 | 22617918 |
| chr1 | 2683391 | 2684305 | chr6 | 71421033 | 71422224 | chr17 | 73533199 | 73533760 |
| chr17 | 21763807 | 21764363 | chr22 | 18048246 | 18048702 | chr1 | 30657887 | 30658981 |
| chr15 | 67104531 | 67105458 | chr8 | 106447762 | 106448314 | chr5 | 181126684 | 181127095 |
| chr20 | 22558309 | 22559385 | chr16 | 85234735 | 85235157 | chr6 | 166005045 | 166005527 |
| chr2 | 1643391 | 1643544 | chr16 | 89640660 | 89641426 | chr1 | 20301394 | 20301994 |
| chr9 | 16103045 | 16103986 | chr14 | 101442416 | 101442746 | chr8 | 11008138 | 11009315 |
| chr12 | 130716918 | 130718434 | chr22 | 38304894 | 38306289 | chr18 | 22219489 | 22220120 |
| chr19 | 55489415 | 55489578 | chr8 | 46231926 | 46233007 | chr11 | 1391575 | 1392120 |
| chr12 | 34334783 | 34336071 | chr12 | 55024818 | 55027350 | chr19 | 15737356 | 15739564 |
| chr7 | 232964 | 234431 | chr20 | 56914675 | 56915946 | chr5 | 317041 | 317728 |
| chr13 | 20394418 | 20395103 | chr1 | 43312181 | 43312644 | chr7 | 153943263 | 153944282 |
| chr19 | 14265396 | 14266280 | chr2 | 3779322 | 3780362 | chr7 | 1575885 | 1577244 |
| chr1 | 35120606 | 35121084 | chr12 | 74111758 | 74113851 | chr10 | 84214287 | 84215055 |
| chr12 | 5648388 | 5649241 | chr3 | 66798184 | 66798560 | chr1 | 64323418 | 64324116 |
| chr1 | 156881253 | 156881883 | chr11 | 79506765 | 79508224 | chr1 | 204315309 | 204315867 |
| chr7 | 114944844 | 114945483 | chr9 | 124399645 | 124400363 | chr4 | 114004628 | 114004878 |
| chr2 | 16166453 | 16166814 | chr2 | 241088552 | 241089123 | chr8 | 1163712 | 1164635 |
| chr19 | 38082549 | 38082928 | chr3 | 197659554 | 197659933 | chr12 | 119235101 | 119237245 |
| chr20 | 45303926 | 45305033 | chr8 | 29806740 | 29807449 | chr12 | 34380758 | 34381407 |
| chr17 | 72123327 | 72123778 | chr20 | 62380461 | 62381593 | chr14 | 95870319 | 95871190 |
| chr6 | 27669453 | 27670214 | chr3 | 156605788 | 156606254 | chr8 | 10798069 | 10799416 |
| chr2 | 21100560 | 21101933 | chr10 | 101132969 | 101133582 | chr4 | 8649250 | 8649506 |
| chr14 | 96736641 | 96737349 | chr10 | 1195718 | 1197664 | chr19 | 31108280 | 31108994 |
| chr9 | 126665763 | 126667325 | chr1 | 154346393 | 154346992 | chr12 | 14708238 | 14710609 |
| chr6 | 107112572 | 107113840 | chr20 | 41387475 | 41387928 | chr1 | 184191254 | 184192159 |
| chr8 | 143399905 | 143401216 | chr7 | 31137048 | 31137383 | chr10 | 37747184 | 37749327 |
| chr1 | 240770872 | 240771685 | chr16 | 83727113 | 83728750 | chr2 | 236715361 | 236715850 |
| chr3 | 156751108 | 156751873 | chr20 | 56132746 | 56136017 | chr1 | 4600033 | 4600590 |
| chr9 | 65736870 | 65737823 | chr17 | 1947172 | 1947804 | chr6 | 166757596 | 166758557 |
| chr13 | 110388087 | 110388979 | chr20 | 62580915 | 62581652 | chr22 | 46735544 | 46736778 |
| chr16 | 50287901 | 50288306 | chr21 | 41045173 | 41046103 | chr7 | 23904302 | 23904479 |
| chr11 | 59107524 | 59108179 | chr17 | 74439941 | 74440492 | chr10 | 101558810 | 101559453 |
| chr9 | 62849903 | 62850484 | chr20 | 24467969 | 24468757 | chr18 | 10072687 | 10074628 |
| chr19 | 1539900 | 1540529 | chr13 | 25372856 | 25373726 | chr12 | 105950505 | 105952343 |
| chr1 | 68051460 | 68051787 | chr13 | 25069584 | 25069775 | chr9 | 137145877 | 137146689 |
| chr10 | 98025750 | 98026907 | chr9 | 134225650 | 134226096 | chr10 | 97151732 | 97152611 |
| chr12 | 54127594 | 54129826 | chr7 | 115173116 | 115173770 | chr12 | 57737240 | 57740442 |
| chr4 | 41981689 | 41982069 | chr3 | 65954360 | 65956028 | chr21 | 10738139 | 10738675 |
| chr1 | 157107704 | 157108467 | chr10 | 97089291 | 97089785 | chr20 | 50848676 | 50849354 |
| chr8 | 48624555 | 48625254 | chr3 | 126309106 | 126309497 | chr16 | 12090210 | 12090671 |
| chr8 | 95181083 | 95181452 | chr15 | 27956496 | 27957291 | chr19 | 27718961 | 27719774 |
| chr21 | 37709937 | 37711039 | chr17 | 82436649 | 82436976 | chr11 | 126771917 | 126772591 |
| chr20 | 21089788 | 21090920 | chr1 | 47984642 | 47985830 | chr8 | 6540812 | 6541556 |
| chr10 | 131689184 | 131689466 | chr8 | 143311417 | 143313364 | chr8 | 57142294 | 57145106 |
| chr17 | 76273841 | 76274491 | chr2 | 108977460 | 108978062 | chr16 | 34089263 | 34090056 |
| chr21 | 42075751 | 42076154 | chr5 | 66590974 | 66593264 | chr10 | 43230438 | 43230758 |
| chr8 | 893372 | 894381 | chr2 | 148999332 | 149001196 | chr5 | 54524727 | 54525670 |
| chr11 | 125235495 | 125237669 | chr18 | 79800007 | 79800563 | chr19 | 5306753 | 5307102 |
| chr4 | 6324611 | 6325943 | chr3 | 64360503 | 64362427 | chr16 | 34710624 | 34711310 |
| chr5 | 61732224 | 61732997 | chr22 | 48248744 | 48250543 | chr4 | 1415343 | 1416089 |
| chr2 | 16103197 | 16103385 | chr19 | 54434228 | 54436755 | chr14 | 101182551 | 101183613 |
| chr10 | 122462963 | 122463690 | chr16 | 34605639 | 34606403 | chr5 | 174209564 | 174210886 |
| chr11 | 64005175 | 64005633 | chr12 | 52231942 | 52232648 | chr6 | 170066994 | 170067740 |
| chr1 | 148309492 | 148310605 | chr5 | 178442436 | 178442972 | chr10 | 100802360 | 100803020 |
| chr19 | 38452601 | 38453446 | chr12 | 124610411 | 124610871 | chr11 | 69102023 | 69102873 |
| chr20 | 25984843 | 25985481 | chr6 | 2918752 | 2919662 | chr9 | 134230707 | 134231279 |
| chr8 | 139655865 | 139656784 | chr11 | 11979181 | 11979845 | chr7 | 127534904 | 127536432 |
| chr2 | 1254214 | 1255273 | chr8 | 134466713 | 134467095 | chr9 | 126128643 | 126129356 |
| chr4 | 186203514 | 186205051 | chr9 | 132214109 | 132214444 | chr22 | 35007931 | 35009072 |
| chr8 | 143185913 | 143186543 | chr19 | 17641122 | 17641467 | chr11 | 78714985 | 78716155 |
| chr2 | 236688688 | 236689320 | chr2 | 70253076 | 70255416 | chr19 | 3110267 | 3110693 |
| chr12 | 57283464 | 57283977 | chr10 | 76111441 | 76112412 | chr7 | 73330191 | 73330490 |
| chr11 | 69891280 | 69892249 | chr2 | 86339047 | 86340715 | chr16 | 2237817 | 2238163 |
| chr10 | 99330715 | 99331366 | chr17 | 73545254 | 73546676 | chr1 | 25907075 | 25907525 |
| chr11 | 118634537 | 118635635 | chr22 | 39315962 | 39317550 | chr22 | 44585961 | 44586525 |
| chr3 | 133596504 | 133598682 | chr1 | 165284537 | 165284926 | chr12 | 52081364 | 52082434 |
| chr1 | 20580368 | 20581167 | chr4 | 37890049 | 37890281 | chr6 | 158835466 | 158836373 |
| chr15 | 70823164 | 70824272 | chr7 | 73717926 | 73718628 | chr7 | 157955104 | 157955974 |
| chr21 | 33031354 | 33033965 | chr6 | 150623370 | 150623802 | chr5 | 1867545 | 1868331 |
| chr1 | 173703822 | 173706101 | chr7 | 150911165 | 150911647 | chr21 | 45438428 | 45438930 |
| chr7 | 139522247 | 139523144 | chr21 | 40242835 | 40244366 | chr14 | 99029471 | 99030594 |
| chr7 | 51262070 | 51262572 | chr17 | 4591213 | 4591652 | chr11 | 115674101 | 115674656 |
| chr2 | 23595023 | 23596132 | chr2 | 234335703 | 234336188 | chr12 | 7992207 | 7995097 |
| chr16 | 86858495 | 86858915 | chr9 | 135384445 | 135384901 | chr8 | 141548299 | 141548891 |
| chr4 | 8725177 | 8726253 | chr14 | 100563547 | 100564010 | chr14 | 36766627 | 36768077 |
| chr12 | 2834478 | 2835594 | chr16 | 88678394 | 88678783 | chr20 | 38183867 | 38184633 |
| chr2 | 120709413 | 120710324 | chr9 | 132504979 | 132506088 | chr6 | 47376750 | 47377313 |
| chr1 | 225939218 | 225940100 | chr18 | 12776999 | 12777646 | chr11 | 18972311 | 18973010 |
| chr5 | 181170457 | 181171521 | chr19 | 2463002 | 2463545 | chr2 | 197785107 | 197786206 |
| chr11 | 45145173 | 45145583 | chr6 | 27757530 | 27757919 | chr8 | 1846911 | 1848045 |
| chr3 | 72654944 | 72655987 | chr4 | 54276428 | 54277410 | chr15 | 27675007 | 27675911 |
| chr20 | 44480196 | 44480752 | chr9 | 63859523 | 63860174 | chr12 | 131493264 | 131493998 |
| chr6 | 30418022 | 30420417 | chr1 | 147085340 | 147085751 | chr20 | 51565658 | 51566488 |
| chr10 | 86712459 | 86712934 | chr7 | 150900928 | 150901721 | chr9 | 103202884 | 103203968 |
| chr20 | 44356065 | 44356561 | chr9 | 137176204 | 137176788 | chr19 | 54635564 | 54636505 |
| chr17 | 83184104 | 83184542 | chr21 | 13979616 | 13981107 | chr17 | 73343160 | 73343913 |
| chr20 | 59398976 | 59399867 | chr16 | 46404597 | 46405061 | chr19 | 15022899 | 15023316 |
| chr22 | 46510347 | 46510969 | chr10 | 1377482 | 1378220 | chr3 | 51897330 | 51898827 |
| chr12 | 100715963 | 100716859 | chr19 | 45912481 | 45913528 | chr1 | 201288650 | 201289452 |
| chr1 | 148328910 | 148329696 | chr21 | 46138312 | 46138776 | chr8 | 46114341 | 46114902 |
| chr22 | 44670619 | 44671221 | chr10 | 119658317 | 119658624 | chr7 | 151245922 | 151246736 |
| chr5 | 483332 | 483703 | chr2 | 241908758 | 241909291 | chr15 | 77015749 | 77016144 |
| chr1 | 3061942 | 3062539 | chr2 | 219688482 | 219689025 | chr12 | 3367020 | 3367480 |
| chr7 | 158473122 | 158473612 | chr2 | 230990256 | 230991241 | chr5 | 120457895 | 120458347 |
| chr12 | 100553062 | 100554192 | chr8 | 142246584 | 142247073 | chr16 | 81154657 | 81156160 |
| chr3 | 46372952 | 46373965 | chr4 | 3481333 | 3481988 | chr6 | 33758878 | 33759389 |
| chr2 | 142179793 | 142181372 | chr2 | 112140418 | 112140843 | chr20 | 20199481 | 20200048 |
| chr9 | 120386228 | 120386811 | chr11 | 33864310 | 33867025 | chr17 | 40542080 | 40542757 |
| chr1 | 43662436 | 43663152 | chr5 | 139808771 | 139809500 | chr21 | 37281142 | 37282879 |
| chr10 | 97578207 | 97578765 | chr4 | 185050115 | 185051112 | chr4 | 6753539 | 6754349 |
| chr16 | 50592162 | 50593129 | chr8 | 142599338 | 142600146 | chr8 | 78858895 | 78859457 |
| chr17 | 73820929 | 73821410 | chr16 | 34740021 | 34741110 | chr20 | 59769434 | 59770226 |
| chr21 | 46140364 | 46141022 | chr14 | 104545812 | 104546386 | chr3 | 127142027 | 127142568 |
| chr10 | 124733300 | 124733997 | chr13 | 49218511 | 49219304 | chr18 | 14999752 | 15000335 |
| chr9 | 128409330 | 128410181 | chr7 | 37430721 | 37432639 | chr2 | 169768118 | 169768674 |
| chr6 | 32441086 | 32443097 | chr16 | 2152340 | 2153379 | chr9 | 92189553 | 92190099 |
| chr14 | 105193881 | 105195133 | chr1 | 34521176 | 34524376 | chr7 | 57770299 | 57771112 |
| chr7 | 19143404 | 19143925 | chr16 | 56627149 | 56627722 | chr12 | 49624004 | 49625229 |
| chr21 | 44748515 | 44749394 | chr19 | 11710094 | 11710578 | chr19 | 18379797 | 18380475 |
| chr13 | 114026828 | 114027345 | chr14 | 103623962 | 103624489 | chr5 | 141356760 | 141357171 |
| chr3 | 66032641 | 66034174 | chr3 | 26625254 | 26625609 | chr7 | 149071602 | 149072098 |
| chr8 | 22866024 | 22866500 | chr14 | 78248623 | 78249888 | chr16 | 85838433 | 85838717 |
| chr7 | 153888904 | 153889202 | chr7 | 27943340 | 27943699 | chr7 | 157875122 | 157875360 |
| chr6 | 137341773 | 137342944 | chr7 | 149774242 | 149774679 | chr17 | 79358971 | 79360022 |
| chr1 | 114854544 | 114855109 | chr12 | 128765931 | 128767078 | chr18 | 37266181 | 37266585 |
| chr1 | 179815846 | 179816774 | chr20 | 61248424 | 61249168 | chr10 | 3419894 | 3420563 |
| chr6 | 158028493 | 158028978 | chr20 | 62995794 | 62996101 | chr1 | 20492087 | 20492987 |
| chr11 | 69780761 | 69781121 | chr20 | 46214704 | 46216300 | chr2 | 1873788 | 1874704 |
| chr17 | 49924227 | 49924697 | chr8 | 601409 | 601756 | chr14 | 90098778 | 90100292 |
| chr11 | 60615450 | 60615934 | chr7 | 155497442 | 155497770 | chr17 | 77535959 | 77537399 |
| chr3 | 194385989 | 194386442 | chr4 | 178454223 | 178455030 | chr9 | 134944511 | 134945126 |
| chr22 | 26174231 | 26174790 | chr4 | 3852131 | 3852829 | chr21 | 22406896 | 22407778 |
| chr2 | 166609635 | 166611017 | chr5 | 139759257 | 139759814 | chr20 | 53141984 | 53143116 |
| chr2 | 128265303 | 128266597 | chr3 | 196640345 | 196641025 | chr1 | 228465394 | 228465874 |
| chr9 | 2241184 | 2241658 | chr6 | 30206314 | 30207315 | chr19 | 54420229 | 54421141 |
| chr12 | 132282882 | 132283238 | chr17 | 68774407 | 68774987 | chr21 | 9922485 | 9925901 |
| chr17 | 1180717 | 1181678 | chr20 | 57212194 | 57212874 | chr2 | 19864339 | 19864650 |
| chr22 | 29359701 | 29361361 | chr19 | 3123107 | 3124006 | chr3 | 126416291 | 126416633 |
| chr12 | 132672650 | 132672780 | chr16 | 67154202 | 67154863 | chr13 | 29954313 | 29955364 |
| chr9 | 137049234 | 137049861 | chr2 | 206250532 | 206251167 | chr7 | 1033619 | 1034171 |
| chr1 | 20223261 | 20224188 | chr21 | 39788511 | 39789169 | chr7 | 2762535 | 2763064 |
| chr7 | 8389519 | 8391107 | chr4 | 27101011 | 27101499 | chr6 | 170280098 | 170280910 |
| chr4 | 113978800 | 113978974 | chr1 | 210311923 | 210312359 | chr2 | 3912798 | 3913547 |
| chr1 | 1260110 | 1260586 | chr16 | 1108916 | 1109388 | chr2 | 41947690 | 41948212 |
| chr4 | 3489279 | 3489713 | chr14 | 64762486 | 64763427 | chr4 | 6322942 | 6323380 |
| chr6 | 40599058 | 40599958 | chr15 | 29675533 | 29676126 | chr1 | 111904510 | 111904824 |
| chr15 | 45035735 | 45036260 | chr7 | 154892694 | 154893265 | chr17 | 81052529 | 81053358 |
| chr1 | 201283879 | 201284410 | chr2 | 98299381 | 98300833 | chr2 | 2542984 | 2543609 |
| chr10 | 97889513 | 97889879 | chr1 | 10649490 | 10651102 | chr9 | 130316719 | 130317935 |
| chr5 | 179326334 | 179327205 | chr20 | 60956262 | 60958455 | chr1 | 18410639 | 18411236 |
| chr9 | 134229613 | 134229955 | chr15 | 77795687 | 77796125 | chr9 | 131037334 | 131038226 |
| chr1 | 3130612 | 3131442 | chr5 | 176695107 | 176696556 | chr9 | 133610511 | 133611351 |
| chr6 | 28491229 | 28491977 | chr8 | 141586601 | 141587283 | chr19 | 48682993 | 48684010 |
| chr9 | 65427521 | 65429373 | chr8 | 142361745 | 142362168 | chr18 | 78418491 | 78419860 |
| chr12 | 130633487 | 130634346 | chr2 | 43304797 | 43307334 | chr3 | 184338098 | 184338766 |
| chr7 | 1558300 | 1558646 | chr6 | 149484061 | 149485609 | chr8 | 115157383 | 115158641 |
| chr18 | 79005939 | 79006417 | chr9 | 130320397 | 130320913 | chr6 | 1416341 | 1416537 |
| chr22 | 21703792 | 21704057 | chr21 | 10724200 | 10725106 | chr7 | 51181736 | 51182577 |
| chr2 | 38072637 | 38073743 | chr5 | 176664561 | 176665307 | chr1 | 200895632 | 200896077 |
| chr19 | 6659574 | 6660292 | chr2 | 20195738 | 20197127 | chr4 | 27128215 | 27128807 |
| chr1 | 68050646 | 68050809 | chr6 | 37699535 | 37699973 | chr4 | 2804863 | 2805316 |
| chr8 | 143402553 | 143403214 | chr16 | 73283207 | 73284090 | chr8 | 2556940 | 2557732 |
| chr12 | 113425656 | 113426373 | chr1 | 30904139 | 30906161 | chr18 | 7038714 | 7039063 |
| chr5 | 173246490 | 173248404 | chr10 | 87407616 | 87408141 | chr1 | 161427263 | 161428157 |
| chr1 | 3718086 | 3718660 | chr10 | 103555637 | 103556283 | chr1 | 11751967 | 11753011 |
| chr19 | 51167467 | 51168338 | chr7 | 156993106 | 156993662 | chr15 | 22417954 | 22418952 |
| chr1 | 3483934 | 3484348 | chr17 | 35313661 | 35314827 | chr8 | 60413299 | 60413923 |
| chr10 | 121736877 | 121738423 | chr15 | 51342386 | 51342770 | chr20 | 4248243 | 4248571 |
| chr1 | 244139674 | 244140242 | chr7 | 8461475 | 8462343 | chr14 | 104747975 | 104748653 |
| chr6 | 168314962 | 168316247 | chr10 | 26284770 | 26286451 | chr9 | 136917738 | 136918057 |
| chr17 | 73549420 | 73550328 | chr11 | 34912689 | 34913696 | chr12 | 112413353 | 112414674 |
| chr11 | 3222947 | 3223496 | chr15 | 74222005 | 74223132 | chr17 | 73435322 | 73435694 |
| chr10 | 112212058 | 112212565 | chr17 | 36632815 | 36633539 | chr19 | 28889383 | 28890343 |
| chr11 | 117443901 | 117444564 | chr2 | 218873873 | 218875356 | chr11 | 50298381 | 50299042 |
| chr15 | 45376567 | 45377224 | chr13 | 109280191 | 109281883 | chr22 | 45995944 | 45996943 |
| chr7 | 55869140 | 55869978 | chr9 | 130914054 | 130914742 | chr8 | 25600317 | 25601420 |
| chr17 | 8129172 | 8129881 | chr15 | 38074266 | 38076371 | chr2 | 108981993 | 108983173 |
| chr10 | 118246999 | 118248963 | chr9 | 91038033 | 91038371 | chr6 | 110398708 | 110399813 |
| chr8 | 53884800 | 53886211 | chr13 | 106417588 | 106418610 | chr4 | 6359959 | 6360610 |
| chr8 | 28319586 | 28320577 | chr4 | 5953596 | 5954649 | chr16 | 17070664 | 17071091 |
| chr8 | 86926351 | 86929647 | chr14 | 96614512 | 96614940 | chr17 | 79207183 | 79207659 |
| chr5 | 57312746 | 57312987 | chr7 | 288988 | 289349 | chr20 | 19090072 | 19091548 |
| chr12 | 130512569 | 130514657 | chr17 | 29562396 | 29562940 | chr5 | 176725514 | 176726056 |
| chr11 | 12802701 | 12803079 | chr9 | 137173146 | 137173776 | chr14 | 33801021 | 33801332 |
| chr4 | 8202190 | 8203182 | chr1 | 4404522 | 4405902 | chr5 | 176184815 | 176187239 |
| chr21 | 41779108 | 41780209 | chr9 | 122128225 | 122128743 | chr19 | 38976537 | 38976862 |
| chr6 | 108478203 | 108478547 | chr5 | 157608097 | 157608743 | chr10 | 121334191 | 121335984 |
| chr10 | 89208425 | 89209611 | chr8 | 32647074 | 32647447 | chr21 | 13035516 | 13037169 |
| chr20 | 47886506 | 47887036 | chr17 | 44386155 | 44386476 | chr4 | 20003806 | 20004218 |
| chr10 | 97119342 | 97119792 | chr10 | 114714174 | 114716041 | chr11 | 125314214 | 125315057 |
| chr17 | 17528622 | 17529192 | chr19 | 48908169 | 48909542 | chr14 | 105862996 | 105864344 |
| chr4 | 4033240 | 4034583 | chr1 | 17561238 | 17561781 | chr12 | 80513299 | 80513871 |
| chr17 | 40499642 | 40499955 | chr14 | 65373178 | 65373918 | chr9 | 135425263 | 135426339 |
| chr13 | 112229689 | 112230405 | chr20 | 29876987 | 29877594 | chr22 | 26133972 | 26136328 |
| chr10 | 125342725 | 125344898 | chr5 | 3767766 | 3768068 | chr1 | 24914371 | 24915029 |
| chr11 | 119363009 | 119363607 | chr19 | 42390286 | 42391514 | chr12 | 116694416 | 116695106 |
| chr12 | 50930558 | 50932853 | chr4 | 183364281 | 183365338 | chr5 | 178587800 | 178589236 |
| chr10 | 132143592 | 132144213 | chr21 | 43993126 | 43993509 | chr17 | 57497007 | 57497569 |
| chr6 | 168562620 | 168564151 | chr20 | 2820397 | 2820814 | chr10 | 127845012 | 127846020 |
| chr9 | 133127586 | 133127972 | chr17 | 50168396 | 50168969 | chr11 | 68215202 | 68215613 |
| chr12 | 50842851 | 50843223 | chr20 | 2535611 | 2537008 | chr22 | 30441060 | 30441794 |
| chr16 | 86930568 | 86931743 | chr11 | 45660917 | 45662436 | chr4 | 1508550 | 1509459 |
| chr7 | 150370314 | 150371722 | chr12 | 5081189 | 5081619 | chr7 | 54697930 | 54699297 |
| chr16 | 88404158 | 88404821 | chr9 | 88969201 | 88970056 | chr14 | 96625431 | 96626638 |
| chr12 | 117835631 | 117837156 | chr14 | 99675307 | 99676251 | chr11 | 126851417 | 126852353 |
| chr17 | 5592781 | 5593637 | chr16 | 26873184 | 26874772 | chr12 | 67442458 | 67443095 |
| chr13 | 113723388 | 113724273 | chr12 | 34393236 | 34393866 | chr2 | 235864526 | 235865577 |
| chr20 | 51802110 | 51802736 | chr21 | 10658895 | 10659480 | chr8 | 9269632 | 9270254 |
| chr20 | 64056840 | 64057365 | chr9 | 127566807 | 127567725 | chr21 | 8761124 | 8762208 |
| chr19 | 50461634 | 50462250 | chr19 | 13864237 | 13864730 | chr3 | 54891998 | 54894318 |
| chr5 | 79069875 | 79070583 | chr19 | 7993291 | 7994475 | chr9 | 135283452 | 135283888 |
| chr12 | 132176536 | 132177500 | chr11 | 2166941 | 2167850 | chr4 | 178415815 | 178416560 |
| chr8 | 26010497 | 26010767 | chr1 | 169460408 | 169461663 | chr13 | 48317680 | 48318339 |
| chr10 | 45198940 | 45199641 | chr14 | 105200751 | 105201181 | chr11 | 3453138 | 3454397 |
| chr8 | 142509425 | 142509760 | chr17 | 41492492 | 41493822 | chr2 | 235327556 | 235328132 |
| chr6 | 168280181 | 168281054 | chr15 | 89439375 | 89440150 | chr9 | 91577673 | 91578501 |
| chr19 | 51271107 | 51271639 | chr6 | 31791535 | 31793829 | chr18 | 8657948 | 8658505 |
| chr20 | 2748975 | 2750103 | chr6 | 34531442 | 34532294 | chr3 | 128603357 | 128604882 |
| chr18 | 76395572 | 76395928 | chr10 | 79376138 | 79376963 | chr3 | 128453428 | 128453832 |
| chr7 | 29566575 | 29566961 | chr22 | 41916905 | 41917751 | chr16 | 85484289 | 85485090 |
| chr20 | 6766301 | 6766963 | chr6 | 56999832 | 57000544 | chr6 | 167668004 | 167668520 |
| chr3 | 72152888 | 72153321 | chr11 | 93864981 | 93868222 | chr6 | 28724915 | 28728005 |
| chr2 | 120348983 | 120350528 | chr2 | 120739299 | 120741237 | chr5 | 153698896 | 153699107 |
| chr19 | 39247009 | 39247504 | chr2 | 102704576 | 102705193 | chr16 | 25907362 | 25908191 |
| chr11 | 114155389 | 114156460 | chr16 | 11592757 | 11594185 | chr10 | 126881904 | 126882308 |
| chr12 | 131216853 | 131217371 | chr9 | 136441426 | 136441984 | chr19 | 48501217 | 48501519 |
| chr3 | 159839926 | 159840435 | chr18 | 79052906 | 79053485 | chr11 | 115144689 | 115145626 |
| chr1 | 17149102 | 17149750 | chr16 | 1428036 | 1428279 | chr2 | 44972832 | 44973977 |
| chr7 | 39353923 | 39354299 | chr2 | 91411416 | 91411726 | chr16 | 2194182 | 2194699 |
| chr16 | 88170339 | 88171104 | chr3 | 112128943 | 112131472 | chr13 | 52906188 | 52906740 |
| chr20 | 56928697 | 56931672 | chr8 | 87933117 | 87935334 | chr17 | 9776895 | 9777385 |
| chr2 | 88284010 | 88284507 | chr5 | 1888185 | 1888968 | chr3 | 134769502 | 134773348 |
| chr5 | 181164786 | 181165029 | chr19 | 48265131 | 48265961 | chr6 | 160213623 | 160214868 |
| chr16 | 55283898 | 55284846 | chr12 | 34356616 | 34356864 | chr11 | 113457125 | 113458904 |
| chr12 | 5571148 | 5571473 | chr12 | 118470790 | 118471982 | chr22 | 48266077 | 48267241 |
| chr9 | 127978650 | 127979443 | chr2 | 154699431 | 154700278 | chr17 | 31568290 | 31568637 |
| chr12 | 131207455 | 131208119 | chr13 | 27962453 | 27963758 | chr20 | 44434160 | 44434677 |
| chr1 | 35573276 | 35574163 | chr13 | 99084760 | 99085454 | chr19 | 32047860 | 32048645 |
| chr16 | 13946829 | 13947250 | chr20 | 37912019 | 37912481 | chr7 | 157612402 | 157612815 |
| chr1 | 23436588 | 23437266 | chr4 | 173281716 | 173282665 | chr2 | 120465958 | 120466383 |
| chr14 | 105153224 | 105153758 | chr12 | 111202180 | 111203007 | chr2 | 138781244 | 138781612 |
| chr3 | 122184758 | 122184956 | chr2 | 10046653 | 10047471 | chr6 | 168346167 | 168346396 |
| chr9 | 136677252 | 136677541 | chr2 | 56719878 | 56720971 | chr20 | 45487371 | 45487686 |
| chr1 | 91535612 | 91535877 | chr22 | 50266978 | 50268846 | chr1 | 14944900 | 14945586 |
| chr7 | 143003092 | 143004446 | chr2 | 236117886 | 236118319 | chr11 | 1655229 | 1655759 |
| chr9 | 114721276 | 114722007 | chr14 | 103358890 | 103359921 | chr19 | 4015015 | 4015650 |
| chr16 | 67674764 | 67675900 | chr7 | 27177423 | 27177925 | chr3 | 127349729 | 127350121 |
| chr13 | 41453088 | 41456817 | chr12 | 3544379 | 3544909 | chr3 | 194218446 | 194219240 |
| chr7 | 95551919 | 95552419 | chr10 | 130476294 | 130476978 | chr3 | 24496097 | 24496946 |
| chr2 | 237563740 | 237564399 | chr1 | 106081072 | 106081358 | chr11 | 9759240 | 9759866 |
| chr10 | 8058984 | 8059604 | chr2 | 120553075 | 120553474 | chr5 | 171335652 | 171336546 |
| chr5 | 154690580 | 154691767 | chr16 | 85238165 | 85238528 | chr1 | 7534610 | 7535187 |
| chr4 | 3703837 | 3704447 | chr16 | 3011484 | 3012568 | chr12 | 128713516 | 128714442 |
| chr14 | 20434908 | 20435470 | chr12 | 38156427 | 38156703 | chr20 | 32513142 | 32513811 |
| chr15 | 93043477 | 93044076 | chr5 | 67350135 | 67351163 | chr1 | 230332505 | 230333065 |
| chr6 | 156507518 | 156508041 | chr12 | 130579133 | 130579846 | chr19 | 18786930 | 18787373 |
| chr10 | 133565006 | 133565671 | chr20 | 33122695 | 33124613 | chr15 | 22240607 | 22240909 |
| chr16 | 85806595 | 85806918 | chr14 | 100905801 | 100906909 | chr8 | 720813 | 722853 |
| chr5 | 492681 | 493499 | chr9 | 64013730 | 64014936 | chr13 | 60581571 | 60581989 |
| chr4 | 183721166 | 183722007 | chr19 | 55444957 | 55445525 | chr11 | 65803664 | 65804291 |
| chr9 | 97236152 | 97237671 | chr1 | 112969110 | 112969339 | chr1 | 6240409 | 6241216 |
| chr6 | 161890847 | 161891866 | chr17 | 49164135 | 49165164 | chr2 | 98901776 | 98902179 |
| chr4 | 49510164 | 49511356 | chr9 | 132486626 | 132487071 | chr1 | 6052544 | 6053490 |
| chr9 | 96156599 | 96157484 | chr15 | 92395501 | 92395826 | chr1 | 214106990 | 214107728 |
| chr10 | 1739565 | 1740010 | chr1 | 181179343 | 181181046 | chr6 | 26299029 | 26299743 |
| chr2 | 217916446 | 217917569 | chr15 | 26095831 | 26096928 | chr17 | 74053308 | 74053879 |
| chr7 | 2985064 | 2986066 | chr2 | 96927913 | 96928566 | chr22 | 39647870 | 39648316 |
| chr1 | 3858108 | 3858762 | chr17 | 21339073 | 21339744 | chr1 | 2469240 | 2469700 |
| chr11 | 44956712 | 44957479 | chr16 | 3364927 | 3365110 | chr8 | 128205399 | 128207382 |
| chr14 | 104566078 | 104567281 | chr10 | 122728091 | 122729187 | chr1 | 234111561 | 234112287 |
| chr16 | 27338756 | 27339415 | chr16 | 27067606 | 27068397 | chr13 | 27981194 | 27982378 |
| chr21 | 32834344 | 32835558 | chr8 | 2083902 | 2084952 | chr10 | 120962823 | 120963458 |
| chr6 | 165671596 | 165674548 | chr8 | 87868828 | 87869743 | chr11 | 67699878 | 67700259 |
| chr9 | 33748609 | 33749165 | chr16 | 84042840 | 84043532 | chr17 | 77255407 | 77255911 |
| chr12 | 71731850 | 71732324 | chr4 | 824166 | 824482 | chr9 | 32631853 | 32633685 |
| chr9 | 79691447 | 79694201 | chr10 | 84237087 | 84237487 | chr8 | 141559724 | 141560859 |
| chr8 | 133852359 | 133854547 | chr9 | 36159923 | 36160358 | chr16 | 2035074 | 2036002 |
| chr20 | 63036442 | 63037096 | chr12 | 54158688 | 54159465 | chr15 | 93037306 | 93037943 |
| chr3 | 44221863 | 44222987 | chr22 | 27288187 | 27289104 | chr10 | 133246628 | 133247431 |
| chr6 | 17588287 | 17588916 | chr5 | 17061945 | 17062420 | chr17 | 79485412 | 79486185 |
| chr19 | 15021196 | 15021817 | chr6 | 163750871 | 163751729 | chr9 | 4083904 | 4085722 |
| chr1 | 1285896 | 1286275 | chr9 | 95074481 | 95075046 | chr15 | 64748215 | 64749043 |
| chr1 | 229396605 | 229397055 | chr2 | 20212478 | 20213003 | chr22 | 50640300 | 50641038 |
| chr18 | 11146693 | 11147666 | chr6 | 1146956 | 1147399 | chr4 | 7391236 | 7391713 |
| chr7 | 130492949 | 130493292 | chr2 | 218033472 | 218034240 | chr19 | 2371165 | 2371425 |
| chr15 | 77732623 | 77733160 | chr19 | 50794089 | 50795152 | chr17 | 77830253 | 77831069 |
| chr6 | 44228224 | 44229462 | chr6 | 167237936 | 167238658 | chr14 | 101684722 | 101685338 |
| chr5 | 139642066 | 139643145 | chr4 | 7340428 | 7340858 | chr8 | 26034891 | 26038606 |
| chr22 | 22379718 | 22380610 | chr8 | 41645086 | 41645777 | chr11 | 119701868 | 119702321 |
| chr4 | 39432972 | 39433839 | chr8 | 54174483 | 54174837 | chr16 | 84482476 | 84482840 |
| chr16 | 2253601 | 2254239 | chr12 | 111324122 | 111324982 | chr10 | 90915587 | 90917369 |
| chr8 | 965818 | 966107 | chr8 | 30971189 | 30974376 | chr5 | 3321779 | 3323021 |
| chr9 | 32955683 | 32955993 | chr12 | 111774768 | 111775268 | chr1 | 2926402 | 2927639 |
| chr22 | 41950761 | 41951831 | chr8 | 37460436 | 37460774 | chr10 | 116743564 | 116744154 |
| chr1 | 155125981 | 155126591 | chr10 | 132193221 | 132193844 | chr19 | 38482351 | 38482801 |
| chr22 | 17795722 | 17796846 | chr12 | 131831484 | 131832944 | chr17 | 74130501 | 74131208 |
| chr11 | 114989140 | 114990632 | chr13 | 22793602 | 22794254 | chr1 | 202694448 | 202696390 |
| chr1 | 30379115 | 30379768 | chr15 | 93071406 | 93072009 | chr4 | 7570204 | 7571678 |
| chr10 | 132133022 | 132133631 | chr6 | 71587728 | 71588722 | chr10 | 128463185 | 128464896 |
| chr16 | 57797925 | 57798508 | chr13 | 31019878 | 31020784 | chr17 | 73691299 | 73692175 |
| chr1 | 230710504 | 230711316 | chr12 | 120545790 | 120548505 | chr12 | 108514086 | 108514321 |
| chr19 | 45028685 | 45029441 | chr13 | 113749460 | 113750031 | chr9 | 137863321 | 137864303 |
| chr9 | 93681135 | 93682180 | chr8 | 124236241 | 124237175 | chr19 | 41025729 | 41026171 |
| chr9 | 134809726 | 134810390 | chr12 | 3920529 | 3920989 | chr16 | 70656643 | 70657168 |
| chr18 | 74290723 | 74291173 | chr22 | 22791610 | 22792922 | chr5 | 171441146 | 171441659 |
| chr1 | 18877468 | 18877782 | chr10 | 128430438 | 128431241 | chr21 | 44764919 | 44765766 |
| chr19 | 46654709 | 46655278 | chr9 | 32819913 | 32822057 | chr11 | 132346911 | 132347725 |
| chr5 | 151696278 | 151696895 | chr15 | 40843001 | 40843649 | chr8 | 46410363 | 46410962 |
| chr3 | 196967268 | 196967986 | chr15 | 29451297 | 29451722 | chr5 | 179398208 | 179400158 |
| chr22 | 24506241 | 24508473 | chr20 | 64164454 | 64165334 | chr19 | 3831250 | 3831729 |
| chr19 | 43465170 | 43466120 | chr2 | 129993070 | 129993607 | chr6 | 158663190 | 158664067 |
| chr22 | 25112241 | 25112773 | chr10 | 117142246 | 117143683 | chr4 | 7620147 | 7620811 |
| chr8 | 142004329 | 142005044 | chr16 | 34956713 | 34957243 | chr2 | 9692586 | 9693007 |
| chr18 | 12121426 | 12123141 | chr16 | 30027544 | 30028735 | chr20 | 53942214 | 53944434 |
| chr20 | 63481498 | 63482270 | chr2 | 218831617 | 218831973 | chr5 | 73370223 | 73372039 |
| chr17 | 2903953 | 2904393 | chr5 | 6224734 | 6226156 | chr19 | 40489334 | 40489815 |
| chr17 | 52160020 | 52160513 | chr8 | 16981882 | 16982459 | chr10 | 129206813 | 129207711 |
| chr11 | 115462281 | 115462529 | chr1 | 2868154 | 2868759 | chr7 | 148400172 | 148400778 |
| chr10 | 30186280 | 30186952 | chr12 | 121898744 | 121899379 | chr22 | 26244303 | 26245589 |
| chr12 | 34378847 | 34379730 | chr20 | 57593343 | 57594073 | chr14 | 16097875 | 16098411 |
| chr2 | 238120481 | 238121075 | chr17 | 35479186 | 35480265 | chr10 | 662888 | 663588 |
| chr2 | 24827441 | 24828106 | chr13 | 112342653 | 112343064 | chr11 | 133156192 | 133157561 |
| chr4 | 3743827 | 3744839 | chr11 | 125425646 | 125426496 | chr9 | 126616304 | 126617033 |
| chr11 | 62921473 | 62922193 | chr1 | 2978361 | 2979319 | chr19 | 11423062 | 11424074 |
| chr15 | 80452018 | 80453044 | chr6 | 30597413 | 30599628 | chr2 | 216000025 | 216001502 |
| chr22 | 35548754 | 35549593 | chr10 | 131743905 | 131744420 | chr7 | 4791962 | 4793082 |
| chr2 | 74501697 | 74502105 | chr5 | 179529582 | 179530131 | chr18 | 11564838 | 11565964 |
| chr3 | 44058385 | 44059139 | chr10 | 8504454 | 8505286 | chr5 | 16756957 | 16758211 |
| chr11 | 125243421 | 125244752 | chr2 | 1796095 | 1796601 | chr22 | 26290878 | 26292542 |
| chr9 | 76362127 | 76362979 | chr16 | 21685002 | 21685806 | chr5 | 2197153 | 2197708 |
| chr19 | 1494212 | 1494786 | chr16 | 12544208 | 12544765 | chr8 | 8702719 | 8703178 |
| chr21 | 38727691 | 38728795 | chr3 | 125824692 | 125826797 | chr17 | 74117118 | 74118470 |
| chr7 | 100799804 | 100800460 | chr8 | 143809328 | 143809923 | chr20 | 2754865 | 2756238 |
| chr17 | 259404 | 260153 | chr20 | 61136104 | 61136664 | chr2 | 236053273 | 236054297 |
| chr20 | 52106375 | 52106743 | chr2 | 200696312 | 200697914 | chr22 | 22355687 | 22357186 |
| chr4 | 52860878 | 52861812 | chr16 | 35504862 | 35506150 | chr9 | 114118154 | 114118840 |
| chr19 | 30373053 | 30373996 | chr2 | 218882189 | 218882844 | chr5 | 135035184 | 135035745 |
| chr8 | 142075865 | 142076294 | chr13 | 108494627 | 108495049 | chr20 | 38944161 | 38944812 |
| chr19 | 53787400 | 53788037 | chr16 | 9297398 | 9297734 | chr22 | 36797633 | 36798013 |
| chr9 | 135086485 | 135087013 | chr19 | 48195594 | 48196918 | chr3 | 43858367 | 43859440 |
| chr9 | 111600073 | 111600415 | chr20 | 46640577 | 46641043 | chr17 | 79046503 | 79046982 |
| chr13 | 40172163 | 40172475 | chr16 | 46464809 | 46465322 | chr8 | 21782486 | 21782836 |
| chr18 | 78506093 | 78506452 | chr20 | 62523908 | 62524799 | chr10 | 131636110 | 131636790 |
| chr10 | 15694960 | 15696462 | chr17 | 35665272 | 35668459 | chr20 | 60069708 | 60071113 |
| chr1 | 211511440 | 211513814 | chr19 | 31715423 | 31715847 | chr19 | 31061815 | 31063222 |
| chr5 | 167155203 | 167155913 | chr8 | 46123223 | 46124645 | chr1 | 5522704 | 5523088 |
| chr10 | 34905877 | 34906631 | chr12 | 123555881 | 123556505 | chr20 | 61507929 | 61508349 |
| chr9 | 37412261 | 37412960 | chr10 | 101290180 | 101290826 | chr14 | 74951621 | 74952127 |
| chr6 | 150008678 | 150009905 | chr14 | 95714045 | 95714741 | chr8 | 46422100 | 46422643 |
| chr1 | 2132247 | 2132839 | chr10 | 1705237 | 1706185 | chr2 | 94984953 | 94985704 |
| chr2 | 126970755 | 126971644 | chr20 | 37520202 | 37521482 | chr5 | 176626569 | 176627368 |
| chr18 | 74457984 | 74458497 | chr17 | 73590088 | 73590441 | chr7 | 62302323 | 62303923 |
| chr4 | 189096184 | 189097036 | chr10 | 99523002 | 99523296 | chr5 | 141419093 | 141419775 |
| chr16 | 86295872 | 86296858 | chr13 | 114048024 | 114048687 | chr17 | 5225846 | 5227096 |
| chr20 | 25859713 | 25860745 | chr6 | 170030819 | 170031455 | chr1 | 230392211 | 230392783 |
| chr6 | 42910884 | 42911497 | chr18 | 76953421 | 76955368 | chr9 | 134215506 | 134216099 |
| chr7 | 44321197 | 44321814 | chr5 | 598080 | 598922 | chr22 | 42769611 | 42770465 |
| chr3 | 141094765 | 141095292 | chr12 | 58176413 | 58180444 | chr17 | 79702236 | 79702695 |
| chr8 | 142518737 | 142519027 | chr1 | 38244180 | 38244577 | chr17 | 82704780 | 82705191 |
| chr1 | 1758449 | 1759197 | chr20 | 64047902 | 64048742 | chr15 | 77630559 | 77631322 |
| chr5 | 1911778 | 1912580 | chr8 | 141679695 | 141680344 | chr3 | 126341537 | 126342107 |
| chr10 | 1932846 | 1933443 | chr13 | 67225879 | 67227963 | chr19 | 50192899 | 50193512 |
| chr14 | 97458658 | 97459377 | chr13 | 111845368 | 111845968 | chr19 | 45346437 | 45346796 |
| chr18 | 75390944 | 75391815 | chr5 | 116770704 | 116772056 | chr21 | 41368971 | 41370367 |
| chr11 | 8226570 | 8227106 | chr17 | 45255804 | 45256747 | chr8 | 141947776 | 141948409 |
| chr2 | 219636595 | 219637076 | chr6 | 30112412 | 30115294 | chr2 | 71893608 | 71894002 |
| chr14 | 99563254 | 99563692 | chr8 | 14457275 | 14457972 | chr2 | 226180446 | 226181303 |
| chr8 | 69832174 | 69832473 | chr11 | 126337156 | 126337987 | chr9 | 130437172 | 130437695 |
| chr19 | 17821100 | 17821551 | chr8 | 11612293 | 11613001 | chr10 | 121484578 | 121485078 |
| chr16 | 85569567 | 85570012 | chr8 | 22161557 | 22162801 | chr11 | 61904253 | 61904795 |
| chr20 | 760599 | 761231 | chr1 | 246793078 | 246795426 | chr20 | 61882958 | 61883638 |
| chr9 | 135030670 | 135032294 | chr5 | 1656906 | 1657535 | chr1 | 208076782 | 208077932 |
| chr2 | 221766345 | 221769349 | chr22 | 17035438 | 17036231 | chr9 | 124029706 | 124030271 |
| chr4 | 187505556 | 187505780 | chr8 | 141507717 | 141508728 | chr2 | 1514592 | 1515202 |
| chr18 | 76588631 | 76589198 | chr13 | 112050994 | 112052541 | chr16 | 25925485 | 25926282 |
| chr3 | 123799838 | 123800070 | chr16 | 23707396 | 23708504 | chr20 | 48687080 | 48687962 |
| chr10 | 47599842 | 47600363 | chr2 | 89840024 | 89840951 | chr6 | 1014368 | 1016432 |
| chr12 | 110594 | 111930 | chr3 | 194368622 | 194369052 | chr4 | 7956340 | 7957333 |
| chr1 | 14600920 | 14602945 | chr16 | 28537323 | 28538366 | chr9 | 72478642 | 72479587 |
| chr16 | 69107734 | 69108555 | chr8 | 142470091 | 142470596 | chr2 | 121018617 | 121019040 |
| chr1 | 70706498 | 70707851 | chr9 | 29779218 | 29780087 | chr1 | 210342990 | 210344016 |
| chr8 | 628087 | 629307 | chr9 | 135291246 | 135292084 | chr12 | 34361848 | 34363000 |
| chr22 | 20423651 | 20424381 | chr19 | 40718252 | 40719209 | chr16 | 7814969 | 7816920 |
| chr10 | 1309911 | 1310780 | chr16 | 1096135 | 1096553 | chr19 | 39127607 | 39128404 |
| chr17 | 76524837 | 76525478 | chr12 | 108303132 | 108304031 | chr22 | 44595105 | 44595587 |
| chr14 | 90454241 | 90454538 | chr2 | 6130137 | 6133283 | chr4 | 6493233 | 6494184 |
| chr16 | 54430417 | 54431549 | chr10 | 23342940 | 23343655 | chr16 | 84284155 | 84284890 |
| chr20 | 56690433 | 56691183 | chr9 | 124289957 | 124290487 | chr1 | 30217856 | 30219159 |
| chr5 | 154219023 | 154219686 | chr5 | 173864075 | 173865530 | chr18 | 27921535 | 27922051 |
| chr15 | 93358147 | 93359966 | chr2 | 239832738 | 239833236 | chr17 | 42548565 | 42549213 |
| chr4 | 189790243 | 189791348 | chr11 | 8031570 | 8032159 | chr2 | 5756302 | 5757761 |
| chr6 | 30371543 | 30373359 | chr16 | 1023662 | 1024665 | chr1 | 240525769 | 240526453 |
| chr1 | 219929223 | 219929742 | chr9 | 130596336 | 130597799 | chr16 | 85378260 | 85379067 |
| chr10 | 277320 | 277829 | chr10 | 1809186 | 1809487 | chr16 | 27110290 | 27110881 |
| chr16 | 2858628 | 2859199 | chr19 | 14231326 | 14232359 | chr11 | 133081319 | 133081966 |
| chr4 | 49161213 | 49161646 | chr21 | 32432920 | 32433489 | chr8 | 22045599 | 22046279 |
| chr21 | 10122861 | 10123304 | chr20 | 64192304 | 64193392 | chr20 | 36350942 | 36351405 |
| chr6 | 110356796 | 110357401 | chr14 | 100657479 | 100657990 | chr21 | 13847046 | 13849115 |
| chr1 | 8210139 | 8210691 | chr15 | 73757979 | 73759061 | chr12 | 79565240 | 79566093 |
| chr2 | 219433951 | 219434862 | chr7 | 135164670 | 135165148 | chr15 | 29825674 | 29827528 |
| chr19 | 16719292 | 16720202 | chr19 | 56718657 | 56719920 | chr1 | 935492 | 935836 |
| chr12 | 94560663 | 94561053 | chr22 | 34958170 | 34960235 | chr2 | 120483116 | 120483568 |
| chr10 | 38526659 | 38527365 | chr1 | 968947 | 970841 | chr9 | 132168582 | 132169324 |
| chr6 | 10466213 | 10466696 | chr16 | 86284166 | 86285982 | chr16 | 34766252 | 34767058 |
| chr8 | 41447422 | 41448163 | chr1 | 46474106 | 46474822 | chr10 | 30641167 | 30641934 |
| chr1 | 180953567 | 180954907 | chr17 | 33280085 | 33281843 | chr17 | 10328826 | 10329709 |
| chr8 | 53883311 | 53883818 | chr12 | 130710176 | 130711023 | chr19 | 55710028 | 55711609 |
| chr17 | 1907448 | 1907859 | chr11 | 70522910 | 70523250 | chr11 | 16603669 | 16604034 |
| chr5 | 956179 | 957159 | chr10 | 132164843 | 132165238 | chr5 | 101782760 | 101784196 |
| chr22 | 36565997 | 36566649 | chr12 | 21773355 | 21773573 | chr1 | 245128579 | 245129231 |
| chr16 | 35140797 | 35141308 | chr5 | 1153424 | 1153783 | chr12 | 128550803 | 128551588 |
| chr12 | 131624470 | 131625220 | chr1 | 5451776 | 5452573 | chr12 | 3107027 | 3107318 |
| chr19 | 2511398 | 2511764 | chr19 | 50518584 | 50518995 | chr3 | 32592950 | 32593234 |
| chr11 | 67650090 | 67650743 | chr18 | 73715231 | 73715753 | chr1 | 18725610 | 18726839 |
| chr10 | 1598936 | 1599783 | chr19 | 52493644 | 52494212 | chr11 | 79253035 | 79253688 |
| chr1 | 41487216 | 41487726 | chr19 | 12982283 | 12985027 | chr5 | 1709532 | 1710086 |
| chr2 | 98312023 | 98312534 | chr10 | 103668525 | 103669025 | chr19 | 41844583 | 41844912 |
| chr2 | 240213250 | 240213849 | chr19 | 40231461 | 40231953 | chr5 | 5844300 | 5847909 |
| chr19 | 23003043 | 23003413 | chr15 | 40280126 | 40280594 | chr17 | 36675328 | 36676689 |
| chr2 | 237922627 | 237923502 | chr20 | 40684842 | 40686572 | chr1 | 92480895 | 92482148 |
| chr6 | 122842107 | 122842406 | chr3 | 10327764 | 10328883 | chr2 | 119116998 | 119117972 |
| chr2 | 106579023 | 106581217 | chr5 | 150289803 | 150290251 | chr5 | 176571778 | 176572704 |
| chr12 | 4959950 | 4960404 | chr8 | 142391423 | 142391900 | chr9 | 131822994 | 131823996 |
| chr6 | 41599543 | 41600689 | chr22 | 37418828 | 37419498 | chr10 | 1715940 | 1717374 |
| chr12 | 131101663 | 131102321 | chr20 | 56735268 | 56736015 | chr1 | 239996601 | 239997580 |
| chr5 | 8870191 | 8871111 | chr7 | 434962 | 435518 | chr3 | 127467339 | 127467831 |
| chr7 | 130350934 | 130351483 | chr11 | 9464312 | 9466277 | chr10 | 131514256 | 131514758 |
| chr12 | 112854889 | 112856379 | chr8 | 144376634 | 144377136 | chr1 | 1928402 | 1928758 |
| chr17 | 49989899 | 49990837 | chr1 | 143260582 | 143261215 | chr14 | 104872919 | 104873635 |
| chr11 | 64637936 | 64638580 | chr17 | 83124910 | 83125393 | chr4 | 3305176 | 3306032 |
| chr10 | 129784972 | 129785558 | chr14 | 92251864 | 92252584 | chr3 | 115791951 | 115793178 |
| chr18 | 9968621 | 9969139 | chr9 | 129571398 | 129572025 | chr4 | 164186139 | 164188269 |
| chr2 | 120373525 | 120374198 | chr10 | 11157050 | 11158319 | chr20 | 41354413 | 41354783 |
| chr1 | 234379519 | 234380956 | chr10 | 96292787 | 96295261 | chr2 | 44906910 | 44907324 |
| chr8 | 12952762 | 12953123 | chr1 | 229229080 | 229229923 | chr2 | 233164648 | 233166757 |
| chr10 | 124670993 | 124672020 | chr9 | 133602796 | 133603647 | chr15 | 74300188 | 74300662 |
| chr8 | 11345579 | 11346554 | chr2 | 47572214 | 47572909 | chr12 | 111635819 | 111636554 |
| chr7 | 100825889 | 100826539 | chr20 | 61185931 | 61186538 | chr7 | 2450521 | 2451260 |
| chr17 | 79921956 | 79922537 | chr6 | 170123380 | 170123707 | chr15 | 74209039 | 74210075 |
| chr2 | 101464840 | 101465903 | chr4 | 54149477 | 54149867 | chr16 | 9815526 | 9817937 |
| chr18 | 21896892 | 21897424 | chr8 | 8704185 | 8705384 | chr17 | 73785273 | 73786051 |
| chr5 | 2007958 | 2008403 | chr10 | 62813459 | 62813994 | chr1 | 30807275 | 30807665 |
| chr10 | 7532783 | 7533190 | chr1 | 2112169 | 2112537 | chr18 | 31410871 | 31411359 |
| chr9 | 135614188 | 135614623 | chr14 | 60347542 | 60349399 | chr1 | 228754766 | 228755107 |
| chr11 | 78463957 | 78464248 | chr6 | 163940885 | 163943201 | chr12 | 6773150 | 6773647 |
| chr9 | 134373922 | 134375276 | chr1 | 16534973 | 16535233 | chr2 | 229576792 | 229578432 |
| chr21 | 41727418 | 41728420 | chr5 | 152142061 | 152144386 | chr7 | 3722377 | 3722770 |
| chr2 | 8543725 | 8544092 | chr3 | 75666297 | 75667970 | chr20 | 46053569 | 46055019 |
| chr5 | 139664986 | 139665931 | chr21 | 10705014 | 10705637 | chr14 | 95236849 | 95237536 |
| chr7 | 70878178 | 70879537 | chr15 | 31329575 | 31329855 | chr8 | 2893910 | 2895260 |
| chr6 | 170136832 | 170137792 | chr1 | 1065285 | 1065796 | chr11 | 10729150 | 10729765 |
| chr21 | 43282632 | 43283071 | chr2 | 120660493 | 120661822 | chr17 | 81741389 | 81742163 |
| chr17 | 42783889 | 42784803 | chr16 | 57985724 | 57986450 | chr11 | 128361604 | 128362980 |
| chr1 | 148264875 | 148265330 | chr12 | 255417 | 255918 | chr16 | 3180555 | 3181706 |
| chr11 | 370260 | 370673 | chr22 | 47148555 | 47149377 | chr18 | 7459884 | 7460306 |
| chr9 | 130446049 | 130446720 | chr8 | 2767443 | 2770671 | chr5 | 2570593 | 2571001 |
| chr10 | 132457352 | 132458082 | chr10 | 127103146 | 127103708 | chr5 | 181084735 | 181085523 |
| chr22 | 45246444 | 45247407 | chr3 | 68377623 | 68378739 | chr7 | 155360267 | 155360701 |
| chr2 | 131040179 | 131040629 | chr12 | 116269022 | 116270536 | chr2 | 628719 | 629290 |
| chr1 | 3351252 | 3352759 | chr10 | 122115492 | 122117684 | chr4 | 76255935 | 76256487 |
| chr4 | 167233037 | 167233525 | chr8 | 142600878 | 142601606 | chr16 | 88995056 | 88995611 |
| chr20 | 20277025 | 20277650 | chr3 | 186845663 | 186845979 | chr17 | 27113305 | 27114377 |
| chr6 | 1618607 | 1619265 | chr13 | 35976242 | 35978367 | chr5 | 2607254 | 2608396 |
| chr16 | 86291683 | 86292691 | chr20 | 62889002 | 62889280 | chr18 | 10240626 | 10242312 |
| chr5 | 149264594 | 149265338 | chr8 | 10965628 | 10965919 | chr1 | 16693657 | 16693756 |
| chr18 | 26439771 | 26440979 | chr3 | 72462721 | 72463465 | chr17 | 76080596 | 76081312 |
| chr22 | 26900776 | 26901552 | chr4 | 6196938 | 6197348 | chr14 | 34297877 | 34300678 |
| chr9 | 126497482 | 126498217 | chr20 | 63470144 | 63470975 | chr16 | 57722574 | 57723135 |
| chr11 | 2271576 | 2272080 | chr19 | 50685956 | 50686943 | chr2 | 2535642 | 2536935 |
| chr2 | 232367910 | 232368454 | chr15 | 81134060 | 81134360 | chr9 | 122225953 | 122227714 |
| chr18 | 47594617 | 47596301 | chr19 | 8932349 | 8932816 | chr19 | 29286119 | 29288059 |
| chr17 | 8116594 | 8117335 | chr19 | 43651963 | 43652796 | chr20 | 36339865 | 36340618 |
| chr1 | 209353967 | 209354311 | chr20 | 51508213 | 51508552 | chr19 | 52451237 | 52451932 |
| chr2 | 105177810 | 105178680 | chr12 | 17016873 | 17018237 | chr20 | 60585993 | 60587396 |
| chr8 | 55371294 | 55374018 | chr5 | 1133494 | 1134299 | chr6 | 6255484 | 6258896 |
| chr17 | 63477962 | 63478392 | chr17 | 81733058 | 81733610 | chr1 | 245130761 | 245131346 |
| chr6 | 53405510 | 53406167 | chr2 | 218855366 | 218856066 | chr22 | 49026304 | 49027170 |
| chr4 | 83534797 | 83535074 | chr18 | 79458301 | 79458627 | chr6 | 161243214 | 161243509 |
| chr12 | 95546025 | 95547883 | chr5 | 135134045 | 135134540 | chr8 | 69356592 | 69357245 |
| chr15 | 98951538 | 98951896 | chr22 | 45440740 | 45441153 | chr1 | 18408791 | 18409368 |
| chr19 | 50460088 | 50460847 | chr12 | 49293326 | 49294659 | chr15 | 45099810 | 45100137 |
| chr19 | 34669099 | 34669573 | chr2 | 161239183 | 161239736 | chr12 | 124546161 | 124546858 |
| chr19 | 54416468 | 54418305 | chr15 | 26688211 | 26689499 | chr7 | 150980613 | 150983441 |
| chr10 | 13439366 | 13439926 | chr2 | 25167408 | 25167892 | chr2 | 174025516 | 174025843 |
| chr19 | 55957264 | 55958979 | chr12 | 129783519 | 129785170 | chr16 | 66867159 | 66870123 |
| chr19 | 54350394 | 54352455 | chr10 | 43942652 | 43943980 | chr5 | 174335867 | 174336324 |
| chr11 | 118869871 | 118870755 | chr2 | 36380903 | 36381736 | chr11 | 66294627 | 66295131 |
| chr2 | 72994720 | 72995665 | chr20 | 62357832 | 62358668 | chr1 | 53594016 | 53594409 |
| chr1 | 24920478 | 24921178 | chr20 | 54064097 | 54065017 | chr17 | 82444529 | 82445016 |
| chr1 | 155299726 | 155300623 | chr1 | 150024334 | 150025220 | chr16 | 34750286 | 34750881 |
| chr2 | 1949093 | 1949822 | chr16 | 86759034 | 86760014 | chr17 | 72561057 | 72561516 |
| chr12 | 109772967 | 109773711 | chr8 | 57586213 | 57587807 | chr3 | 71432244 | 71433998 |
| chr4 | 8937728 | 8938443 | chr15 | 31223908 | 31224674 | chr1 | 220278035 | 220280599 |
| chr1 | 7251009 | 7251235 | chr8 | 25384126 | 25386245 | chr7 | 113955762 | 113958120 |
| chr8 | 46255696 | 46256993 | chr13 | 87675487 | 87677182 | chr3 | 10764100 | 10764998 |
| chr17 | 81713041 | 81714178 | chr14 | 36161663 | 36162387 | chr3 | 133783696 | 133784032 |
| chr7 | 95324341 | 95325144 | chr16 | 4537038 | 4537792 | chr9 | 37038958 | 37039436 |
| chr20 | 62117216 | 62117899 | chr5 | 41509251 | 41509811 | chr8 | 48923571 | 48924054 |
| chr2 | 240965674 | 240966433 | chr8 | 73954576 | 73955466 | chr9 | 126677701 | 126678234 |
| chr8 | 22047371 | 22048076 | chr8 | 10891653 | 10892987 | chr1 | 200033488 | 200033999 |
| chr14 | 94164992 | 94165604 | chr10 | 20767100 | 20768507 | chr17 | 27476143 | 27477682 |
| chr6 | 68639296 | 68640239 | chr1 | 214673005 | 214673674 | chr1 | 2646508 | 2648469 |
| chr22 | 41831075 | 41831555 | chr14 | 97025821 | 97026222 | chr5 | 31930420 | 31931347 |
| chr22 | 46078510 | 46078900 | chr22 | 39114326 | 39114856 | chr21 | 8758930 | 8759142 |
| chr2 | 240914241 | 240914492 | chr9 | 138116976 | 138117861 | chr21 | 39601027 | 39602220 |
| chr16 | 79438254 | 79439160 | chr17 | 16585859 | 16587168 | chr1 | 163957757 | 163958395 |
| chr5 | 132655465 | 132655921 | chr2 | 66648022 | 66648448 | chr20 | 63477561 | 63478080 |
| chr2 | 15981027 | 15981680 | chr15 | 24877806 | 24878308 | chr5 | 169063165 | 169063807 |
| chr2 | 129878285 | 129878651 | chr18 | 7010907 | 7011465 | chr11 | 134430921 | 134434151 |
| chr17 | 74115198 | 74116063 | chr3 | 125990055 | 125991384 | chr7 | 60912288 | 60912906 |
| chr8 | 143181217 | 143181663 | chr10 | 133446001 | 133446647 | chr22 | 39592303 | 39592747 |
| chr5 | 3326165 | 3326302 | chr9 | 134345036 | 134346068 | chr2 | 236176894 | 236177454 |
| chr2 | 131753621 | 131755624 | chr9 | 131812900 | 131813536 | chr15 | 93372496 | 93373164 |
| chr10 | 129852856 | 129853668 | chr5 | 2520450 | 2521628 | chr17 | 3280909 | 3281939 |
| chr9 | 135413427 | 135413911 | chr9 | 134282901 | 134283597 | chr22 | 49232489 | 49232895 |
| chr9 | 132425969 | 132427572 | chr4 | 1569028 | 1569336 | chr10 | 70459778 | 70460461 |
| chr3 | 45230997 | 45233014 | chr1 | 227786239 | 227787003 | chr18 | 14357172 | 14357809 |
| chr19 | 42095743 | 42096508 | chr22 | 44332610 | 44333259 | chr9 | 130327742 | 130327991 |
| chr10 | 44910558 | 44911555 | chr5 | 3962173 | 3962595 | chr19 | 1740628 | 1741206 |
| chr22 | 38088723 | 38089709 | chr6 | 163867363 | 163867903 | chr8 | 10569041 | 10570046 |
| chr16 | 3097266 | 3099012 | chr19 | 34832818 | 34833038 | chr8 | 107756162 | 107756867 |
| chr2 | 121828525 | 121828733 | chr2 | 20675877 | 20677062 | chr1 | 36913690 | 36915029 |
| chr17 | 79950723 | 79951418 | chr17 | 79562074 | 79562716 | chr7 | 62343121 | 62344691 |
| chr2 | 232691374 | 232693198 | chr17 | 50291967 | 50293952 | chr10 | 117804275 | 117804926 |
| chr19 | 11624166 | 11625791 | chr17 | 28368417 | 28369065 | chr1 | 109273742 | 109275243 |
| chr15 | 99005442 | 99006078 | chr19 | 11674332 | 11674804 | chr10 | 31697337 | 31698438 |
| chr1 | 17603476 | 17604525 | chr19 | 55928071 | 55931292 | chr6 | 36333951 | 36334594 |
| chr12 | 106144841 | 106147618 | chr8 | 11465415 | 11465942 | chr4 | 3792137 | 3792701 |
| chr18 | 70499666 | 70500017 | chr16 | 89161925 | 89162182 | chr20 | 63002955 | 63003541 |
| chr13 | 111972416 | 111973876 | chr11 | 2698802 | 2699234 | chr4 | 7141277 | 7142745 |
| chr22 | 45335735 | 45336023 | chr19 | 48568599 | 48569069 | chr8 | 143344656 | 143345187 |
| chr1 | 155285655 | 155286052 | chr4 | 185013918 | 185014543 | chr19 | 22805190 | 22806604 |
| chr12 | 117032698 | 117033638 | chr17 | 73813697 | 73814508 | chr7 | 100642282 | 100642925 |
| chr1 | 2654588 | 2655743 | chr14 | 104785740 | 104786218 | chr12 | 130030664 | 130031648 |
| chr9 | 129557443 | 129558142 | chr6 | 29511421 | 29514077 | chr16 | 34712989 | 34713726 |
| chr6 | 49713437 | 49713959 | chr8 | 46417020 | 46417578 | chr12 | 583481 | 584268 |
| chr2 | 2564121 | 2564990 | chr8 | 41754597 | 41755225 | chr20 | 46291231 | 46291672 |
| chr5 | 2183236 | 2183949 | chr19 | 2358888 | 2359405 | chr12 | 50080978 | 50081435 |
| chr1 | 29530777 | 29532729 | chr5 | 176793406 | 176794090 | chr15 | 70268637 | 70269139 |
| chr10 | 125162346 | 125162838 | chr8 | 10997447 | 10999795 | chr1 | 18156778 | 18157494 |
| chr5 | 95854309 | 95855600 | chr1 | 3471808 | 3472181 | chr12 | 126782958 | 126784550 |
| chr8 | 142544522 | 142544764 | chr9 | 121990487 | 121991481 | chr14 | 94451442 | 94455213 |
| chr7 | 157130362 | 157134019 | chr19 | 45780088 | 45780898 | chr16 | 52780712 | 52781836 |
| chr21 | 46161199 | 46161587 | chr17 | 49890543 | 49891271 | chr17 | 79396121 | 79396898 |
| chr10 | 112224845 | 112226703 | chr2 | 8538626 | 8539374 | chr1 | 2231244 | 2232033 |
| chr7 | 44064389 | 44065468 | chr18 | 78720904 | 78721104 | chr7 | 140435669 | 140435912 |
| chr11 | 61747588 | 61749650 | chr22 | 49671842 | 49673291 | chr16 | 89101882 | 89102109 |
| chr22 | 40714703 | 40715386 | chr19 | 30447239 | 30448262 | chr2 | 131672038 | 131672515 |
| chr2 | 224482959 | 224485613 | chr17 | 30235004 | 30235472 | chr4 | 55079809 | 55080788 |
| chr1 | 156675771 | 156676319 | chr8 | 52343219 | 52343672 | chr10 | 18260747 | 18261306 |
| chr10 | 100065205 | 100065560 | chr16 | 34698652 | 34700780 | chr9 | 138049971 | 138050686 |
| chr16 | 804235 | 805170 | chr19 | 41696800 | 41697764 | chr12 | 132509689 | 132510072 |
| chr16 | 2962546 | 2963076 | chr11 | 71205988 | 71206720 | chr12 | 3279482 | 3280598 |
| chr20 | 21020529 | 21021486 | chr10 | 9378606 | 9379519 | chr7 | 142336784 | 142337261 |
| chr7 | 56092640 | 56093428 | chr16 | 46499230 | 46500268 | chr10 | 98590208 | 98593443 |
| chr7 | 149732103 | 149732716 | chr2 | 83879196 | 83880161 | chr4 | 587891 | 588417 |
| chr18 | 30838398 | 30838844 | chr7 | 157767749 | 157768540 | chr13 | 87810356 | 87810946 |
| chr17 | 14303835 | 14304547 | chr10 | 120194989 | 120198322 | chr7 | 61069596 | 61070021 |
| chr7 | 1742079 | 1742670 | chr1 | 156844427 | 156844869 | chr10 | 75276160 | 75276862 |
| chr15 | 97875369 | 97876222 | chr9 | 132805098 | 132805693 | chr16 | 86806964 | 86807509 |
| chr10 | 42169822 | 42170697 | chr11 | 46913083 | 46913468 | chr17 | 76957696 | 76958480 |
| chr14 | 44962912 | 44964527 | chr19 | 35524677 | 35525208 | chr9 | 133916016 | 133916458 |
| chr7 | 71752596 | 71755697 | chr14 | 97417711 | 97419314 | chr20 | 57168280 | 57168935 |
| chr7 | 5370515 | 5371193 | chr4 | 104189944 | 104190481 | chr1 | 175505327 | 175505567 |
| chr7 | 1697970 | 1698902 | chr2 | 42522959 | 42523597 | chr22 | 45071693 | 45072516 |
| chr16 | 86573813 | 86575062 | chr16 | 87555465 | 87555970 | chr17 | 48627933 | 48628664 |
| chr11 | 711040 | 711722 | chr7 | 2728895 | 2729174 | chr1 | 18099889 | 18100396 |
| chr12 | 130729477 | 130729902 | chr5 | 141239879 | 141242736 | chr12 | 117258054 | 117258583 |
| chr10 | 128686044 | 128686353 | chr22 | 37325474 | 37327269 | chr3 | 51707245 | 51707644 |
| chr5 | 181070294 | 181070946 | chr17 | 37770661 | 37771420 | chr7 | 3892412 | 3892983 |
| chr1 | 202021395 | 202022180 | chr1 | 54354319 | 54354824 | chr1 | 228003499 | 228004318 |
| chr9 | 135782484 | 135783169 | chr17 | 343190 | 343710 | chr1 | 5718739 | 5719394 |
| chr5 | 2415523 | 2416200 | chr19 | 17239917 | 17240640 | chr22 | 49193373 | 49193713 |
| chr13 | 24637685 | 24638190 | chr2 | 129935328 | 129937821 | chr1 | 232747699 | 232749337 |
| chr4 | 8784955 | 8785876 | chr9 | 97307273 | 97308528 | chr18 | 14179366 | 14179944 |
| chr15 | 26547886 | 26549808 | chr4 | 4673133 | 4673342 | chr9 | 137880589 | 137881147 |
| chr14 | 23388492 | 23388898 | chr13 | 106690469 | 106691480 | chr10 | 123388301 | 123390454 |
| chr14 | 104219453 | 104220375 | chr22 | 16783720 | 16785100 | chr20 | 62590742 | 62591219 |
| chr12 | 130455110 | 130455595 | chr1 | 232178603 | 232181104 | chr11 | 133778320 | 133778741 |
| chr1 | 223771843 | 223773013 | chr8 | 7157314 | 7158922 | chr15 | 93109721 | 93110313 |
| chr10 | 129021625 | 129021956 | chr3 | 194296610 | 194297035 | chr22 | 47389262 | 47390133 |
| chr4 | 25707809 | 25708341 | chr22 | 31110864 | 31112881 | chr3 | 170440911 | 170442616 |
| chr22 | 27202753 | 27203244 | chr22 | 19971134 | 19971614 | chr16 | 34158771 | 34159166 |
| chr8 | 142608435 | 142608773 | chr12 | 52881625 | 52882332 | chr4 | 3662161 | 3662441 |
| chr20 | 63294984 | 63296023 | chr20 | 64142417 | 64143454 | chr1 | 24896870 | 24897469 |
| chr8 | 52480718 | 52481385 | chr11 | 129386190 | 129387845 | chr11 | 134923537 | 134924316 |
| chr16 | 75206345 | 75206651 | chr12 | 119205782 | 119209034 | chr11 | 73344105 | 73344608 |
| chr2 | 172109900 | 172110412 | chr16 | 5411427 | 5412725 | chr19 | 16136260 | 16136873 |
| chr4 | 173499637 | 173500375 | chr4 | 8657247 | 8657748 | chr6 | 31135299 | 31135855 |
| chr11 | 2158189 | 2159565 | chr19 | 50546877 | 50547498 | chr1 | 220888473 | 220888918 |
| chr16 | 1120094 | 1120618 | chr11 | 9094385 | 9094938 | chr20 | 63343402 | 63344412 |
| chr13 | 112134403 | 112134924 | chr7 | 98657732 | 98658349 | chr16 | 85220374 | 85220721 |
| chr12 | 130680514 | 130680870 | chr3 | 8650733 | 8651643 | chr14 | 67503043 | 67503829 |
| chr4 | 151674883 | 151675286 | chr17 | 21693120 | 21693637 | chr15 | 94579315 | 94580499 |
| chr9 | 125436829 | 125437346 | chr8 | 142198299 | 142198798 | chr6 | 150341624 | 150342065 |
| chr7 | 43839319 | 43839593 | chr16 | 88468906 | 88469925 | chr4 | 189721539 | 189722788 |
| chr19 | 30076013 | 30076505 | chr3 | 190273561 | 190273834 | chr6 | 88486105 | 88487115 |
| chr7 | 27253089 | 27253397 | chr20 | 46199213 | 46199739 | chr20 | 44015358 | 44016185 |
| chr17 | 5077127 | 5078031 | chr11 | 43546790 | 43547623 | chr7 | 52302036 | 52302595 |
| chr5 | 525112 | 526377 | chr9 | 62833511 | 62834035 | chr2 | 232350999 | 232352173 |
| chr14 | 104460185 | 104461014 | chr7 | 60937185 | 60937448 | chr11 | 128748493 | 128749562 |
| chr5 | 179569573 | 179572322 | chr1 | 229408126 | 229409189 | chr20 | 37464047 | 37464474 |
| chr19 | 7857980 | 7858424 | chr19 | 6513707 | 6514688 | chr18 | 78309669 | 78311111 |
| chr10 | 129939555 | 129939958 | chr9 | 89676354 | 89676609 | chr9 | 133638459 | 133639173 |
| chr21 | 42335939 | 42336482 | chr19 | 5561879 | 5563788 | chr21 | 36033662 | 36034760 |
| chr1 | 234586300 | 234587428 | chr2 | 218890632 | 218891784 | chr7 | 151011222 | 151011994 |
| chr5 | 71807506 | 71810299 | chr11 | 111976851 | 111976977 | chr14 | 36615775 | 36618221 |
| chr1 | 119003623 | 119004906 | chr4 | 27090376 | 27091203 | chr19 | 13009982 | 13010888 |
| chr11 | 116612796 | 116613652 | chr6 | 30885896 | 30886775 | chr16 | 78939644 | 78941452 |
| chr7 | 62297556 | 62298726 | chr19 | 12751323 | 12752349 | chr3 | 57162361 | 57163465 |
| chr7 | 154371099 | 154372282 | chr19 | 46480705 | 46482054 | chr10 | 33050489 | 33051254 |
| chr21 | 44485318 | 44486685 | chr6 | 39924250 | 39925206 | chr8 | 71840062 | 71841640 |
| chr7 | 140517390 | 140517919 | chr14 | 23143951 | 23145355 | chr8 | 6382269 | 6384302 |
| chr16 | 1390222 | 1390687 | chr15 | 77704675 | 77705704 | chr22 | 29479113 | 29479685 |
| chr7 | 93043413 | 93043677 | chr10 | 117554485 | 117555384 | chr17 | 77362716 | 77363215 |
| chr1 | 31589162 | 31589808 | chr16 | 86337013 | 86337757 | chr5 | 415030 | 416043 |
| chr18 | 78984958 | 78985645 | chr8 | 56117791 | 56118583 | chr19 | 7696671 | 7696898 |
| chr9 | 131503766 | 131504399 | chr17 | 77047210 | 77047689 | chr4 | 6414996 | 6415835 |
| chr22 | 50315921 | 50316413 | chr8 | 10964653 | 10965179 | chr8 | 489775 | 491465 |
| chr14 | 62371891 | 62373109 | chr12 | 132387190 | 132387457 | chr10 | 49442202 | 49442592 |
| chr4 | 1492888 | 1493522 | chr12 | 120668789 | 120669678 | chr5 | 1100792 | 1101086 |
| chr1 | 10669145 | 10669844 | chr8 | 37273848 | 37275171 | chr12 | 4975650 | 4976024 |
| chr14 | 57068536 | 57069965 | chr1 | 41394657 | 41396396 | chr1 | 232363331 | 232363911 |
| chr3 | 183595287 | 183596439 | chr3 | 128496276 | 128497151 | chr20 | 58512493 | 58513720 |
| chr10 | 117781341 | 117782480 | chr8 | 143435784 | 143436283 | chr4 | 7324429 | 7324750 |
| chr18 | 58847014 | 58847435 | chr20 | 46153115 | 46153644 | chr7 | 156608085 | 156608398 |
| chr11 | 64033967 | 64034918 | chr9 | 131834924 | 131835604 | chr1 | 26201371 | 26201968 |
| chr2 | 169715604 | 169716355 | chr4 | 94450182 | 94450786 | chr4 | 7700158 | 7700808 |
| chr2 | 130251880 | 130253434 | chr16 | 5386377 | 5387696 | chr19 | 50213317 | 50215440 |
| chr19 | 15900424 | 15900944 | chr11 | 2190783 | 2191096 | chr17 | 82121697 | 82122630 |
| chr20 | 63547441 | 63548764 | chr12 | 34307311 | 34307938 | chr2 | 237350481 | 237351355 |
| chr5 | 173314838 | 173315750 | chr4 | 184402076 | 184402808 | chr18 | 77252114 | 77252352 |
| chr8 | 43292022 | 43292347 | chr1 | 34457983 | 34459882 | chr6 | 43926476 | 43926903 |
| chr9 | 122336953 | 122337577 | chr1 | 11700644 | 11701198 | chr10 | 52778583 | 52779123 |
| chr2 | 238107626 | 238108272 | chr5 | 139383575 | 139385172 | chr2 | 176763349 | 176765279 |
| chr9 | 128392818 | 128393787 | chr19 | 50348678 | 50349806 | chr13 | 25700778 | 25703300 |
| chr4 | 182139226 | 182140678 | chr8 | 98972400 | 98973195 | chr20 | 57136265 | 57136819 |
| chr14 | 104383598 | 104384151 | chr22 | 19748733 | 19749511 | chr14 | 104293853 | 104294347 |
| chr15 | 26891979 | 26892767 | chr17 | 33970731 | 33971387 | chr19 | 447993 | 448396 |
| chr7 | 1843597 | 1844204 | chr20 | 57586278 | 57587103 | chr1 | 18060777 | 18061586 |
| chr20 | 48384199 | 48385056 | chr18 | 11870157 | 11872397 | chr3 | 135575011 | 135577208 |
| chr20 | 57707144 | 57707976 | chr12 | 127007128 | 127009240 | chr8 | 40039732 | 40040334 |
| chr10 | 101073100 | 101074968 | chr10 | 42951312 | 42952293 | chr7 | 139583625 | 139584499 |
| chr9 | 131792605 | 131793034 | chr14 | 96593156 | 96593613 | chr12 | 114417483 | 114419425 |
| chr22 | 45632636 | 45633733 | chr16 | 27805860 | 27808287 | chr20 | 2694498 | 2694898 |
| chr10 | 103927517 | 103928489 | chr7 | 155397357 | 155397684 | chr19 | 50720309 | 50720726 |
| chr12 | 132356723 | 132356958 | chr14 | 94762761 | 94764261 | chr4 | 7207404 | 7208012 |
| chr6 | 41239596 | 41239878 | chr15 | 23897520 | 23898736 | chr11 | 3154347 | 3155326 |
| chr18 | 78795521 | 78796564 | chr17 | 79049626 | 79050104 | chr1 | 157398960 | 157402391 |
| chr16 | 89623462 | 89624214 | chr9 | 132166625 | 132167119 | chr1 | 1069791 | 1070054 |
| chr15 | 40068903 | 40069689 | chr12 | 34371047 | 34371971 | chr9 | 127865134 | 127865698 |
| chr10 | 9634151 | 9635635 | chr8 | 32480166 | 32483428 | chr2 | 29369975 | 29371411 |
| chr17 | 4588081 | 4588601 | chr1 | 13575904 | 13576563 | chr9 | 121581647 | 121582185 |
| chr19 | 751330 | 752936 | chr1 | 33551157 | 33554724 | chr9 | 6122996 | 6125955 |
| chr10 | 34657781 | 34658906 | chr22 | 48099564 | 48099937 | chr17 | 50564938 | 50565734 |
| chr2 | 86301064 | 86303144 | chr2 | 218966906 | 218967513 | chr10 | 97016965 | 97017259 |
| chr9 | 137387650 | 137388341 | chr11 | 34251235 | 34252287 | chr14 | 97976907 | 97978101 |
| chr20 | 3671727 | 3672265 | chr22 | 35126679 | 35127284 | chr5 | 3275291 | 3277203 |
| chr2 | 25204540 | 25205283 | chr8 | 923029 | 923606 | chr5 | 179637641 | 179640993 |
| chr5 | 6773105 | 6773683 | chr7 | 6551144 | 6551890 | chr22 | 37819441 | 37820131 |
| chr9 | 87834010 | 87834818 | chr21 | 40322611 | 40323879 | chr19 | 44306329 | 44306773 |
| chr6 | 36961848 | 36962359 | chr12 | 54217812 | 54218254 | chr10 | 16519558 | 16519881 |
| chr12 | 56510950 | 56511146 | chr16 | 1548206 | 1549000 | chr8 | 72928439 | 72929569 |
| chr17 | 82270644 | 82271232 | chr19 | 30253448 | 30254260 | chr14 | 104477970 | 104478582 |
| chr17 | 35752446 | 35753393 | chr5 | 34747399 | 34750208 | chr5 | 684089 | 684828 |
| chr6 | 167824418 | 167824910 | chr1 | 1067476 | 1067660 | chr20 | 17783981 | 17784897 |
| chr6 | 170225873 | 170226257 | chr4 | 183987358 | 183987866 | chr1 | 34265580 | 34266720 |
| chr10 | 42241110 | 42242816 | chr2 | 26693616 | 26694512 | chr8 | 747366 | 748302 |
| chr2 | 68979828 | 68982497 | chr12 | 131556023 | 131556420 | chr6 | 27215462 | 27215991 |
| chr16 | 4011369 | 4011855 | chr17 | 34499734 | 34503056 | chr19 | 13502529 | 13504105 |
| chr2 | 2705205 | 2706188 | chr16 | 87457060 | 87457992 | chr7 | 154914290 | 154914806 |
| chr16 | 86714635 | 86715819 | chr22 | 27311663 | 27313536 | chr10 | 3015970 | 3017134 |
| chr15 | 94702210 | 94703103 | chr17 | 34400947 | 34404248 | chr14 | 90491382 | 90492003 |
| chr12 | 34304926 | 34305505 | chr22 | 22211466 | 22212155 | chr12 | 122024015 | 122024388 |
| chr15 | 27587106 | 27589228 | chr8 | 28633490 | 28634447 | chr12 | 130482076 | 130482447 |
| chr10 | 83917561 | 83917815 | chr14 | 78714407 | 78714932 | chr11 | 116733984 | 116734715 |
| chr17 | 204780 | 205469 | chr7 | 70614352 | 70615053 | chr9 | 135411145 | 135411637 |
| chr6 | 168089700 | 168089947 | chr16 | 2469392 | 2470192 | chr11 | 68651926 | 68652215 |
| chr10 | 46050651 | 46051742 | chr16 | 3013583 | 3014066 | chr10 | 116879352 | 116880871 |
| chr6 | 39901804 | 39902200 | chr1 | 11546484 | 11548324 | chr20 | 29763784 | 29764634 |
| chr9 | 121510532 | 121511588 | chr2 | 113272660 | 113274950 | chr2 | 154586409 | 154586750 |
| chr4 | 190040957 | 190041579 | chr18 | 37362477 | 37364319 | chr17 | 10033861 | 10034600 |
| chr21 | 38713920 | 38715584 | chr18 | 77989568 | 77990474 | chr7 | 27247066 | 27248796 |
| chr5 | 56480104 | 56480593 | chr16 | 85396239 | 85396911 | chr1 | 27360843 | 27361205 |
| chr6 | 37511842 | 37512655 | chr8 | 46133577 | 46134120 | chr10 | 126224568 | 126225188 |
| chr16 | 1018957 | 1019760 | chr17 | 40673668 | 40675124 | chr2 | 67895466 | 67896430 |
| chr9 | 131837362 | 131838986 | chr12 | 98790326 | 98791492 | chr10 | 114548005 | 114549379 |
| chr14 | 100823841 | 100824669 | chr2 | 38465887 | 38467042 | chr16 | 54992737 | 54995452 |
| chr2 | 238439062 | 238439549 | chr14 | 89025847 | 89026521 | chr15 | 96285778 | 96287187 |
| chr8 | 46176663 | 46177673 | chr2 | 2711025 | 2712079 | chr2 | 21043452 | 21044480 |
| chr13 | 111541872 | 111542212 | chr14 | 94988335 | 94991299 | chr15 | 74280096 | 74280734 |
| chr21 | 10336760 | 10337021 | chr7 | 155137758 | 155139676 | chr21 | 10596668 | 10597771 |
| chr17 | 3985211 | 3986422 | chr12 | 107795211 | 107796411 | chr1 | 111896037 | 111896247 |
| chr22 | 21714382 | 21714752 | chr4 | 3577029 | 3578605 | chr2 | 94587105 | 94587796 |
| chr12 | 34337669 | 34338492 | chr7 | 284799 | 285191 | chr7 | 27207656 | 27208438 |
| chr6 | 33634510 | 33635515 | chr8 | 16978839 | 16979579 | chr10 | 93363620 | 93364003 |
| chr18 | 79819251 | 79820089 | chr8 | 56508222 | 56509506 | chr21 | 42777923 | 42778344 |
| chr1 | 205434925 | 205436342 | chr13 | 28590122 | 28591089 | chr20 | 62051041 | 62051555 |
| chr3 | 38115284 | 38116557 | chr11 | 961979 | 962665 | chr7 | 100011754 | 100012607 |
| chr16 | 34736008 | 34736584 | chr16 | 22763780 | 22765897 | chr12 | 130678877 | 130679591 |
| chr17 | 41316033 | 41316376 | chr16 | 36134385 | 36135591 | chr6 | 166904131 | 166904460 |
| chr4 | 6657852 | 6658424 | chr11 | 134047509 | 134048225 | chr15 | 99446946 | 99447726 |
| chr7 | 27179167 | 27179794 | chr16 | 86298077 | 86298907 | chr11 | 44520264 | 44520549 |
| chr17 | 40014159 | 40015116 | chr3 | 48594817 | 48595154 | chr22 | 46129672 | 46130335 |
| chr22 | 46672611 | 46673270 | chr1 | 1128464 | 1128743 | chr4 | 7578583 | 7579839 |
| chr17 | 14298211 | 14298954 | chr3 | 16091129 | 16093104 | chr7 | 67588896 | 67589954 |
| chr7 | 158515311 | 158516035 | chr7 | 1369990 | 1370940 | chr7 | 157477997 | 157478236 |
| chr14 | 74953917 | 74954835 | chr20 | 62585537 | 62585877 | chr9 | 135604586 | 135605382 |
| chr12 | 113756376 | 113757950 | chr16 | 477232 | 478111 | chr1 | 2269442 | 2270474 |
| chr1 | 34647532 | 34650645 | chr1 | 85628711 | 85629434 | chr19 | 1734154 | 1734524 |
| chr16 | 86730653 | 86731137 | chr9 | 135353227 | 135353731 | chr8 | 11013861 | 11014436 |
| chr18 | 58784623 | 58785262 | chr3 | 197001861 | 197002267 | chr22 | 43412954 | 43413469 |
| chr16 | 57528450 | 57529507 | chr12 | 131618357 | 131618843 | chr8 | 3332356 | 3334220 |
| chr1 | 226882461 | 226883825 | chr8 | 6256462 | 6257810 | chr19 | 43682987 | 43683857 |
| chr6 | 27653074 | 27653365 | chr1 | 2535245 | 2535865 | chr10 | 117838607 | 117838952 |
| chr1 | 11355720 | 11356209 | chr6 | 35509297 | 35510340 | chr15 | 101615854 | 101616489 |
| chr5 | 135547771 | 135549597 | chr5 | 150521069 | 150521654 | chr14 | 104282661 | 104283207 |
| chr12 | 42551718 | 42553300 | chr22 | 43178511 | 43178724 | chr14 | 20216361 | 20216528 |
| chr7 | 128879705 | 128880140 | chr11 | 3476224 | 3477780 | chr16 | 86824897 | 86825584 |
| chr15 | 50514463 | 50515342 | chr19 | 49091050 | 49091328 | chr13 | 29503110 | 29503374 |
| chr17 | 797287 | 798004 | chr10 | 13904140 | 13904505 | chr15 | 88491050 | 88491511 |
| chr17 | 16744932 | 16746456 | chr20 | 59756291 | 59756670 | chr22 | 49202952 | 49203536 |
| chr22 | 17857259 | 17857511 | chr12 | 116505998 | 116506676 | chr3 | 57013806 | 57014114 |
| chr5 | 125200443 | 125201335 | chr18 | 59705971 | 59707538 | chr10 | 117684930 | 117685256 |
| chr6 | 41636742 | 41637002 | chr6 | 168132070 | 168133078 | chr17 | 76123842 | 76124707 |
| chr3 | 45758987 | 45759265 | chr7 | 159126041 | 159126863 | chr19 | 4267372 | 4269299 |
| chr10 | 26970767 | 26972837 | chr22 | 39500939 | 39501749 | chr20 | 61352372 | 61353131 |
| chr19 | 35812511 | 35813109 | chr1 | 207424504 | 207425044 | chr8 | 23321466 | 23322276 |
| chr7 | 101722323 | 101722641 | chr2 | 62229068 | 62230548 | chr16 | 68727968 | 68728515 |
| chr7 | 155385354 | 155385740 | chr16 | 2798585 | 2799143 | chr12 | 54658709 | 54659652 |
| chr1 | 35049427 | 35049627 | chr15 | 26184991 | 26187477 | chr5 | 4514109 | 4515729 |
| chr9 | 134358757 | 134359470 | chr12 | 5718453 | 5719261 | chr2 | 218989726 | 218990347 |
| chr20 | 63151853 | 63152447 | chr2 | 230268863 | 230270437 | chr8 | 57455173 | 57456870 |
| chr19 | 53189834 | 53191078 | chr6 | 420731 | 423357 | chr10 | 3066139 | 3066554 |
| chr19 | 46270585 | 46271168 | chr7 | 405831 | 406364 | chr14 | 103280183 | 103280751 |
| chr10 | 71975636 | 71976083 | chr9 | 135339779 | 135340229 | chr12 | 47788066 | 47788348 |
| chr9 | 93039152 | 93040478 | chr12 | 33437682 | 33438330 | chr1 | 202180373 | 202180982 |
| chr20 | 57270529 | 57271477 | chr1 | 1062703 | 1063301 | chr13 | 31055023 | 31056533 |
| chr16 | 89155399 | 89156379 | chr1 | 2806388 | 2807163 | chr10 | 132188244 | 132188736 |
| chr5 | 1831796 | 1832828 | chr17 | 47783556 | 47786591 | chr12 | 41187383 | 41187974 |
| chr2 | 153455429 | 153455818 | chr13 | 28057558 | 28058035 | chr16 | 49012694 | 49013684 |
| chr12 | 130686152 | 130686899 | chr12 | 125276489 | 125276992 | chr16 | 1176032 | 1176929 |
| chr6 | 28643468 | 28645121 | chr16 | 34078534 | 34079482 | chr11 | 70912400 | 70912950 |
| chr9 | 19030620 | 19032622 | chr7 | 101103033 | 101104348 | chr3 | 53169392 | 53171478 |
| chr7 | 44374126 | 44376050 | chr13 | 106087413 | 106089542 | chr3 | 10499155 | 10499677 |
| chr16 | 1088154 | 1088585 | chr8 | 46202026 | 46202673 | chr1 | 3880001 | 3880863 |
| chr20 | 47352083 | 47352972 | chr9 | 121701150 | 121701466 | chr7 | 73833275 | 73833703 |
| chr11 | 2132804 | 2133777 | chr4 | 7851573 | 7852882 | chr9 | 137667090 | 137667554 |
| chr20 | 59747101 | 59747388 | chr10 | 95230706 | 95231440 | chr2 | 240799582 | 240800522 |
| chr19 | 7815492 | 7815971 | chr1 | 18675516 | 18676057 | chr18 | 24727405 | 24728005 |
| chr19 | 7797068 | 7799130 | chr12 | 34309847 | 34310149 | chr1 | 246725372 | 246727513 |
| chr1 | 52928220 | 52928665 | chr1 | 230476582 | 230477015 | chr12 | 130120218 | 130120878 |
| chr9 | 14804352 | 14805127 | chr11 | 75669235 | 75670027 | chr1 | 10735170 | 10735788 |
| chr14 | 23717909 | 23720530 | chr11 | 13315396 | 13316473 | chr2 | 91571261 | 91571649 |
| chr15 | 29149768 | 29150055 | chr8 | 133169164 | 133171480 | chr3 | 11306963 | 11308826 |
| chr16 | 49697924 | 49698532 | chr2 | 20688431 | 20691267 | chr17 | 58520694 | 58521642 |
| chr19 | 5484577 | 5485079 | chr6 | 5087213 | 5087853 | chr10 | 43221552 | 43222016 |
| chr9 | 136147983 | 136148607 | chr5 | 2387810 | 2388387 | chr13 | 112217663 | 112218677 |
| chr6 | 27268688 | 27270003 | chr21 | 38989495 | 38990165 | chr1 | 156103808 | 156105564 |
| chr6 | 170271977 | 170272380 | chr10 | 127495978 | 127496459 | chr20 | 49452083 | 49452794 |
| chr20 | 45337132 | 45337929 | chr17 | 78558093 | 78558747 | chr16 | 88561108 | 88561674 |
| chr11 | 49049911 | 49050212 | chr15 | 77727863 | 77728579 | chr16 | 35492364 | 35492974 |
| chr1 | 205481826 | 205482083 | chr12 | 108240262 | 108240785 | chr19 | 47002465 | 47003520 |
| chr17 | 8873780 | 8874968 | chr1 | 153418390 | 153421417 | chr9 | 128015683 | 128016358 |
| chr8 | 46245855 | 46247083 | chr21 | 45012245 | 45012555 | chr10 | 111078647 | 111079450 |
| chr6 | 36849051 | 36849427 | chr12 | 130639687 | 130640435 | chr17 | 34541761 | 34544120 |
| chr3 | 19233701 | 19234187 | chr10 | 79120324 | 79121388 | chr17 | 14697621 | 14699425 |
| chr12 | 52951790 | 52952468 | chr5 | 181099151 | 181100370 | chr7 | 73665603 | 73666907 |
| chr2 | 239482992 | 239484327 | chr4 | 3784633 | 3785094 | chr5 | 1228087 | 1228652 |
| chr20 | 2574765 | 2576599 | chr17 | 74150268 | 74150943 | chr13 | 112079356 | 112080156 |
| chr22 | 33650246 | 33650611 | chr1 | 25572119 | 25573374 | chr7 | 155214450 | 155214845 |
| chr5 | 38846714 | 38847588 | chr11 | 71610908 | 71611877 | chr5 | 170202056 | 170203023 |
| chr20 | 62979100 | 62979679 | chr1 | 29524384 | 29524979 | chr12 | 130503651 | 130504594 |
| chr19 | 38528091 | 38528749 | chr12 | 34332216 | 34332785 | chr17 | 9024597 | 9025134 |
| chr5 | 173290469 | 173291241 | chr12 | 102832831 | 102833072 | chr2 | 26620192 | 26620646 |
| chr20 | 56736840 | 56737788 | chr14 | 104304473 | 104304720 | chr16 | 56960810 | 56961883 |
| chr11 | 2838414 | 2839847 | chr19 | 546277 | 547147 | chr9 | 119632139 | 119634473 |
| chr14 | 92347142 | 92348471 | chr11 | 71306519 | 71307042 | chr16 | 35238241 | 35241228 |
| chr16 | 88090176 | 88090655 | chr12 | 125773188 | 125773645 | chr16 | 23509115 | 23509476 |
| chr17 | 79191430 | 79191931 | chr16 | 1932919 | 1933738 | chr6 | 39887526 | 39887953 |
| chr20 | 32514770 | 32515297 | chr8 | 142149158 | 142149597 | chr19 | 3369513 | 3371245 |
| chr18 | 47748486 | 47749821 | chr1 | 14725274 | 14726634 | chr20 | 48454386 | 48455210 |
| chr7 | 81610941 | 81611288 | chr17 | 41528953 | 41529853 | chr4 | 16351351 | 16352319 |
| chr6 | 71419398 | 71419969 | chr19 | 30691030 | 30691792 | chr16 | 46843920 | 46844434 |
| chr10 | 124545092 | 124545910 | chr6 | 1217957 | 1218625 | chr10 | 127145383 | 127145900 |
| chr8 | 592491 | 592876 | chr7 | 138147070 | 138147585 | chr16 | 26955110 | 26956892 |
| chr14 | 101364631 | 101365187 | chr11 | 12914350 | 12914886 | chr9 | 98174937 | 98176454 |
| chr21 | 43811268 | 43812178 | chr19 | 18287744 | 18288560 | chr3 | 44531568 | 44534413 |
| chr5 | 1137070 | 1137709 | chr15 | 93361647 | 93363155 | chr19 | 512497 | 513967 |
| chr12 | 54134993 | 54137364 | chr19 | 13574724 | 13575496 | chr2 | 74732042 | 74733570 |
| chr6 | 170179001 | 170179252 | chr14 | 102760460 | 102761318 | chr1 | 62324684 | 62325560 |
| chr16 | 83189160 | 83192150 | chr7 | 55213984 | 55214907 | chr16 | 86532211 | 86532544 |
| chr16 | 1262377 | 1262871 | chr16 | 9154499 | 9154816 | chr9 | 94325722 | 94325970 |
| chr19 | 1373530 | 1374383 | chr19 | 14271665 | 14272611 | chr8 | 142795237 | 142795859 |
| chr17 | 9946542 | 9946945 | chr5 | 150209463 | 150209870 | chr8 | 97867811 | 97868351 |
| chr16 | 87126595 | 87127489 | chr11 | 32400022 | 32400494 | chr1 | 37318799 | 37320211 |
| chr21 | 36571686 | 36572292 | chr14 | 98906077 | 98907142 | chr12 | 119165158 | 119166959 |
| chr22 | 27238980 | 27240198 | chr5 | 179258479 | 179259052 | chr9 | 135105907 | 135106254 |
| chr17 | 6454842 | 6455285 | chr1 | 177968866 | 177969914 | chr17 | 80471101 | 80471642 |
| chr7 | 38691539 | 38692180 | chr17 | 21765360 | 21766278 | chr17 | 45858435 | 45858978 |
| chr19 | 43405847 | 43406627 | chr16 | 5504774 | 5506959 | chr9 | 138072725 | 138073220 |
| chr3 | 132135750 | 132137842 | chr8 | 100458332 | 100459376 | chr11 | 1142693 | 1145218 |
| chr11 | 123578197 | 123578947 | chr16 | 50973106 | 50974852 | chr9 | 38746496 | 38747228 |
| chr12 | 114903011 | 114904526 | chr2 | 120227741 | 120228188 | chr15 | 87828377 | 87830422 |
| chr7 | 92641862 | 92645861 | chr9 | 35790928 | 35791152 | chr5 | 6434853 | 6435116 |
| chr16 | 6680475 | 6683311 | chr1 | 7672641 | 7673232 | chr12 | 132373638 | 132374096 |
| chr18 | 80199768 | 80200393 | chr10 | 129871179 | 129871780 | chr15 | 99961130 | 99961330 |
| chr20 | 58840253 | 58841365 | chr7 | 127254898 | 127255946 | chr19 | 35704223 | 35704482 |
| chr19 | 46282452 | 46282822 | chr10 | 132001011 | 132001894 | chr11 | 133809098 | 133810127 |
| chr12 | 3137073 | 3137973 | chr1 | 24915791 | 24916302 | chr16 | 25776171 | 25778872 |
| chr20 | 63949807 | 63950178 | chr17 | 78397063 | 78397966 | chr20 | 574077 | 574736 |
| chr10 | 132951645 | 132952279 | chr2 | 74883165 | 74885361 | chr20 | 40681098 | 40682293 |
| chr17 | 4467333 | 4468220 | chr22 | 49051790 | 49052538 | chr1 | 3804166 | 3804839 |
| chr17 | 34591530 | 34592705 | chr7 | 30731350 | 30731785 | chr2 | 28956602 | 28957337 |
| chr2 | 91844058 | 91844747 | chr17 | 45790233 | 45790806 | chr13 | 113607668 | 113608511 |
| chr6 | 161444447 | 161445299 | chr16 | 28059714 | 28060763 | chr2 | 239894882 | 239896409 |
| chr2 | 60273984 | 60274472 | chr1 | 3248428 | 3248899 | chr10 | 13194419 | 13195181 |
| chr7 | 879215 | 879547 | chr19 | 4293683 | 4294346 | chr7 | 158570410 | 158570878 |
| chr10 | 26092150 | 26092945 | chr14 | 102545461 | 102545898 | chr10 | 11869745 | 11870359 |
| chr3 | 157631318 | 157633410 | chr21 | 10667547 | 10668282 | chr3 | 72336587 | 72337789 |
| chr20 | 63028629 | 63030373 | chr1 | 146393497 | 146394406 | chr14 | 24418587 | 24419805 |
| chr20 | 57843916 | 57845896 | chr19 | 10586627 | 10587242 | chr11 | 18046350 | 18046574 |
| chr10 | 8296111 | 8296685 | chr6 | 21385276 | 21385580 | chr19 | 5894728 | 5895516 |
| chr12 | 34291081 | 34291717 | chr9 | 130451021 | 130451385 | chr12 | 54023475 | 54025136 |
| chr11 | 119371490 | 119372490 | chr3 | 32902548 | 32903102 | chr20 | 56219852 | 56222033 |
| chr3 | 130448710 | 130449660 | chr20 | 64188935 | 64189782 | chr19 | 33736212 | 33737151 |
| chr16 | 73702984 | 73703960 | chr16 | 84555504 | 84556095 | chr5 | 141364153 | 141364574 |
| chr11 | 112881361 | 112881761 | chr2 | 241901532 | 241902121 | chr9 | 95068885 | 95070179 |
| chr19 | 5074837 | 5075394 | chr7 | 1882026 | 1882634 | chr14 | 105169423 | 105170265 |
| chr9 | 134553773 | 134554587 | chr20 | 63073864 | 63074755 | chr8 | 37219261 | 37220398 |
| chr5 | 171520366 | 171520782 | chr20 | 49998756 | 49999578 | chr15 | 45471941 | 45472434 |
| chr1 | 116646263 | 116647217 | chr1 | 3688462 | 3689091 | chr11 | 134393672 | 134394477 |
| chr3 | 191499164 | 191500031 | chr2 | 74415756 | 74416215 | chr7 | 5272606 | 5273101 |
| chr13 | 111602850 | 111603682 | chr9 | 61670725 | 61671428 | chr7 | 155925240 | 155926017 |
| chr3 | 178536246 | 178537597 | chr3 | 125821270 | 125822510 | chr19 | 19547113 | 19548277 |
| chr18 | 9017082 | 9018105 | chr10 | 69962621 | 69963388 | chr5 | 132023916 | 132025171 |
| chr11 | 32970647 | 32971923 | chr1 | 4930891 | 4931729 | chr15 | 81117765 | 81118743 |
| chr6 | 122744270 | 122745634 | chr10 | 131147015 | 131147571 | chr16 | 34084651 | 34085765 |
| chr4 | 114063270 | 114064855 | chr1 | 169915270 | 169915710 | chr17 | 38840185 | 38841021 |
| chr16 | 86219796 | 86220305 | chr8 | 143707801 | 143708438 | chr1 | 2543699 | 2544523 |
| chr19 | 56054150 | 56055292 | chr5 | 2137031 | 2137287 | chr10 | 549468 | 550257 |
| chr8 | 42179537 | 42180231 | chr22 | 45919025 | 45919494 | chr19 | 48716731 | 48717229 |
| chr1 | 927091 | 928220 | chr14 | 77787236 | 77790474 | chr7 | 5382199 | 5383217 |
| chr1 | 30214283 | 30215521 | chr1 | 44996469 | 44996997 | chr1 | 17825650 | 17826092 |
| chr9 | 133396681 | 133397471 | chr7 | 150960438 | 150961736 | chr11 | 1198596 | 1199377 |
| chr20 | 53631515 | 53632756 | chr4 | 8959594 | 8960417 | chr1 | 1022910 | 1023552 |
| chr20 | 61896068 | 61896974 | chr20 | 1778535 | 1779145 | chr19 | 47469686 | 47470200 |
| chr16 | 34598008 | 34598711 | chr11 | 67615718 | 67617046 | chr15 | 73375436 | 73375880 |
| chr15 | 31367519 | 31368581 | chr19 | 48895082 | 48895608 | chr19 | 31319611 | 31320473 |
| chr11 | 103738949 | 103739392 | chr15 | 82648455 | 82649240 | chr22 | 47605154 | 47605504 |
| chr3 | 46576887 | 46578578 | chr11 | 123196565 | 123197802 | chr22 | 20308235 | 20308845 |
| chr20 | 51372889 | 51373312 | chr12 | 131573520 | 131574001 | chr5 | 141339675 | 141340358 |
| chr1 | 34598026 | 34601845 | chr19 | 42189678 | 42192265 | chr8 | 25591978 | 25592719 |
| chr21 | 10353698 | 10355340 | chr19 | 58349909 | 58350753 | chr12 | 34314640 | 34315312 |
| chr5 | 8840275 | 8840939 | chr20 | 37670306 | 37670743 | chr4 | 6127826 | 6128206 |
| chr6 | 163971805 | 163972191 | chr22 | 22198431 | 22200765 | chr1 | 7751905 | 7754331 |
| chr15 | 100131913 | 100132231 | chr12 | 127607292 | 127607859 | chr10 | 6470360 | 6472351 |
| chr16 | 1718907 | 1720773 | chr18 | 78670155 | 78671262 | chr10 | 131847065 | 131847517 |
| chr6 | 27217697 | 27218645 | chr1 | 38136198 | 38136520 | chr2 | 232480024 | 232480459 |
| chr2 | 192193295 | 192193621 | chr22 | 44134103 | 44135210 | chr22 | 47706271 | 47706688 |
| chr17 | 48148154 | 48149304 | chr4 | 132227437 | 132228059 | chr8 | 27957086 | 27957705 |
| chr11 | 71499098 | 71499302 | chr16 | 26050113 | 26051120 | chr11 | 133919408 | 133920169 |
| chr8 | 81599164 | 81601218 | chr8 | 4227192 | 4229172 | chr16 | 84838549 | 84839089 |
| chr9 | 133583563 | 133583868 | chr14 | 93777906 | 93778907 | chr8 | 2701838 | 2703847 |
| chr15 | 74955631 | 74955978 | chr5 | 43033866 | 43034259 | chr6 | 137001707 | 137002614 |
| chr16 | 86199520 | 86200960 | chr5 | 141360701 | 141361359 | chr16 | 51773278 | 51773682 |
| chr16 | 691747 | 692427 | chr14 | 100606233 | 100606692 | chr20 | 47497837 | 47499241 |
| chr6 | 142845029 | 142846142 | chr15 | 52858092 | 52858824 | chr16 | 34705620 | 34705988 |
| chr2 | 47714455 | 47716522 | chr3 | 134928152 | 134929757 | chr16 | 89342286 | 89342552 |
| chr5 | 1315700 | 1316316 | chr1 | 225910908 | 225911978 | chr19 | 37790579 | 37791102 |
| chr8 | 66056862 | 66057118 | chr4 | 32748206 | 32748725 | chr19 | 38302906 | 38304024 |
| chr8 | 140048895 | 140049422 | chr3 | 156252002 | 156254790 | chr20 | 21504460 | 21504930 |
| chr7 | 1210864 | 1211558 | chr12 | 119412932 | 119413751 | chr1 | 34171623 | 34172985 |
| chr7 | 2478619 | 2479381 | chr6 | 5828492 | 5829171 | chr17 | 46790780 | 46790970 |
| chr6 | 89887554 | 89888020 | chr4 | 3723229 | 3724485 | chr16 | 48722694 | 48725193 |
| chr19 | 974444 | 975102 | chr22 | 37421326 | 37421888 | chr6 | 6893863 | 6894260 |
| chr12 | 3274197 | 3275229 | chr9 | 126683686 | 126684556 | chr17 | 73976754 | 73977337 |
| chr18 | 61554279 | 61554752 | chr16 | 87703174 | 87703599 | chr11 | 1732153 | 1733186 |
| chr1 | 29542095 | 29542963 | chr1 | 181569689 | 181571275 | chr8 | 141523081 | 141523529 |
| chr13 | 41539746 | 41540598 | chr8 | 996374 | 996933 | chr6 | 73348926 | 73349595 |
| chr14 | 105860831 | 105861541 | chr12 | 107400533 | 107401071 | chr17 | 81406560 | 81407265 |
| chr1 | 62286520 | 62287322 | chr20 | 57754716 | 57755559 | chr8 | 3203189 | 3205278 |
| chr8 | 25935498 | 25936337 | chr9 | 126694910 | 126695499 | chr10 | 123517517 | 123518798 |
| chr11 | 129617780 | 129618557 | chr19 | 50997519 | 50999308 | chr19 | 1544104 | 1544694 |
| chr20 | 57518820 | 57519787 | chr9 | 97555306 | 97555553 | chr8 | 142412543 | 142412999 |
| chr2 | 132161364 | 132162731 | chr1 | 157861965 | 157863688 | chr4 | 1050841 | 1051085 |
| chr8 | 46254046 | 46254492 | chr12 | 130219107 | 130220067 | chr19 | 1728346 | 1728640 |
| chr13 | 25931466 | 25931996 | chr20 | 64166554 | 64167274 | chr2 | 240999587 | 241000159 |
| chr8 | 46116133 | 46116558 | chr8 | 43547551 | 43547911 | chr17 | 34132215 | 34133606 |
| chr22 | 46600113 | 46600686 | chr5 | 175873625 | 175875085 | chr19 | 2199641 | 2200177 |
| chr19 | 12160264 | 12160875 | chr13 | 110199479 | 110200129 | chr12 | 128160731 | 128161702 |
| chr12 | 66770891 | 66773604 | chr14 | 93748367 | 93748787 | chr12 | 111154704 | 111155736 |
| chr22 | 36708240 | 36709235 | chr10 | 101286441 | 101287276 | chr5 | 2338972 | 2339153 |
| chr7 | 6991100 | 6991748 | chr2 | 102186947 | 102187636 | chr11 | 31796121 | 31796447 |
| chr1 | 17686894 | 17687673 | chr10 | 100668808 | 100669729 | chr17 | 2894417 | 2894988 |
| chr17 | 30067967 | 30068271 | chr2 | 230898869 | 230899723 | chr6 | 27505490 | 27505910 |
| chr22 | 44905191 | 44905777 | chr14 | 33559699 | 33560898 | chr1 | 121006604 | 121007113 |
| chr6 | 28691268 | 28693014 | chr1 | 247538718 | 247540252 | chr12 | 34289082 | 34289936 |
| chr12 | 110887701 | 110888038 | chr17 | 73773295 | 73774042 | chr10 | 96384447 | 96384905 |
| chr4 | 154780756 | 154781686 | chr7 | 151007086 | 151007755 | chr11 | 46296497 | 46296751 |
| chr19 | 9208161 | 9209933 | chr16 | 78784877 | 78786064 | chr5 | 2097884 | 2099682 |
| chr1 | 204696798 | 204697693 | chr5 | 141441791 | 141442395 | chr1 | 22429104 | 22429255 |
| chr3 | 13684424 | 13685241 | chr12 | 98720 | 99278 | chr13 | 98621752 | 98623238 |
| chr3 | 21750211 | 21751473 | chr10 | 93310046 | 93310611 | chr22 | 45964520 | 45965347 |
| chr9 | 126515373 | 126516171 | chr20 | 59191733 | 59192316 | chr4 | 10120987 | 10121820 |
| chr7 | 2680692 | 2681417 | chr8 | 139889707 | 139890078 | chr12 | 111276792 | 111277011 |
| chr15 | 38554159 | 38554832 | chr9 | 128879258 | 128880591 | chr12 | 117050984 | 117051750 |
| chr1 | 38475034 | 38476064 | chr3 | 37694203 | 37694645 | chr19 | 5359747 | 5360629 |
| chr7 | 767662 | 768105 | chr5 | 1386843 | 1387647 | chr19 | 8336079 | 8336330 |
| chr18 | 33818379 | 33820034 | chr2 | 2291909 | 2292299 | chr21 | 36714095 | 36716161 |
| chr1 | 12177968 | 12178846 | chr2 | 130328343 | 130328910 | chr16 | 34897339 | 34898030 |
| chr9 | 90964340 | 90964932 | chr7 | 460639 | 461074 | chr8 | 123645896 | 123647090 |
| chr6 | 168196898 | 168197270 | chr9 | 121597916 | 121598222 | chr19 | 40594433 | 40595165 |
| chr10 | 3010251 | 3011178 | chr21 | 34807601 | 34808452 | chr19 | 56101314 | 56101906 |
| chr20 | 58130648 | 58131409 | chr6 | 32082494 | 32083272 | chr19 | 52658982 | 52659464 |
| chr10 | 1631179 | 1631682 | chr19 | 5746093 | 5746522 | chr3 | 146161840 | 146163600 |
| chr20 | 5469224 | 5470385 | chr1 | 22626610 | 22627334 | chr4 | 4754153 | 4754910 |
| chr17 | 49860098 | 49860556 | chr15 | 88574526 | 88575060 | chr16 | 48435328 | 48436091 |
| chr17 | 74361426 | 74362643 | chr18 | 51151929 | 51152358 | chr17 | 61593239 | 61595734 |
| chr21 | 32978841 | 32979376 | chr6 | 28597903 | 28598302 | chr8 | 141531345 | 141531981 |
| chr20 | 47805860 | 47806387 | chr12 | 57456754 | 57458577 | chr7 | 101362232 | 101362566 |
| chr22 | 50061297 | 50061728 | chr21 | 45021051 | 45021334 | chr2 | 65381678 | 65383592 |
| chr17 | 79252555 | 79253701 | chr2 | 9140354 | 9142570 | chr10 | 99042207 | 99043345 |
| chr10 | 74813380 | 74813973 | chr10 | 130473881 | 130474297 | chr2 | 94596110 | 94596951 |
| chr4 | 3675358 | 3676028 | chr1 | 3968639 | 3969741 | chr22 | 40728722 | 40729223 |
| chr10 | 91883703 | 91883962 | chr11 | 381779 | 382492 | chr15 | 33448875 | 33449374 |
| chr15 | 100667816 | 100668108 | chr16 | 7408478 | 7409444 | chr13 | 30108780 | 30109157 |
| chr12 | 5440809 | 5441843 | chr19 | 55489875 | 55490848 | chr20 | 57088305 | 57089552 |
| chr18 | 8468991 | 8469730 | chr13 | 111469912 | 111470182 | chr5 | 177582012 | 177582246 |
| chr12 | 48966357 | 48966474 | chr11 | 8232584 | 8233438 | chr2 | 238523300 | 238523949 |
| chr12 | 124517131 | 124518003 | chr22 | 44698080 | 44699016 | chr2 | 91685698 | 91686718 |
| chr8 | 141450867 | 141451394 | chr17 | 8740816 | 8741477 | chr19 | 5412521 | 5413181 |
| chr22 | 17799430 | 17801031 | chr19 | 50137116 | 50137318 | chr19 | 22574834 | 22576453 |
| chr2 | 131298987 | 131299945 | chr8 | 22286822 | 22287787 | chr21 | 9738392 | 9739205 |
| chr4 | 145637976 | 145638317 | chr2 | 113158247 | 113159947 | chr20 | 48734508 | 48735416 |
| chr12 | 117494953 | 117496953 | chr16 | 3030135 | 3030774 | chr5 | 3428419 | 3428743 |
| chr9 | 38016631 | 38017512 | chr16 | 54585119 | 54586223 | chr20 | 49557419 | 49559418 |
| chr7 | 101231786 | 101232318 | chr10 | 6171556 | 6172140 | chr6 | 127475301 | 127476012 |
| chr10 | 21499705 | 21500305 | chr1 | 22331654 | 22331891 | chr16 | 86281607 | 86282310 |
| chr11 | 122997660 | 122998738 | chr10 | 133030462 | 133031270 | chr17 | 58193794 | 58194167 |
| chr11 | 120556675 | 120557300 | chr15 | 98420225 | 98420999 | chr5 | 141400025 | 141401114 |
| chr16 | 15141048 | 15141445 | chr9 | 38478585 | 38478905 | chr2 | 216625835 | 216626485 |
| chr13 | 99575096 | 99575721 | chr7 | 1713145 | 1713734 | chr2 | 44829026 | 44829611 |
| chr17 | 79126124 | 79126742 | chr19 | 8977370 | 8978923 | chr17 | 79671090 | 79671870 |
| chr10 | 122123961 | 122125529 | chr11 | 132145988 | 132146427 | chr17 | 79993019 | 79993764 |
| chr22 | 49716160 | 49716830 | chr2 | 241519523 | 241520066 | chr6 | 9606918 | 9607514 |
| chr9 | 136189207 | 136189828 | chr1 | 3268207 | 3268508 | chr1 | 36939410 | 36941053 |
| chr2 | 130336749 | 130337637 | chr4 | 907735 | 908306 | chr1 | 46991380 | 46991979 |
| chr12 | 54080253 | 54080522 | chr18 | 47525976 | 47526586 | chr17 | 18568656 | 18569457 |
| chr12 | 34326898 | 34327629 | chr12 | 130339721 | 130340376 | chr7 | 5182866 | 5183468 |
| chr7 | 142786529 | 142787414 | chr11 | 300172 | 301462 | chr15 | 78926485 | 78927155 |
| chr2 | 31803755 | 31804118 | chr2 | 72137735 | 72138549 | chr1 | 47458834 | 47459478 |
| chr11 | 44308691 | 44309051 | chr16 | 49021132 | 49021875 | chr17 | 79098023 | 79098434 |
| chr8 | 46419883 | 46420903 | chr11 | 32430095 | 32430739 | chr7 | 1268996 | 1269664 |
| chr4 | 6116206 | 6117322 | chr5 | 5306873 | 5307558 | chr10 | 23157524 | 23159111 |
| chr11 | 116533362 | 116534574 | chr19 | 4397131 | 4398233 | chr20 | 63247915 | 63248402 |
| chr12 | 131459875 | 131461111 | chr22 | 26978992 | 26980845 | chr15 | 89486144 | 89487122 |
| chr1 | 148378964 | 148379525 | chr13 | 27808101 | 27809379 | chr17 | 45828276 | 45828999 |
| chr1 | 187767402 | 187768782 | chr11 | 57499120 | 57500207 | chr22 | 49705138 | 49706963 |
| chr6 | 30443820 | 30444646 | chr2 | 88003101 | 88003639 | chr1 | 4463740 | 4464314 |
| chr6 | 167145891 | 167147110 | chr1 | 46545636 | 46546886 | chr3 | 127157125 | 127157900 |
| chr2 | 86808906 | 86809548 | chr11 | 60331879 | 60335226 | chr17 | 13548987 | 13549549 |
| chr1 | 231495885 | 231496270 | chr14 | 104291493 | 104291923 | chr2 | 10880521 | 10881191 |
| chr3 | 11196172 | 11198239 | chr2 | 218893780 | 218894905 | chr22 | 27673876 | 27674418 |
| chr1 | 4637614 | 4638286 | chr9 | 137233886 | 137234860 | chr14 | 96728051 | 96729376 |
| chr12 | 15349973 | 15352575 | chr20 | 63534746 | 63535472 | chr18 | 78769003 | 78769651 |
| chr16 | 73796551 | 73797023 | chr20 | 30816241 | 30816648 | chr17 | 20229108 | 20229543 |
| chr10 | 72274810 | 72276692 | chr8 | 69581638 | 69583631 | chr16 | 49237048 | 49240175 |
| chr7 | 155408869 | 155409807 | chr12 | 14582401 | 14582539 | chr19 | 1395423 | 1396159 |
| chr10 | 132162261 | 132163661 | chr21 | 36658070 | 36659774 | chr17 | 28467659 | 28468737 |
| chr6 | 27685004 | 27685689 | chr15 | 79316952 | 79318552 | chr13 | 106472146 | 106472455 |
| chr2 | 240291404 | 240292182 | chr14 | 100817134 | 100817747 | chr10 | 102409472 | 102409977 |
| chr6 | 27230117 | 27230663 | chr11 | 2825487 | 2825814 | chr7 | 99094848 | 99096164 |
| chr1 | 22285742 | 22286267 | chr14 | 77206999 | 77208713 | chr17 | 50076491 | 50076997 |
| chr19 | 13055925 | 13056620 | chr12 | 130550129 | 130552949 | chr6 | 168648808 | 168649593 |
| chr17 | 74175423 | 74176696 | chr9 | 129752242 | 129752632 | chr19 | 5263703 | 5263946 |
| chr17 | 42990877 | 42991523 | chr1 | 181726876 | 181727704 | chr5 | 42923863 | 42924555 |
| chr2 | 236374508 | 236376989 | chr20 | 36411139 | 36412361 | chr9 | 86772377 | 86774256 |
| chr10 | 11849721 | 11850896 | chr19 | 9715596 | 9717458 | chr14 | 71982061 | 71983280 |
| chr10 | 1463141 | 1463536 | chr12 | 109822598 | 109823143 | chr11 | 125371110 | 125372110 |
| chr7 | 61020954 | 61022779 | chr17 | 78485002 | 78485417 | chr1 | 22339263 | 22339784 |
| chr12 | 196011 | 196766 | chr12 | 94732022 | 94732835 | chr7 | 40567 | 41223 |
| chr4 | 784091 | 785334 | chr1 | 11566607 | 11567003 | chr21 | 44302054 | 44302889 |
| chr16 | 4685216 | 4685924 | chr19 | 10293554 | 10294929 | chr19 | 16771021 | 16771541 |
| chr1 | 22739766 | 22740233 | chr1 | 1117425 | 1117690 | chr4 | 24795297 | 24795585 |
| chr19 | 1472869 | 1473411 | chr18 | 11823020 | 11825529 | chr3 | 122974722 | 122976026 |
| chr14 | 83091944 | 83092813 | chr15 | 57562020 | 57563555 | chr17 | 39740065 | 39740539 |
| chr7 | 127512763 | 127513843 | chr6 | 27302042 | 27302520 | chr15 | 97652827 | 97653103 |
| chr10 | 101142755 | 101145322 | chr1 | 16152821 | 16153498 | chr16 | 34133815 | 34134254 |
| chr20 | 2805414 | 2805900 | chr11 | 1555926 | 1556592 | chr22 | 33756722 | 33757436 |
| chr19 | 17393696 | 17394700 | chr17 | 74194490 | 74195058 | chr12 | 111216545 | 111217401 |
| chr3 | 8741497 | 8742137 | chr6 | 40195593 | 40195949 | chr1 | 2025634 | 2026181 |
| chr9 | 23682448 | 23684309 | chr6 | 28826849 | 28829732 | chr1 | 54990566 | 54991150 |
| chr7 | 35967521 | 35967933 | chr10 | 133238155 | 133238738 | chr17 | 73365193 | 73366178 |
| chr8 | 142951816 | 142952734 | chr1 | 41484301 | 41485515 | chr8 | 141764771 | 141765358 |
| chr1 | 180252995 | 180253819 | chr4 | 97403631 | 97404008 | chr1 | 5728039 | 5728782 |
| chr20 | 62486652 | 62487513 | chr3 | 149474349 | 149477100 | chr12 | 128142671 | 128144998 |
| chr9 | 136717488 | 136717908 | chr18 | 78813458 | 78814679 | chr15 | 40102846 | 40103866 |
| chr10 | 35574748 | 35575926 | chr22 | 41048811 | 41049279 | chr22 | 23245278 | 23245769 |
| chr1 | 2969658 | 2970089 | chr11 | 120147519 | 120148493 | chr2 | 219269334 | 219270810 |
| chr9 | 79439098 | 79439537 | chr20 | 51013909 | 51014734 | chr7 | 128270808 | 128271638 |
| chr8 | 11613516 | 11613847 | chr1 | 26155653 | 26156424 | chr16 | 89394488 | 89395382 |
| chr10 | 118473739 | 118474022 | chr1 | 26945415 | 26946148 | chr8 | 6607790 | 6608331 |
| chr3 | 184559103 | 184559704 | chr7 | 138380106 | 138382642 | chr2 | 20524984 | 20525548 |
| chr11 | 64438853 | 64439359 | chr1 | 112710820 | 112711458 | chr8 | 141541848 | 141542919 |
| chr20 | 63735547 | 63737112 | chr17 | 79919561 | 79920234 | chr2 | 10913944 | 10914539 |
| chr22 | 49355383 | 49355747 | chr12 | 34349505 | 34349939 | chr11 | 62687948 | 62688473 |
| chr9 | 133274266 | 133274795 | chr19 | 9061041 | 9061406 | chr9 | 135700241 | 135700864 |
| chr3 | 42265701 | 42266203 | chr10 | 2167364 | 2168659 | chr20 | 51122247 | 51123530 |
| chr5 | 16666174 | 16667087 | chr1 | 228098670 | 228098887 | chr1 | 11429389 | 11430607 |
| chr2 | 197164418 | 197165230 | chr10 | 1670541 | 1670927 | chr11 | 68750023 | 68750581 |
| chr10 | 114075607 | 114076308 | chr22 | 19162332 | 19163360 | chr17 | 22435567 | 22435839 |
| chr9 | 134176083 | 134176633 | chr3 | 128523733 | 128524252 | chr11 | 68992643 | 68993088 |
| chr12 | 1934799 | 1935516 | chr1 | 201374365 | 201374920 | chr5 | 402709 | 403462 |
| chr3 | 126288601 | 126289661 | chr11 | 125157989 | 125160215 | chr2 | 233754916 | 233755205 |
| chr14 | 100896477 | 100896889 | chr9 | 114156701 | 114157261 | chr19 | 1284072 | 1284666 |
| chr8 | 57259789 | 57260265 | chr5 | 156004950 | 156005890 | chr1 | 2466813 | 2467613 |
| chr12 | 125412169 | 125412633 | chr7 | 50782026 | 50783197 | chr11 | 45900869 | 45901255 |
| chr22 | 32416738 | 32418598 | chr4 | 153205131 | 153205610 | chr12 | 130203130 | 130203533 |
| chr2 | 91589408 | 91590250 | chr21 | 10732916 | 10733367 | chr5 | 176697932 | 176698546 |
| chr6 | 793655 | 795166 | chr15 | 53810167 | 53812028 | chr1 | 167564265 | 167566895 |
| chr2 | 126885128 | 126885977 | chr11 | 61265497 | 61266024 | chr7 | 55156946 | 55157452 |
| chr20 | 31744324 | 31745454 | chr1 | 14868924 | 14869591 | chr11 | 3619309 | 3620371 |
| chr9 | 133153027 | 133153692 | chr11 | 133541260 | 133542730 | chr5 | 2685437 | 2686603 |
| chr9 | 126206004 | 126206415 | chr2 | 118807279 | 118808252 | chr15 | 63018465 | 63020102 |
| chr6 | 27236231 | 27237206 | chr5 | 172637217 | 172637926 | chr10 | 117156912 | 117159028 |
| chr7 | 131909214 | 131909772 | chr15 | 83861559 | 83863286 | chr6 | 163944232 | 163945287 |
| chr11 | 70665849 | 70666736 | chr16 | 1305851 | 1306699 | chr17 | 5499652 | 5500367 |
| chr7 | 150969168 | 150970431 | chr17 | 49262137 | 49262549 | chr1 | 34697138 | 34698262 |
| chr8 | 123204879 | 123205754 | chr16 | 88445375 | 88445781 | chr6 | 33770087 | 33770740 |
| chr1 | 58624538 | 58625923 | chr5 | 163613599 | 163615857 | chr16 | 86074479 | 86075022 |
| chr11 | 49048470 | 49049151 | chr5 | 969358 | 969906 | chr10 | 127738259 | 127738956 |
| chr16 | 36175459 | 36177286 | chr7 | 90153505 | 90153957 | chr6 | 147452957 | 147453290 |
| chr3 | 186824500 | 186825117 | chr8 | 46918252 | 46918685 | chr22 | 48485559 | 48486402 |
| chr17 | 73341837 | 73342528 | chr1 | 155071568 | 155072296 | chr20 | 19532161 | 19532725 |
| chr16 | 1194758 | 1195268 | chr1 | 8898735 | 8899042 | chr21 | 31938264 | 31938945 |
| chr19 | 34480400 | 34480998 | chr12 | 119804098 | 119804591 | chr1 | 228635541 | 228635642 |
| chr11 | 69771777 | 69772919 | chr21 | 45378329 | 45378913 | chr19 | 401590 | 402441 |
| chr14 | 102059972 | 102060726 | chr1 | 22929288 | 22929477 | chr16 | 75833429 | 75836538 |
| chr13 | 30333985 | 30335957 | chr11 | 126452321 | 126452852 | chr15 | 68700654 | 68701351 |
| chr1 | 167127962 | 167128307 | chr3 | 197036379 | 197036687 | chr2 | 235236091 | 235237694 |
| chr2 | 76697552 | 76697777 | chr20 | 62356259 | 62356984 | chr19 | 2360755 | 2361921 |
| chr17 | 8315453 | 8315995 | chr6 | 40477750 | 40478119 | chr11 | 78902923 | 78903413 |
| chr9 | 130209156 | 130209674 | chr19 | 4552433 | 4553058 | chr8 | 4805360 | 4806115 |
| chr10 | 31585878 | 31586459 | chr5 | 177955263 | 177955834 | chr11 | 119863921 | 119864530 |
| chr21 | 10690297 | 10690918 | chr12 | 132178906 | 132179382 | chr9 | 113091192 | 113096729 |
| chr12 | 130496055 | 130496773 | chr16 | 29285765 | 29286603 | chr2 | 85811511 | 85811728 |
| chr12 | 131331599 | 131333282 | chr2 | 241866569 | 241867337 | chr2 | 71085358 | 71086947 |
| chr18 | 8990391 | 8991587 | chr1 | 161368627 | 161370261 | chr6 | 27249822 | 27250721 |
| chr17 | 22409093 | 22409324 | chr17 | 41487169 | 41487889 | chr15 | 31954563 | 31957493 |
| chr22 | 19622098 | 19622791 | chr5 | 124198873 | 124199189 | chr4 | 18418239 | 18418757 |
| chr1 | 54096672 | 54098018 | chr1 | 18809611 | 18811152 | chr6 | 3364633 | 3365057 |
| chr2 | 24974977 | 24975989 | chr15 | 57591146 | 57592821 | chr1 | 228522175 | 228526423 |
| chr5 | 140024315 | 140024758 | chr22 | 24542948 | 24544029 | chr8 | 46333452 | 46333975 |
| chr5 | 134414096 | 134414804 | chr7 | 44962699 | 44963438 | chr1 | 112143726 | 112145643 |
| chr1 | 58922215 | 58922634 | chr8 | 24943011 | 24943600 | chr4 | 5996051 | 5996588 |
| chr17 | 2927749 | 2928609 | chr14 | 50863674 | 50864095 | chr1 | 18945254 | 18945583 |
| chr21 | 36542028 | 36543218 | chr6 | 160371805 | 160373902 | chr12 | 48938308 | 48939541 |
| chr6 | 41491522 | 41492510 | chr17 | 49507562 | 49508280 | chr7 | 5143981 | 5144559 |
| chr2 | 127615464 | 127616516 | chr11 | 1927435 | 1928185 | chr19 | 20880732 | 20883370 |
| chr3 | 32821438 | 32821948 | chr6 | 28215282 | 28215874 | chr19 | 27679907 | 27682065 |
| chr6 | 149956309 | 149957367 | chr9 | 130668684 | 130669123 | chr19 | 17869338 | 17869801 |
| chr17 | 81991564 | 81992274 | chr6 | 27532035 | 27532344 | chr1 | 104407481 | 104407632 |
| chr9 | 28147639 | 28148140 | chr17 | 50781275 | 50781577 | chr8 | 20329375 | 20329676 |
| chr13 | 113939648 | 113940160 | chr6 | 15811474 | 15813239 | chr14 | 103727573 | 103727887 |
| chr11 | 63490507 | 63490790 | chr2 | 195797346 | 195799429 | chr17 | 81705555 | 81706120 |
| chr8 | 12994478 | 12994934 | chr1 | 10704403 | 10704970 | chr16 | 81180894 | 81181675 |
| chr15 | 26969459 | 26970333 | chr9 | 126489972 | 126490680 | chr19 | 41935534 | 41936082 |
| chr5 | 135477398 | 135477872 | chr12 | 71946735 | 71947961 | chr20 | 54619271 | 54620332 |
| chr8 | 1144545 | 1144967 | chr2 | 239661586 | 239662750 | chr21 | 43930149 | 43930563 |
| chr1 | 202073342 | 202073945 | chr1 | 13641748 | 13643468 | chr4 | 8747620 | 8748212 |
| chr7 | 157463179 | 157464575 | chr16 | 4906287 | 4906744 | chr17 | 79746833 | 79747266 |
| chr13 | 111388321 | 111389182 | chr2 | 78634169 | 78636761 | chr13 | 53200254 | 53200846 |
| chr20 | 61608159 | 61608983 | chr9 | 117032502 | 117037322 | chr3 | 39502053 | 39502546 |
| chr16 | 23713437 | 23713970 | chr1 | 53697902 | 53698652 | chr10 | 100809661 | 100810206 |
| chr6 | 27542056 | 27543898 | chr5 | 2170377 | 2171097 | chr1 | 5455963 | 5456555 |
| chr15 | 99029343 | 99029878 | chr8 | 946697 | 947159 | chr9 | 93061851 | 93062491 |
| chr1 | 207423617 | 207424218 | chr7 | 141916073 | 141918394 | chr2 | 70890079 | 70891565 |
| chr16 | 15143665 | 15144193 | chr11 | 120143778 | 120144437 | chr10 | 122352582 | 122353215 |
| chr11 | 20246246 | 20249619 | chr22 | 22195831 | 22196523 | chr10 | 132110759 | 132111087 |
| chr8 | 759450 | 760234 | chr4 | 2333483 | 2334257 | chr3 | 127337898 | 127338228 |
| chr6 | 39312524 | 39313041 | chr19 | 55438538 | 55439870 | chr15 | 26670300 | 26670928 |
| chr13 | 114229138 | 114230446 | chr9 | 92883820 | 92884259 | chr9 | 132202973 | 132203885 |
| chr4 | 185998989 | 186000211 | chr5 | 31732885 | 31733377 | chr1 | 42145869 | 42146753 |
| chr8 | 142507304 | 142508087 | chr16 | 15869435 | 15871119 | chr16 | 50906406 | 50906818 |
| chr12 | 54037466 | 54039473 | chr21 | 10716044 | 10717159 | chr2 | 172846346 | 172847538 |
| chr8 | 124232438 | 124233550 | chr1 | 243944286 | 243944892 | chr3 | 10366399 | 10367082 |
| chr10 | 78421168 | 78421973 | chr9 | 130925498 | 130926396 | chr7 | 41207121 | 41207347 |
| chr2 | 120331339 | 120332039 | chr16 | 36171086 | 36171617 | chr15 | 24675542 | 24675844 |
| chr7 | 155657154 | 155658824 | chr16 | 68643311 | 68643865 | chr17 | 77893401 | 77893835 |
| chr20 | 60393156 | 60393717 | chr21 | 9771002 | 9771807 | chr17 | 79127891 | 79128491 |
| chr2 | 121901865 | 121902545 | chr1 | 3708988 | 3710221 | chr10 | 28924647 | 28925095 |
| chr11 | 121372285 | 121373513 | chr1 | 6426558 | 6427167 | chr11 | 20164673 | 20166524 |
| chr13 | 100544202 | 100544892 | chr1 | 29960545 | 29961329 | chr20 | 62723292 | 62723528 |
| chr20 | 31484110 | 31484620 | chr16 | 86670160 | 86670558 | chr1 | 3172188 | 3172780 |
| chr1 | 3539929 | 3540491 | chr11 | 783125 | 784159 | chr19 | 13623275 | 13624333 |
| chr10 | 26946332 | 26946929 | chr10 | 101212060 | 101213300 | chr12 | 119004776 | 119006169 |
| chr2 | 28409248 | 28409556 | chr20 | 57022147 | 57023124 | chr10 | 30554004 | 30555110 |
| chr9 | 133656434 | 133657028 | chr2 | 236145574 | 236148063 | chr16 | 17562558 | 17563488 |
| chr21 | 10343764 | 10343953 | chr17 | 73903209 | 73903959 | chr1 | 119365656 | 119367882 |
| chr12 | 118126490 | 118127358 | chr1 | 211478935 | 211479323 | chr18 | 8703204 | 8703922 |
| chr11 | 113242792 | 113243412 | chr17 | 8226550 | 8227152 | chr20 | 63367261 | 63367810 |
| chr18 | 23664530 | 23665362 | chr20 | 29327668 | 29328807 | chr15 | 66073153 | 66073583 |
| chr12 | 34389943 | 34391895 | chr12 | 915840 | 916551 | chr12 | 47333680 | 47334486 |
| chr8 | 46230493 | 46231074 | chr1 | 5859889 | 5860173 | chr5 | 139754977 | 139755408 |
| chr16 | 68590959 | 68591311 | chr15 | 89380544 | 89382298 | chr17 | 830539 | 831107 |
| chr14 | 103274446 | 103274690 | chr3 | 46898086 | 46898663 | chr10 | 128618256 | 128619893 |
| chr20 | 51610687 | 51611355 | chr9 | 135192635 | 135193110 | chr5 | 1841377 | 1841667 |
| chr15 | 25062055 | 25062639 | chr16 | 73544628 | 73545151 | chr16 | 24450702 | 24451569 |
| chr1 | 227949103 | 227949790 | chr12 | 1644039 | 1644846 | chr3 | 58168373 | 58168678 |
| chr17 | 82375598 | 82376048 | chr18 | 75167726 | 75168879 | chr16 | 68364416 | 68364791 |
| chr5 | 12574063 | 12574789 | chr19 | 33733514 | 33734969 | chr1 | 50278652 | 50279476 |
| chr10 | 46911291 | 46911392 | chr3 | 138433838 | 138434235 | chr1 | 47711112 | 47711589 |
| chr2 | 232421427 | 232422030 | chr17 | 8310415 | 8310998 | chr18 | 80147169 | 80148338 |
| chr21 | 42780510 | 42781539 | chr10 | 103677866 | 103678478 | chr2 | 131574924 | 131575483 |
| chr20 | 62343059 | 62343546 | chr10 | 1212743 | 1213725 | chr19 | 13302333 | 13303058 |
| chr19 | 2228961 | 2229363 | chr12 | 124487488 | 124488238 | chr3 | 72470922 | 72473165 |
| chr4 | 4640769 | 4641404 | chr6 | 39061656 | 39062749 | chr2 | 2528651 | 2529228 |
| chr2 | 112562851 | 112563669 | chr19 | 18762315 | 18762686 | chr11 | 1119217 | 1119680 |
| chr4 | 16007314 | 16008782 | chr9 | 35036129 | 35036795 | chr5 | 42894119 | 42894498 |
| chr3 | 10944757 | 10946210 | chr22 | 19151114 | 19151476 | chr17 | 73610492 | 73611387 |
| chr7 | 25999747 | 25999968 | chr5 | 175770154 | 175770849 | chr17 | 76528436 | 76529518 |
| chr22 | 45877557 | 45878237 | chr19 | 674857 | 676061 | chr5 | 3103196 | 3103728 |
| chr11 | 1176310 | 1176830 | chr20 | 63129672 | 63131174 | chr10 | 70176060 | 70178128 |
| chr20 | 34310354 | 34311069 | chr10 | 69436007 | 69436367 | chr1 | 145460504 | 145460943 |
| chr7 | 135643942 | 135648492 | chr4 | 813802 | 814369 | chr7 | 31095984 | 31097165 |
| chr8 | 46143500 | 46144221 | chr14 | 35369888 | 35370388 | chr3 | 13567421 | 13568183 |
| chr1 | 5160945 | 5162356 | chr10 | 119222226 | 119223126 | chr20 | 46171782 | 46172815 |
| chr12 | 12071749 | 12072553 | chr6 | 33880526 | 33881105 | chr16 | 55760145 | 55761242 |
| chr18 | 24297508 | 24298645 | chr13 | 27974557 | 27975044 | chr21 | 36664739 | 36665409 |
| chr19 | 39307549 | 39308028 | chr2 | 9473653 | 9473907 | chr19 | 42314243 | 42314750 |
| chr12 | 8635454 | 8636201 | chr1 | 3067023 | 3067433 | chr9 | 105919 | 106691 |
| chr8 | 22775650 | 22776144 | chr1 | 118252663 | 118256286 | chr12 | 63821799 | 63822210 |
| chr11 | 26994046 | 26994587 | chr1 | 18554527 | 18555441 | chr12 | 4293636 | 4294187 |
| chr7 | 73342850 | 73343241 | chr18 | 22254143 | 22254615 | chr21 | 43731992 | 43732340 |
| chr2 | 153508938 | 153509422 | chr2 | 100843112 | 100844565 | chr1 | 39728240 | 39729371 |
| chr12 | 130494272 | 130495348 | chr3 | 126538434 | 126539010 | chr12 | 34296880 | 34298001 |
| chr20 | 36395559 | 36397399 | chr17 | 35699970 | 35703129 | chr20 | 25165771 | 25168934 |
| chr11 | 123260043 | 123262204 | chr16 | 30009181 | 30009697 | chr20 | 32228018 | 32228439 |
| chr3 | 308871 | 309594 | chr16 | 88265534 | 88265993 | chr8 | 12697297 | 12698090 |
| chr5 | 77181623 | 77181986 | chr16 | 34905701 | 34906045 | chr17 | 73308037 | 73308368 |
| chr17 | 81341865 | 81342048 | chr7 | 25568652 | 25569180 | chr10 | 10709702 | 10711837 |
| chr13 | 27481061 | 27482113 | chr16 | 5607137 | 5607975 | chr22 | 41912322 | 41913333 |
| chr14 | 54831769 | 54832168 | chr17 | 8392305 | 8392654 | chr19 | 39914944 | 39915489 |
| chr12 | 130776129 | 130776498 | chr7 | 25069699 | 25070729 | chr19 | 6982022 | 6984580 |
| chr6 | 30892200 | 30892753 | chr20 | 22662737 | 22663528 | chr9 | 41236922 | 41237248 |
| chr20 | 57877237 | 57877760 | chr17 | 45136509 | 45137581 | chr2 | 172107398 | 172107853 |
| chr21 | 10587579 | 10588010 | chr16 | 46901655 | 46902212 | chr17 | 77789942 | 77790664 |
| chr5 | 175728369 | 175728967 | chr20 | 63173764 | 63174483 | chr22 | 22163804 | 22165322 |
| chr1 | 8347699 | 8348041 | chr1 | 46479830 | 46480044 | chr17 | 6242567 | 6243184 |
| chr19 | 55377749 | 55378855 | chr16 | 69932189 | 69934691 | chr9 | 137025388 | 137025908 |
| chr16 | 82891304 | 82893902 | chr3 | 122661768 | 122662436 | chr16 | 10829597 | 10831189 |
| chr2 | 73260341 | 73260881 | chr9 | 137174816 | 137175536 | chr3 | 125148647 | 125149255 |
| chr16 | 86953831 | 86954600 | chr10 | 7056315 | 7057144 | chr1 | 19112662 | 19113157 |
| chr10 | 98326574 | 98327092 | chr16 | 77435642 | 77437266 | chr12 | 9739655 | 9740380 |
| chr10 | 2497277 | 2498156 | chr11 | 74696617 | 74697228 | chr7 | 28227132 | 28228440 |
| chr3 | 67154134 | 67154913 | chr1 | 156455534 | 156456125 | chr18 | 75638282 | 75638869 |
| chr1 | 109526362 | 109528178 | chr18 | 12247043 | 12247664 | chr22 | 26117077 | 26119141 |
| chr11 | 127103949 | 127106159 | chr1 | 226884735 | 226885669 | chr10 | 130254350 | 130254838 |
| chr12 | 111025026 | 111025942 | chr3 | 169813364 | 169814262 | chr10 | 71035843 | 71038590 |
| chr9 | 126298111 | 126298681 | chr19 | 14550189 | 14550502 | chr16 | 71440370 | 71441024 |
| chr12 | 132243558 | 132244107 | chr15 | 74256117 | 74256701 | chr7 | 74454746 | 74455252 |
| chr17 | 77696489 | 77697591 | chr4 | 147688814 | 147689600 | chr10 | 15460134 | 15462083 |
| chr17 | 76833877 | 76834857 | chr6 | 34115731 | 34116155 | chr9 | 127526776 | 127527337 |
| chr16 | 15053652 | 15054390 | chr22 | 48901248 | 48901689 | chr2 | 136025423 | 136025882 |
| chr1 | 30247386 | 30247936 | chr5 | 134262717 | 134264295 | chr17 | 78040594 | 78041201 |
| chr16 | 49464979 | 49465803 | chr10 | 23338214 | 23338780 | chr17 | 74109736 | 74110173 |
| chr19 | 3739001 | 3739447 | chr2 | 240704664 | 240704857 | chr22 | 26975670 | 26977453 |
| chr4 | 3747935 | 3748594 | chr3 | 10467544 | 10468938 | chr10 | 13773265 | 13774006 |
| chr22 | 20149718 | 20150173 | chr6 | 37061121 | 37061820 | chr1 | 17347751 | 17348804 |
| chr19 | 4276684 | 4277337 | chr3 | 184491308 | 184491634 | chr4 | 3874020 | 3874339 |
| chr10 | 126613779 | 126615255 | chr8 | 22548834 | 22549613 | chr15 | 98524559 | 98525849 |
| chr15 | 67477176 | 67477390 | chr22 | 31127100 | 31127966 | chr10 | 5646416 | 5646795 |
| chr2 | 108944861 | 108945211 | chr3 | 126259416 | 126259999 | chr11 | 126112882 | 126114084 |
| chr14 | 106324106 | 106324505 | chr19 | 2341782 | 2342422 | chr16 | 1067899 | 1068352 |
| chr6 | 27682340 | 27683815 | chr4 | 1116094 | 1120214 | chr2 | 220748413 | 220749632 |
| chr16 | 49266514 | 49269844 | chr19 | 50775037 | 50776258 | chr5 | 141494693 | 141495292 |
| chr5 | 45311823 | 45315060 | chr9 | 126421836 | 126422741 | chr20 | 46282293 | 46283205 |
| chr17 | 77801825 | 77802971 | chr6 | 164967137 | 164967395 | chr6 | 27275648 | 27276733 |
| chr16 | 79856609 | 79856829 | chr11 | 72242956 | 72243464 | chr11 | 1747859 | 1748233 |
| chr7 | 1123762 | 1124081 | chr7 | 155113562 | 155114735 | chr22 | 44782251 | 44782910 |
| chr4 | 54539450 | 54540290 | chr12 | 132608150 | 132609030 | chr5 | 135508430 | 135508703 |
| chr2 | 23526450 | 23527103 | chr2 | 205763631 | 205764050 | chr14 | 97651706 | 97653214 |
| chr18 | 74368890 | 74369361 | chr13 | 109733976 | 109734375 | chr19 | 35830859 | 35831160 |
| chr8 | 109641896 | 109642996 | chr22 | 32042961 | 32043390 | chr10 | 38512990 | 38515360 |
| chr2 | 74888854 | 74889415 | chr9 | 135609267 | 135609840 | chr5 | 3152998 | 3155119 |
| chr4 | 4863041 | 4864098 | chr4 | 7312708 | 7313700 | chr13 | 27787266 | 27789239 |
| chr8 | 6880787 | 6881631 | chr22 | 26952583 | 26953085 | chr6 | 11609647 | 11610476 |
| chr11 | 68845898 | 68846597 | chr15 | 42156011 | 42156441 | chr11 | 126996903 | 126997680 |
| chr17 | 42653522 | 42654118 | chr19 | 48860355 | 48860677 | chr17 | 68474381 | 68474692 |
| chr2 | 89789298 | 89789722 | chr6 | 90177182 | 90178994 | chr12 | 118798140 | 118799622 |
| chr14 | 93952930 | 93953748 | chr10 | 42835947 | 42836317 | chr17 | 34538997 | 34540324 |
| chr19 | 19514085 | 19514783 | chr4 | 57194175 | 57195046 | chr21 | 10666417 | 10667314 |
| chr3 | 179942415 | 179942714 | chr12 | 49338634 | 49341100 | chr5 | 1650762 | 1651321 |
| chr16 | 2511172 | 2511656 | chr2 | 72955050 | 72955472 | chr11 | 70443695 | 70444022 |
| chr16 | 89048122 | 89048667 | chr20 | 62992512 | 62992911 | chr2 | 239495886 | 239496639 |
| chr2 | 240984165 | 240984455 | chr17 | 77266986 | 77267475 | chr14 | 77000994 | 77001480 |
| chr19 | 50637944 | 50638614 | chr12 | 34353588 | 34354516 | chr22 | 23536883 | 23537513 |
| chr8 | 30912929 | 30913248 | chr2 | 98783243 | 98785894 | chr5 | 141874215 | 141875301 |
| chr5 | 37372 | 39553 | chr17 | 31851646 | 31852208 | chr10 | 18400338 | 18401318 |
| chr17 | 29609028 | 29609394 | chr1 | 11388651 | 11389232 | chr11 | 1426528 | 1427078 |
| chr5 | 3787137 | 3789288 | chr3 | 107697569 | 107698638 | chr4 | 14862936 | 14863264 |
| chr16 | 77338380 | 77340084 | chr20 | 63411912 | 63412544 | chr1 | 163480090 | 163481070 |
| chr3 | 12941855 | 12942378 | chr16 | 85168129 | 85168722 | chr15 | 88708546 | 88709293 |
| chr5 | 55169835 | 55171692 | chr3 | 177270748 | 177271073 | chr14 | 91364983 | 91365860 |
| chr17 | 34992316 | 34993043 | chr1 | 4881611 | 4882455 | chr1 | 213905357 | 213905599 |
| chr10 | 22477462 | 22477933 | chr14 | 96698734 | 96699218 | chr11 | 131542854 | 131543972 |
| chr10 | 23185679 | 23187337 | chr14 | 100827640 | 100828029 | chr9 | 109559720 | 109560886 |
| chr1 | 159793662 | 159795636 | chr10 | 100753335 | 100755360 | chr2 | 94266807 | 94267529 |
| chr11 | 44337391 | 44338451 | chr14 | 106656992 | 106658261 | chr11 | 50260884 | 50261325 |
| chr2 | 235605485 | 235606222 | chr17 | 11637530 | 11638546 | chr8 | 141978993 | 141979780 |
| chr1 | 24970017 | 24970559 | chr11 | 57234235 | 57235256 | chr18 | 78292250 | 78293624 |
| chr12 | 132491556 | 132494129 | chr5 | 81833844 | 81834553 | chr20 | 61965097 | 61965691 |
| chr22 | 37660947 | 37662537 | chr22 | 44543131 | 44543828 | chr11 | 131760510 | 131760886 |
| chr8 | 30731149 | 30731748 | chr12 | 117214185 | 117215288 | chr22 | 25395561 | 25396037 |
| chr6 | 28740418 | 28741345 | chr16 | 86739880 | 86740673 | chr9 | 124125916 | 124126685 |
| chr1 | 150283433 | 150283869 | chr1 | 247347822 | 247348348 | chr13 | 23847980 | 23849861 |
| chr19 | 4207556 | 4208364 | chr18 | 36440431 | 36440926 | chr18 | 8526006 | 8526805 |
| chr16 | 25915041 | 25916449 | chr2 | 163348112 | 163349807 | chr10 | 25931384 | 25933326 |
| chr20 | 35174354 | 35175160 | chr1 | 121048408 | 121051402 | chr13 | 106316788 | 106317680 |
| chr10 | 122054873 | 122055390 | chr10 | 1267441 | 1268033 | chr2 | 239440099 | 239440986 |
| chr9 | 134181576 | 134182070 | chr12 | 33055856 | 33056388 | chr10 | 127905827 | 127906357 |
| chr1 | 54856320 | 54857436 | chr19 | 38428407 | 38429383 | chr15 | 66078741 | 66079436 |
| chr19 | 1699419 | 1700740 | chr2 | 10406584 | 10408024 | chr19 | 41813124 | 41814141 |
| chr16 | 26057480 | 26058878 | chr14 | 85442886 | 85444818 | chr17 | 79482732 | 79483885 |
| chr5 | 139480848 | 139481673 | chr7 | 27145093 | 27145573 | chr12 | 116443794 | 116444571 |
| chr17 | 29069663 | 29070243 | chr12 | 34322512 | 34323434 | chr4 | 1225079 | 1225444 |
| chr22 | 29703884 | 29704936 | chr3 | 139043975 | 139044633 | chr17 | 79654125 | 79654937 |
| chr5 | 97527699 | 97528021 | chr19 | 29839245 | 29839775 | chr11 | 126828621 | 126831288 |
| chr6 | 12549322 | 12549959 | chr11 | 133895135 | 133896074 | chr5 | 140860761 | 140861009 |
| chr10 | 133465316 | 133465813 | chr9 | 130303686 | 130304029 | chr6 | 30462622 | 30463424 |
| chr6 | 34056031 | 34056702 | chr12 | 113765989 | 113767141 | chr21 | 44391291 | 44391528 |
| chr6 | 13014284 | 13014602 | chr17 | 79382079 | 79382661 | chr14 | 93899129 | 93899764 |
| chr20 | 43215855 | 43217019 | chr8 | 69711112 | 69712315 | chr3 | 194984939 | 194985553 |
| chr14 | 101068562 | 101069139 | chr18 | 79847217 | 79847566 | chr4 | 13482919 | 13483402 |
| chr10 | 2649072 | 2649720 | chr8 | 135458498 | 135458910 | chr11 | 1082685 | 1083291 |
| chr20 | 53412965 | 53413446 | chr16 | 86580126 | 86581275 | chr17 | 47778349 | 47779055 |
| chr17 | 44337127 | 44337334 | chr19 | 15180716 | 15181590 | chr20 | 17643027 | 17643440 |
| chr20 | 61687022 | 61687678 | chr7 | 112622065 | 112622728 | chr14 | 20659719 | 20660370 |
| chr6 | 73299064 | 73299906 | chr14 | 94689981 | 94690655 | chr7 | 158328299 | 158329051 |
| chr7 | 149188966 | 149189542 | chr7 | 52753872 | 52754900 | chr1 | 3716665 | 3717522 |
| chr3 | 13658404 | 13659428 | chr16 | 35500972 | 35501505 | chr12 | 3315056 | 3315775 |
| chr12 | 130657428 | 130658104 | chr10 | 132518492 | 132519100 | chr18 | 10439567 | 10440143 |
| chr21 | 41678677 | 41679548 | chr6 | 112325612 | 112326806 | chr13 | 111852555 | 111853343 |
| chr18 | 79877271 | 79877881 | chr19 | 51293066 | 51293642 | chr12 | 128146589 | 128147258 |
| chr15 | 66922196 | 66924302 | chr2 | 1708825 | 1709108 | chr17 | 80070966 | 80071286 |
| chr3 | 186595945 | 186596785 | chr18 | 59195531 | 59196954 | chr7 | 55212668 | 55213325 |
| chr16 | 88559913 | 88560474 | chr10 | 28502019 | 28503371 | chr14 | 72715066 | 72716195 |
| chr1 | 157278818 | 157279545 | chr2 | 239026894 | 239027346 | chr2 | 20662996 | 20664618 |
| chr17 | 79400078 | 79400514 | chr19 | 21202024 | 21203673 | chr5 | 37913364 | 37915118 |
| chr7 | 128146885 | 128149817 | chr16 | 49801049 | 49801442 | chr7 | 55448686 | 55449078 |
| chr1 | 167511731 | 167512414 | chr17 | 1188675 | 1189169 | chr7 | 4871950 | 4872806 |
| chr3 | 157614223 | 157617289 | chr18 | 75130828 | 75131715 | chr7 | 139682690 | 139684488 |
| chr11 | 119791767 | 119794363 | chr15 | 71129763 | 71130407 | chr1 | 18729547 | 18730758 |
| chr1 | 1462333 | 1462883 | chr12 | 106588972 | 106591518 | chr21 | 31870619 | 31871589 |
| chr16 | 1365933 | 1366195 | chr7 | 159141953 | 159143580 | chr7 | 36028486 | 36029360 |
| chr5 | 174708568 | 174708973 | chr22 | 25798083 | 25798965 | chr2 | 131005218 | 131005754 |
| chr12 | 67067778 | 67068070 | chr9 | 38311358 | 38312020 | chr1 | 18681957 | 18682814 |
| chr15 | 82699634 | 82699991 | chr2 | 89811257 | 89811799 | chr3 | 16773548 | 16774076 |
| chr15 | 99106194 | 99106799 | chr2 | 235190661 | 235191745 | chr17 | 80850386 | 80851166 |
| chr13 | 113732699 | 113733001 | chr16 | 11678246 | 11678689 | chr9 | 70625085 | 70625714 |
| chr1 | 53685381 | 53685971 | chr16 | 70804292 | 70805467 | chr19 | 46259344 | 46260235 |
| chr17 | 6638094 | 6638406 | chr17 | 10980478 | 10982610 | chr10 | 30811336 | 30811873 |
| chr18 | 63940468 | 63941139 | chr8 | 94242179 | 94243748 | chr12 | 12787547 | 12788319 |
| chr14 | 70555552 | 70556420 | chr15 | 78339195 | 78339880 | chr16 | 20301845 | 20303505 |
| chr4 | 6561905 | 6562770 | chr5 | 1907932 | 1909309 | chr16 | 13431783 | 13434156 |
| chr9 | 134783869 | 134784753 | chr22 | 42314018 | 42314630 | chr5 | 1725807 | 1726057 |
| chr17 | 79114221 | 79115106 | chr6 | 28474717 | 28475390 | chr1 | 5337727 | 5339432 |
| chr9 | 91900195 | 91900487 | chr18 | 77790035 | 77792378 | chr8 | 46222701 | 46223725 |
| chr3 | 75655393 | 75656248 | chr19 | 1313484 | 1314109 | chr15 | 19969203 | 19971617 |
| chr11 | 32431643 | 32432277 | chr16 | 20072137 | 20073100 | chr7 | 101744065 | 101744376 |
| chr3 | 73101887 | 73103560 | chr22 | 41557304 | 41557824 | chr20 | 56830911 | 56832062 |
| chr12 | 54054978 | 54055992 | chr19 | 5189014 | 5189779 | chr22 | 27435756 | 27436043 |
| chr16 | 84506716 | 84507841 | chr4 | 7146214 | 7146899 | chr5 | 482208 | 482721 |
| chr4 | 180385718 | 180385941 | chr10 | 122165003 | 122165684 | chr11 | 69662470 | 69662888 |
| chr16 | 28020431 | 28022029 | chr7 | 92412385 | 92414635 | chr10 | 129394621 | 129394886 |
| chr2 | 60652642 | 60653207 | chr16 | 3413930 | 3414510 | chr15 | 92583434 | 92584204 |
| chr11 | 64164573 | 64165245 | chr20 | 22332688 | 22334088 | chr13 | 25095642 | 25096227 |
| chr17 | 72461306 | 72462917 | chr6 | 1473222 | 1475093 | chr1 | 245687876 | 245688555 |
| chr6 | 156396812 | 156397490 | chr5 | 4173110 | 4173907 | chr15 | 89443313 | 89444581 |
| chr12 | 127875868 | 127876104 | chr17 | 77931232 | 77931617 | chr12 | 124094474 | 124094978 |
| chr16 | 49968934 | 49969702 | chr5 | 175820486 | 175821249 | chr1 | 182683812 | 182684994 |
| chr1 | 33826121 | 33830288 | chr12 | 117512584 | 117514844 | chr8 | 6561917 | 6562774 |
| chr1 | 6961170 | 6961558 | chr7 | 142558257 | 142560219 | chr16 | 1919332 | 1919696 |
| chr16 | 66006201 | 66006930 | chr22 | 48255708 | 48256359 | chr20 | 62450242 | 62450750 |
| chr10 | 66926947 | 66927761 | chr7 | 132386062 | 132386671 | chr9 | 135346866 | 135347433 |
| chr10 | 72088948 | 72089410 | chr5 | 137497837 | 137498171 | chr7 | 50327974 | 50329891 |
| chr17 | 7715883 | 7716481 | chr1 | 116775027 | 116775610 | chr10 | 113557983 | 113558819 |
| chr20 | 24034206 | 24035063 | chr21 | 41115215 | 41117401 | chr1 | 156651429 | 156652127 |
| chr2 | 216670240 | 216670823 | chr9 | 36762113 | 36763587 | chr5 | 178498772 | 178501321 |
| chr14 | 104085861 | 104086591 | chr10 | 128480175 | 128481324 | chr10 | 16792422 | 16792959 |
| chr11 | 63962550 | 63963171 | chr17 | 27413793 | 27415880 | chr19 | 58054985 | 58055335 |
| chr8 | 79827098 | 79827784 | chr21 | 42376066 | 42376834 | chr17 | 80845226 | 80845792 |
| chr19 | 7551344 | 7551778 | chr3 | 11997226 | 11998554 | chr3 | 58660874 | 58661687 |
| chr16 | 15854561 | 15855200 | chr10 | 121292643 | 121293437 | chr19 | 1682814 | 1683839 |
| chr3 | 127404848 | 127405643 | chr7 | 64182252 | 64182712 | chr7 | 158031875 | 158032223 |
| chr13 | 113820790 | 113821279 | chr6 | 35410914 | 35411477 | chr16 | 23186181 | 23186685 |
| chr10 | 126332690 | 126332923 | chr17 | 1932120 | 1932785 | chr16 | 49336693 | 49337539 |
| chr2 | 27307860 | 27308623 | chr10 | 132146286 | 132146812 | chr21 | 44368959 | 44370425 |
| chr8 | 3057884 | 3060900 | chr15 | 28045442 | 28047616 | chr10 | 44293440 | 44293742 |
| chr1 | 18784007 | 18784734 | chr8 | 46954541 | 46954782 | chr9 | 6715320 | 6715858 |
| chr12 | 104655170 | 104655824 | chr15 | 28992336 | 28993217 | chr17 | 73867036 | 73867377 |
| chr19 | 5127688 | 5129136 | chr11 | 134715109 | 134716595 | chr10 | 122710157 | 122712704 |
| chr4 | 15881223 | 15882371 | chr5 | 34829345 | 34831095 | chr13 | 46385690 | 46386335 |
| chr22 | 11324671 | 11325531 | chr9 | 133563370 | 133563671 | chr19 | 5402187 | 5402913 |
| chr2 | 131646712 | 131647236 | chr2 | 110858289 | 110858728 | chr13 | 83584669 | 83585318 |
| chr12 | 119976444 | 119977569 | chr12 | 43735037 | 43735926 | chr4 | 6154856 | 6155343 |
| chr17 | 47872343 | 47873672 | chr11 | 21752685 | 21753178 | chr18 | 77366825 | 77367169 |
| chr10 | 116690840 | 116691937 | chr2 | 132347473 | 132347992 | chr4 | 8071897 | 8072106 |
| chr19 | 45216942 | 45217996 | chr14 | 99074761 | 99075213 | chr14 | 104474121 | 104474434 |
| chr1 | 6104982 | 6105536 | chr19 | 55006931 | 55009232 | chr13 | 112101532 | 112102262 |
| chr16 | 1208196 | 1208663 | chr14 | 105204202 | 105205134 | chr15 | 96420748 | 96421563 |
| chr2 | 1080523 | 1081527 | chr6 | 90611958 | 90612258 | chr3 | 39428687 | 39429406 |
| chr1 | 51845266 | 51845587 | chr20 | 61567189 | 61567811 | chr12 | 47566908 | 47569340 |
| chr19 | 50703428 | 50703798 | chr16 | 1883831 | 1884833 | chr9 | 88698983 | 88701000 |
| chr5 | 151238427 | 151240098 | chr20 | 64162395 | 64162819 | chr19 | 6677968 | 6678320 |
| chr5 | 180127068 | 180127548 | chr17 | 16666042 | 16666741 | chr9 | 135721167 | 135721710 |
| chr18 | 59208509 | 59211129 | chr11 | 6270619 | 6270992 | chr20 | 38336060 | 38336999 |
| chr20 | 50009833 | 50010134 | chr12 | 130460758 | 130461044 | chr10 | 133603912 | 133607060 |
| chr19 | 50464440 | 50464632 | chr19 | 50557184 | 50557916 | chr6 | 103332593 | 103333035 |
| chr8 | 140348815 | 140349864 | chr22 | 16717721 | 16718302 | chr13 | 27903082 | 27904797 |
| chr6 | 167517598 | 167518145 | chr1 | 245680023 | 245680835 | chr5 | 179265148 | 179265712 |
| chr16 | 1163452 | 1163695 | chr15 | 72795981 | 72796705 | chr10 | 2590712 | 2591815 |
| chr3 | 186662919 | 186665360 | chr19 | 45958610 | 45959349 | chr12 | 106739809 | 106741238 |
| chr20 | 25085664 | 25086601 | chr20 | 30281859 | 30283337 | chr9 | 98246259 | 98247466 |
| chr20 | 61733536 | 61734265 | chr11 | 133946530 | 133947071 | chr2 | 102620233 | 102620705 |
| chr6 | 26319495 | 26320121 | chr2 | 364229 | 365022 | chr14 | 103208197 | 103209288 |
| chr11 | 126081386 | 126082539 | chr16 | 73793385 | 73794567 | chr10 | 36817992 | 36818385 |
| chr5 | 113676816 | 113680193 | chr4 | 171814453 | 171814907 | chr2 | 98070198 | 98071196 |
| chr19 | 50234154 | 50234774 | chr19 | 610001 | 611514 | chr3 | 48588313 | 48588488 |
| chr17 | 49573867 | 49574372 | chr11 | 1130375 | 1131293 | chr22 | 48968414 | 48969012 |
| chr22 | 47623146 | 47623692 | chr20 | 51156079 | 51156853 | chr5 | 173364154 | 173367390 |
| chr17 | 67490586 | 67491773 | chr4 | 54624915 | 54626651 | chr7 | 52273796 | 52274978 |
| chr13 | 101218431 | 101222418 | chr2 | 227744244 | 227744722 | chr1 | 210294206 | 210295369 |
| chr11 | 114848116 | 114849347 | chr6 | 95551907 | 95552961 | chr3 | 65148366 | 65149737 |
| chr22 | 46465584 | 46466001 | chr8 | 33604566 | 33605163 | chr11 | 123188092 | 123188752 |
| chr2 | 105236743 | 105237595 | chr10 | 11012280 | 11013072 | chr19 | 45576645 | 45577990 |
| chr5 | 152106798 | 152110172 | chr1 | 4737880 | 4738962 | chr18 | 79599242 | 79599686 |
| chr2 | 131151714 | 131152745 | chr11 | 114092015 | 114092722 | chr11 | 28606961 | 28607868 |
| chr5 | 139475849 | 139476644 | chr15 | 89747253 | 89748174 | chr17 | 50154012 | 50154851 |
| chr16 | 28608863 | 28611069 | chr11 | 2443475 | 2443986 | chr11 | 45554553 | 45555457 |
| chr1 | 47712442 | 47713121 | chr5 | 36662654 | 36663795 | chr17 | 65620441 | 65621386 |
| chr12 | 132603747 | 132604854 | chr2 | 128901778 | 128902869 | chr8 | 27862494 | 27863707 |
| chr16 | 11400998 | 11401838 | chr6 | 51410301 | 51410781 | chr7 | 73769096 | 73769547 |
| chr19 | 55564348 | 55564562 | chr19 | 54344170 | 54346441 | chr3 | 32114218 | 32115057 |
| chr7 | 157581753 | 157582070 | chr5 | 172699554 | 172700938 | chr12 | 20093258 | 20093583 |
| chr20 | 61657524 | 61658202 | chr17 | 803212 | 804108 | chr20 | 46150837 | 46151558 |
| chr1 | 9250669 | 9251264 | chr13 | 107210460 | 107211353 | chr1 | 79006600 | 79007021 |
| chr15 | 60226872 | 60229106 | chr7 | 30913455 | 30913957 | chr1 | 3944692 | 3945196 |
| chr14 | 61111207 | 61111427 | chr11 | 399079 | 400433 | chr4 | 186733488 | 186734437 |
| chr1 | 30706684 | 30707581 | chr16 | 1430845 | 1431310 | chr11 | 77588679 | 77589141 |
| chr2 | 3885985 | 3886462 | chr20 | 62974245 | 62974760 | chr9 | 132488701 | 132489364 |
| chr2 | 74130019 | 74130793 | chr16 | 1979478 | 1979790 | chr9 | 134441009 | 134441533 |
| chr2 | 218877644 | 218879463 | chr16 | 85819959 | 85820931 | chr20 | 61435501 | 61436796 |
| chr11 | 2165413 | 2165930 | chr5 | 169302843 | 169303687 | chr1 | 75842656 | 75843397 |
| chr1 | 4083458 | 4084123 | chr21 | 44949648 | 44950683 | chr9 | 133614203 | 133615127 |
| chr22 | 10767229 | 10769113 | chr2 | 240370114 | 240370582 | chr12 | 108151125 | 108152449 |
| chr1 | 46533644 | 46534378 | chr20 | 29323829 | 29326555 | chr15 | 40770508 | 40771047 |
| chr10 | 21510048 | 21510984 | chr18 | 41763152 | 41764577 | chr4 | 6223619 | 6225475 |
| chr15 | 40863769 | 40864427 | chr20 | 56848003 | 56849658 | chr21 | 44982807 | 44983178 |
| chr3 | 187291291 | 187291788 | chr13 | 114166432 | 114166760 | chr19 | 4546863 | 4547571 |
| chr17 | 75025436 | 75026063 | chr12 | 34374842 | 34376276 | chr3 | 186258047 | 186259612 |
| chr9 | 16171757 | 16172337 | chr8 | 46144858 | 46145472 | chr8 | 6390382 | 6392448 |
| chr20 | 57835204 | 57835898 | chr1 | 7615682 | 7616566 | chr14 | 24801833 | 24802516 |
| chr22 | 32204626 | 32205853 | chr1 | 12078542 | 12079303 | chr20 | 44389226 | 44389953 |
| chr2 | 18584454 | 18585029 | chr14 | 103541825 | 103542886 | chr16 | 18009197 | 18011142 |
| chr7 | 152907891 | 152908149 | chr8 | 142448240 | 142448778 | chr18 | 75204562 | 75205910 |
| chr8 | 21767827 | 21768504 | chr11 | 71541271 | 71542112 | chr9 | 131619320 | 131620866 |
| chr9 | 97076251 | 97076954 | chr22 | 44795584 | 44796477 | chr2 | 3970815 | 3971215 |
| chr20 | 63473647 | 63473917 | chr20 | 24778645 | 24779066 | chr2 | 235323714 | 235325093 |
| chr17 | 36929103 | 36930085 | chr19 | 6261573 | 6262174 | chr4 | 7104061 | 7104817 |
| chr10 | 624762 | 625594 | chr12 | 114415741 | 114416357 | chr6 | 44456756 | 44457049 |
| chr1 | 20399224 | 20401086 | chr8 | 22626186 | 22627099 | chr13 | 112330946 | 112332050 |
| chr10 | 56350186 | 56351339 | chr5 | 181219420 | 181220045 | chr18 | 46433064 | 46433668 |
| chr19 | 55541312 | 55542148 | chr7 | 134831752 | 134832179 | chr5 | 128630826 | 128632747 |
| chr2 | 241560118 | 241560546 | chr4 | 76801956 | 76802513 | chr18 | 79526792 | 79527287 |
| chr1 | 41423046 | 41424328 | chr7 | 151254523 | 151255092 | chr17 | 58136368 | 58137972 |
| chr17 | 79103515 | 79104263 | chr17 | 9532210 | 9533489 | chr17 | 81094806 | 81095389 |
| chr6 | 27213189 | 27214331 | chr4 | 7295984 | 7296413 | chr11 | 850376 | 850601 |
| chr19 | 3423146 | 3423776 | chr19 | 30401986 | 30403319 | chr16 | 35694585 | 35696650 |
| chr6 | 138795712 | 138796046 | chr2 | 128371968 | 128373110 | chr11 | 1554164 | 1555077 |
| chr19 | 2968830 | 2969704 | chr16 | 46406326 | 46407055 | chr19 | 38988875 | 38989411 |
| chr1 | 18598422 | 18599209 | chr9 | 136305767 | 136306003 | chr20 | 63405284 | 63405882 |
| chr13 | 113184875 | 113185558 | chr1 | 29685004 | 29686472 | chr19 | 57707994 | 57708427 |
| chr3 | 129554716 | 129555496 | chr15 | 69885753 | 69886484 | chr19 | 45104772 | 45105306 |
| chr20 | 14226365 | 14226836 | chr4 | 187088185 | 187089682 | chr12 | 8799699 | 8802313 |
| chr19 | 1945475 | 1946094 | chr1 | 21537714 | 21538964 | chr5 | 672242 | 672735 |
| chr8 | 142680963 | 142681240 | chr2 | 10843447 | 10844386 | chr1 | 3065915 | 3066519 |
| chr17 | 4772927 | 4773702 | chr12 | 5170681 | 5171792 | chr16 | 87637576 | 87638215 |
| chr19 | 5580164 | 5580692 | chr5 | 170922541 | 170922834 | chr6 | 150849425 | 150849844 |
| chr9 | 136669268 | 136669988 | chr8 | 28318334 | 28318857 | chr1 | 183215294 | 183215826 |
| chr11 | 2385577 | 2386469 | chr8 | 143220298 | 143221031 | chr8 | 141792723 | 141793407 |
| chr4 | 82042170 | 82043392 | chr3 | 195811909 | 195812659 | chr12 | 1820956 | 1821526 |
| chr14 | 44557203 | 44557742 | chr8 | 22882938 | 22884470 | chr9 | 96087158 | 96087591 |
| chr16 | 69482118 | 69482615 | chr16 | 48203215 | 48204314 | chr10 | 93180541 | 93180982 |
| chr17 | 49915787 | 49916148 | chr1 | 231159914 | 231160760 | chr13 | 111897196 | 111898006 |
| chr17 | 6162421 | 6163521 | chr2 | 94594615 | 94594893 | chr2 | 124894596 | 124896738 |
| chr11 | 71534903 | 71535562 | chr13 | 114100141 | 114100538 | chr3 | 38932392 | 38932715 |
| chr12 | 131379609 | 131380266 | chr9 | 133918204 | 133918542 | chr5 | 1174259 | 1174780 |
| chr6 | 45710050 | 45711193 | chr19 | 4019139 | 4020219 | chr16 | 64003748 | 64004006 |
| chr12 | 49543901 | 49544549 | chr16 | 27113069 | 27113633 | chr20 | 49424639 | 49425267 |
| chr19 | 13817304 | 13818393 | chr9 | 126639882 | 126641195 | chr5 | 10681023 | 10681845 |
| chr15 | 81324891 | 81325559 | chr4 | 44185960 | 44186740 | chr22 | 48003945 | 48004677 |
| chr5 | 3301184 | 3301800 | chr19 | 28920044 | 28920979 | chr7 | 94089210 | 94090442 |
| chr21 | 15289569 | 15290531 | chr3 | 45035760 | 45036675 | chr6 | 33173901 | 33174540 |
| chr10 | 21145725 | 21146256 | chr16 | 35146017 | 35146511 | chr6 | 25732632 | 25733056 |
| chr2 | 77088187 | 77090189 | chr2 | 206256029 | 206258082 | chr1 | 4001484 | 4002177 |
| chr18 | 79796822 | 79797388 | chr6 | 41469609 | 41469972 | chr1 | 29789347 | 29789945 |
| chr10 | 29142676 | 29143709 | chr12 | 124532346 | 124533295 | chr11 | 36727970 | 36728732 |
| chr1 | 37029186 | 37030303 | chr1 | 20257504 | 20258402 | chr7 | 56482485 | 56483225 |
| chr11 | 20607076 | 20610011 | chr5 | 146786375 | 146788563 | chr15 | 84953967 | 84955193 |
| chr4 | 140579449 | 140580635 | chr12 | 114052784 | 114053756 | chr5 | 141388907 | 141389315 |
| chr16 | 81785709 | 81786654 | chr17 | 47866980 | 47867741 | chr3 | 184597476 | 184598154 |
| chr17 | 3862442 | 3863587 | chr5 | 1447725 | 1448305 | chr9 | 14977816 | 14978251 |
| chr2 | 1137985 | 1138262 | chr8 | 40896676 | 40897321 | chr14 | 20867227 | 20869485 |
| chr10 | 124159453 | 124160506 | chr19 | 54591869 | 54592884 | chr16 | 88789177 | 88790967 |
| chr13 | 112346829 | 112348018 | chr21 | 10569261 | 10569764 | chr20 | 52975676 | 52978478 |
| chr2 | 26476391 | 26477336 | chr1 | 3313464 | 3314444 | chr18 | 37526296 | 37527037 |
| chr19 | 1304178 | 1304728 | chr10 | 80714702 | 80714947 | chr14 | 104820261 | 104820804 |
| chr12 | 124786500 | 124786918 | chr1 | 201647801 | 201649297 | chr12 | 8860999 | 8861782 |
| chr10 | 1292667 | 1292842 | chr10 | 129893661 | 129893999 | chr6 | 21523121 | 21523411 |
| chr18 | 61829764 | 61831458 | chr1 | 37146099 | 37148182 | chr11 | 61736975 | 61737307 |
| chr12 | 49962181 | 49962807 | chr6 | 149986298 | 149986662 | chr8 | 142417909 | 142418381 |
| chr9 | 135732986 | 135734665 | chr16 | 2476088 | 2476670 | chr10 | 133527885 | 133529561 |
| chr4 | 40513860 | 40514982 | chr6 | 170162545 | 170163300 | chr11 | 64800859 | 64801529 |
| chr5 | 174589638 | 174590093 | chr1 | 240055445 | 240058321 | chr6 | 24354901 | 24357546 |
| chr17 | 79081963 | 79082495 | chr12 | 3084154 | 3084888 | chr10 | 123446279 | 123446862 |
| chr5 | 1634944 | 1635029 | chr2 | 135797968 | 135798710 | chr11 | 132792787 | 132793256 |
| chr1 | 69312180 | 69312915 | chr3 | 127423301 | 127424186 | chr10 | 5479895 | 5480504 |
| chr4 | 90314972 | 90319427 | chr8 | 6153776 | 6155599 | chr12 | 125296786 | 125297409 |
| chr11 | 60019474 | 60020212 | chr11 | 33777906 | 33778511 | chr10 | 48604136 | 48604602 |
| chr9 | 126631201 | 126632164 | chr6 | 27675054 | 27675415 | chr16 | 11386791 | 11387487 |
| chr10 | 114179622 | 114180460 | chr2 | 27494264 | 27494748 | chr1 | 3668250 | 3669148 |
| chr13 | 46437813 | 46438288 | chr2 | 28379927 | 28380944 | chr4 | 3226115 | 3227164 |
| chr19 | 15457707 | 15458031 | chr8 | 21466988 | 21467516 | chr20 | 28602697 | 28603251 |
| chr14 | 103156755 | 103157430 | chr20 | 63417551 | 63418216 | chr12 | 34386309 | 34386838 |
| chr2 | 174729354 | 174729930 | chr20 | 39763007 | 39764238 | chr7 | 156150608 | 156151399 |
| chr2 | 72895199 | 72896215 | chr16 | 51440086 | 51440824 | chr17 | 45777015 | 45777980 |
| chr11 | 2381010 | 2381610 | chr19 | 48061779 | 48062144 | chr7 | 154484208 | 154486353 |
| chr12 | 96223508 | 96225171 | chr17 | 34628814 | 34629814 | chr5 | 174306416 | 174307447 |
| chr3 | 182740173 | 182741607 | chr7 | 136980865 | 136983981 | chr12 | 128191062 | 128191502 |
| chr9 | 126586423 | 126586869 | chr1 | 200247370 | 200247950 | chr12 | 6155117 | 6156291 |
| chr8 | 42497346 | 42498927 | chr22 | 16403707 | 16404446 | chr19 | 32082232 | 32084435 |
| chr9 | 35906104 | 35906477 | chr16 | 81615998 | 81616635 | chr6 | 27728339 | 27728509 |
| chr19 | 38564735 | 38564952 | chr15 | 23073357 | 23074002 | chr11 | 113246349 | 113247842 |
| chr9 | 131616128 | 131617374 | chr19 | 41911409 | 41912165 | chr2 | 118812801 | 118813802 |
| chr17 | 57473140 | 57474450 | chr17 | 28381140 | 28381902 | chr14 | 94956173 | 94957276 |
| chr7 | 156987193 | 156987498 | chr12 | 34363906 | 34364730 | chr22 | 22257034 | 22257727 |
| chr11 | 120523206 | 120523725 | chr1 | 223264477 | 223265238 | chr19 | 47424915 | 47425265 |
| chr17 | 62705232 | 62705840 | chr1 | 220895492 | 220895886 | chr7 | 1011722 | 1012378 |
| chr2 | 10831386 | 10832210 | chr6 | 29863338 | 29865418 | chr11 | 66412201 | 66412871 |
| chr19 | 51109327 | 51112112 | chr7 | 23490564 | 23492049 | chr1 | 29766860 | 29767418 |
| chr10 | 129768684 | 129770141 | chr16 | 87891874 | 87892326 | chr3 | 13147636 | 13148804 |
| chr9 | 126688168 | 126689442 | chr8 | 66178219 | 66179215 | chr14 | 98582007 | 98584529 |
| chr1 | 2523647 | 2524150 | chr19 | 39265609 | 39265850 | chr11 | 1558656 | 1559128 |
| chr9 | 95552439 | 95553265 | chr12 | 132836507 | 132837101 | chr14 | 104184632 | 104184981 |
| chr8 | 142219061 | 142219839 | chr4 | 75079164 | 75081428 | chr10 | 6432447 | 6432816 |
| chr2 | 60297801 | 60298377 | chr14 | 53735288 | 53737417 | chr20 | 38246754 | 38247230 |
| chr7 | 99972395 | 99973261 | chr22 | 39655346 | 39656537 | chr20 | 42487498 | 42489447 |
| chr17 | 17663381 | 17663778 | chr6 | 170103002 | 170104232 | chr2 | 85951675 | 85952628 |
| chr20 | 56708518 | 56709170 | chr11 | 115645498 | 115645915 | chr7 | 1226723 | 1226981 |
| chr18 | 79626015 | 79626513 | chr1 | 175479633 | 175482501 | chr19 | 19539794 | 19540371 |
| chr1 | 34625542 | 34627537 | chr3 | 139764012 | 139767447 | chr2 | 241858594 | 241859507 |
| chr12 | 132214831 | 132215310 | chr17 | 77671453 | 77673646 | chr8 | 42292790 | 42293646 |
| chr17 | 4003359 | 4003808 | chr4 | 6379635 | 6380754 | chr1 | 3188056 | 3188588 |
| chr5 | 176692724 | 176693137 | chr2 | 53004939 | 53005449 | chr7 | 156463606 | 156464174 |
| chr7 | 101751046 | 101751564 | chr19 | 1670509 | 1671456 | chr19 | 3386630 | 3387771 |
| chr22 | 37425549 | 37426544 | chr11 | 59062785 | 59063450 | chr3 | 194098759 | 194099611 |
| chr11 | 58066828 | 58067858 | chr22 | 45437452 | 45438091 | chr12 | 13203038 | 13204244 |
| chr15 | 80256001 | 80257546 | chr22 | 19174422 | 19175648 | chr1 | 145453912 | 145454196 |
| chr22 | 37179022 | 37179429 | chr8 | 143174231 | 143174475 | chr5 | 132271335 | 132272034 |
| chr17 | 47982343 | 47983100 | chr16 | 55502676 | 55503144 | chr7 | 135711167 | 135712319 |
| chr10 | 96412224 | 96413472 | chr18 | 78193334 | 78194091 | chr4 | 4667386 | 4667951 |
| chr18 | 79787094 | 79787550 | chr6 | 1670940 | 1671334 | chr11 | 131135789 | 131137070 |
| chr11 | 68370532 | 68370872 | chr7 | 101052947 | 101053495 | chr8 | 20293856 | 20294696 |
| chr9 | 134208401 | 134208700 | chr17 | 67246558 | 67247172 | chr1 | 5796416 | 5797200 |
| chr6 | 99352177 | 99354683 | chr3 | 197655792 | 197656247 | chr17 | 14211395 | 14212222 |
| chr5 | 60210976 | 60211228 | chr2 | 153442781 | 153443784 | chr8 | 138768109 | 138768704 |
| chr17 | 79398872 | 79399447 | chr6 | 169654368 | 169655364 | chr20 | 1388454 | 1391441 |
| chr16 | 67403811 | 67404048 | chr13 | 112442899 | 112443473 | chr5 | 68187578 | 68188015 |
| chr20 | 63322320 | 63323056 | chr6 | 30359193 | 30361418 | chr5 | 2537614 | 2538325 |
| chr12 | 52964673 | 52965243 | chr14 | 100549593 | 100550052 | chr1 | 56911394 | 56912552 |
| chr18 | 44696440 | 44698567 | chr3 | 57130626 | 57131063 | chr12 | 3132756 | 3133064 |
| chr17 | 42670551 | 42673054 | chr12 | 129456873 | 129459377 | chr15 | 27573998 | 27574447 |
| chr10 | 132389028 | 132389847 | chr19 | 33637989 | 33638426 | chr1 | 237205265 | 237206002 |
| chr8 | 48870989 | 48871745 | chr12 | 129150737 | 129151097 | chr19 | 4784547 | 4785016 |
| chr19 | 608732 | 609422 | chr6 | 32096862 | 32097179 | chr1 | 30804368 | 30805708 |
| chr2 | 27716717 | 27717365 | chr20 | 22490670 | 22491347 | chr22 | 36548710 | 36549924 |
| chr15 | 75786170 | 75786580 | chr16 | 4747107 | 4747928 | chr16 | 1859975 | 1863160 |
| chr12 | 104181488 | 104182781 | chr18 | 76208270 | 76209568 | chr7 | 27425598 | 27426185 |
| chr1 | 25918460 | 25919402 | chr12 | 132285404 | 132286001 | chr16 | 88539608 | 88540404 |
| chr19 | 2559584 | 2560223 | chr14 | 53084806 | 53085285 | chr5 | 147576050 | 147576658 |
| chr11 | 123235367 | 123236123 | chr1 | 18667995 | 18669638 | chr11 | 134008529 | 134009819 |
| chr16 | 27892903 | 27893414 | chr2 | 201660444 | 201661856 | chr14 | 105571418 | 105571614 |
| chr18 | 37488401 | 37490448 | chr11 | 45929701 | 45930409 | chr2 | 34569886 | 34571295 |
| chr17 | 3379617 | 3380526 | chr9 | 132414084 | 132415424 | chr10 | 111207791 | 111210483 |
| chr15 | 59813799 | 59817297 | chr10 | 119477761 | 119479985 | chr12 | 117686684 | 117687519 |
| chr1 | 68046961 | 68047381 | chr1 | 20718862 | 20719453 | chr2 | 207771138 | 207771336 |
| chr3 | 195606210 | 195609285 | chr10 | 124363776 | 124364611 | chr14 | 105905495 | 105906963 |
| chr15 | 47846016 | 47848477 | chr10 | 79006913 | 79007460 | chr10 | 130558359 | 130559284 |
| chr11 | 120832599 | 120832995 | chr5 | 3181267 | 3182040 | chr19 | 52997517 | 52997945 |
| chr11 | 2788239 | 2788848 | chr11 | 71358046 | 71358719 | chr6 | 160740308 | 160741446 |
| chr6 | 31116945 | 31117637 | chr16 | 11526780 | 11527379 | chr20 | 63954799 | 63955430 |
| chr20 | 61179062 | 61180643 | chr11 | 4175065 | 4176112 | chr5 | 175847108 | 175847460 |
| chr10 | 8033055 | 8033801 | chr20 | 38789914 | 38791512 | chr3 | 13553447 | 13554043 |
| chr1 | 38047814 | 38048219 | chr4 | 147508169 | 147509610 | chr19 | 5881145 | 5882166 |
| chr11 | 61008199 | 61008725 | chr19 | 32091152 | 32092240 | chr20 | 63382832 | 63383484 |
| chr15 | 60118364 | 60119357 | chr9 | 127550834 | 127551187 | chr12 | 130712448 | 130713641 |
| chr2 | 232525905 | 232526980 | chr6 | 27559708 | 27560513 | chr2 | 10431757 | 10432327 |
| chr1 | 226799248 | 226799586 | chr12 | 106194943 | 106196943 | chr19 | 22617533 | 22618238 |
| chr19 | 22831488 | 22831822 | chr12 | 130070048 | 130070665 | chr4 | 6428464 | 6429664 |
| chr12 | 119114091 | 119115188 | chr19 | 1318515 | 1319629 | chr9 | 136156483 | 136157344 |
| chr9 | 137230298 | 137230862 | chr4 | 187173813 | 187174219 | chr6 | 28696335 | 28698254 |
| chr10 | 121630774 | 121631920 | chr7 | 48606658 | 48607324 | chr17 | 6041211 | 6041533 |
| chr10 | 12466783 | 12467445 | chr13 | 98043712 | 98044607 | chr11 | 129390700 | 129391876 |
| chr1 | 156646792 | 156647001 | chr2 | 94207646 | 94208065 | chr12 | 113697741 | 113698443 |
| chr22 | 50237660 | 50237983 | chr3 | 129594180 | 129594755 | chr17 | 3176551 | 3176909 |
| chr11 | 124870611 | 124871544 | chr8 | 10726307 | 10726935 | chr5 | 15556992 | 15557237 |
| chr11 | 86799581 | 86799991 | chr19 | 54507831 | 54508723 | chr4 | 5893900 | 5894430 |
| chr17 | 19748417 | 19748861 | chr19 | 43316217 | 43317050 | chr13 | 111473849 | 111474453 |
| chr18 | 48778004 | 48779119 | chr9 | 135287726 | 135287996 | chr20 | 63932958 | 63933720 |
| chr20 | 36864736 | 36865042 | chr5 | 372821 | 373375 | chr22 | 44865381 | 44865794 |
| chr16 | 1106200 | 1107159 | chr19 | 3383313 | 3384200 | chr8 | 134430109 | 134431577 |
| chr11 | 44324483 | 44325524 | chr1 | 148013278 | 148013640 | chr14 | 102560543 | 102561316 |
| chr3 | 123431935 | 123432616 | chr20 | 31192550 | 31193325 | chr5 | 1170047 | 1170780 |
| chr10 | 106648934 | 106649403 | chr20 | 30377335 | 30377681 | chr2 | 545438 | 545930 |
| chr7 | 154583772 | 154584605 | chr11 | 69746972 | 69747170 | chr3 | 50193062 | 50193605 |
| chr8 | 23571090 | 23571434 | chr14 | 29805241 | 29808012 | chr16 | 52101604 | 52102084 |
| chr1 | 151101403 | 151102308 | chr7 | 58047936 | 58049629 | chr4 | 54094115 | 54094911 |
| chr5 | 175326611 | 175328401 | chr6 | 166305925 | 166306627 | chr20 | 57288812 | 57289243 |
| chr19 | 13281465 | 13282213 | chr8 | 56889286 | 56892040 | chr10 | 1710578 | 1711112 |
| chr22 | 37500840 | 37502056 | chr4 | 9977982 | 9979494 | chr2 | 134550795 | 134551453 |
| chr4 | 164977224 | 164977983 | chr7 | 42685819 | 42686799 | chr6 | 31180448 | 31181128 |
| chr8 | 143891341 | 143891677 | chr10 | 130732304 | 130733974 | chr8 | 3104425 | 3105287 |
| chr6 | 30458539 | 30461213 | chr2 | 3785363 | 3785851 | chr10 | 125370740 | 125371278 |
| chr11 | 44326965 | 44329704 | chr13 | 29510491 | 29510879 | chr12 | 130705316 | 130705928 |
| chr1 | 87201929 | 87202821 | chr7 | 55004371 | 55006590 | chr6 | 1536792 | 1537650 |
| chr10 | 108598773 | 108599687 | chr21 | 41684959 | 41685104 | chr14 | 92548665 | 92549846 |
| chr11 | 126203282 | 126203711 | chr5 | 38256151 | 38256875 | chr2 | 96955194 | 96956062 |
| chr13 | 83877547 | 83878893 | chr7 | 1087423 | 1088830 | chr2 | 5802980 | 5804367 |
| chr10 | 15751395 | 15752577 | chr7 | 155349481 | 155349948 | chr12 | 106578527 | 106580196 |
| chr8 | 143048057 | 143048230 | chr17 | 49975633 | 49976684 | chr15 | 52242079 | 52243614 |
| chr18 | 75578 | 75989 | chr15 | 26841784 | 26843192 | chr17 | 74920237 | 74921163 |
| chr20 | 23047336 | 23047810 | chr16 | 17555542 | 17556727 | chr19 | 1773369 | 1774281 |
| chr12 | 54058093 | 54059930 | chr7 | 57203947 | 57205538 | chr18 | 37322563 | 37323099 |
| chr2 | 236803568 | 236804168 | chr7 | 3879134 | 3880403 | chr8 | 96038145 | 96039081 |
| chr19 | 50826911 | 50827126 | chr16 | 31329589 | 31330320 | chr14 | 99017458 | 99018008 |
| chr20 | 56249434 | 56249871 | chr5 | 169945468 | 169945993 | chr21 | 45615219 | 45615563 |
| chr2 | 187461437 | 187461848 | chr12 | 1920982 | 1921385 | chr6 | 150024439 | 150025275 |
| chr3 | 184526024 | 184526594 | chr17 | 76806327 | 76806881 | chr6 | 108233566 | 108234492 |
| chr18 | 6109474 | 6111607 | chr8 | 11062816 | 11063993 | chr5 | 1925377 | 1925723 |
| chr9 | 134390517 | 134391030 | chr17 | 32283785 | 32284371 | chr18 | 78914453 | 78914898 |
| chr19 | 55513512 | 55515132 | chr3 | 129046313 | 129046614 | chr1 | 43007273 | 43007503 |
| chr7 | 29289096 | 29290491 | chr11 | 127345598 | 127346361 | chr2 | 71966182 | 71967976 |
| chr11 | 64530226 | 64530654 | chr16 | 24686733 | 24688666 | chr5 | 112141159 | 112144569 |
| chr1 | 184223047 | 184223751 | chr15 | 26874890 | 26876791 | chr1 | 165934455 | 165935234 |
| chr17 | 81048998 | 81049801 | chr16 | 3575934 | 3576513 | chr8 | 41607277 | 41608375 |
| chr5 | 51668 | 53048 | chr22 | 27032270 | 27032836 | chr22 | 36587683 | 36588069 |
| chr20 | 39068634 | 39070796 | chr2 | 2111286 | 2111894 | chr14 | 60524569 | 60527747 |
| chr4 | 2401285 | 2402063 | chr10 | 125283962 | 125284735 | chr12 | 125430102 | 125430431 |
| chr15 | 100877335 | 100878282 | chr1 | 57728126 | 57728556 | chr22 | 43396995 | 43397234 |
| chr2 | 47728769 | 47730317 | chr7 | 117249663 | 117251975 | chr3 | 125882399 | 125883168 |
| chr7 | 94656272 | 94657031 | chr1 | 19645563 | 19646608 | chr17 | 48310021 | 48311196 |
| chr17 | 45791646 | 45792311 | chr10 | 121580269 | 121580798 | chr1 | 18199419 | 18200139 |
| chr6 | 27547261 | 27547552 | chr17 | 77555806 | 77556417 | chr15 | 27990349 | 27990831 |
| chr2 | 217789665 | 217790555 | chr16 | 86960605 | 86961135 | chr7 | 2304523 | 2305268 |
| chr5 | 1593395 | 1594306 | chr9 | 2883289 | 2886364 | chr15 | 100310132 | 100310778 |
| chr10 | 117511909 | 117512684 | chr22 | 27713117 | 27714319 | chr13 | 25678073 | 25680308 |
| chr11 | 2807098 | 2807653 | chr4 | 8276657 | 8277310 | chr8 | 11496588 | 11497421 |
| chr5 | 179851963 | 179853346 | chr17 | 50621006 | 50621615 | chr12 | 128778288 | 128779397 |
| chr4 | 7226434 | 7226838 | chr12 | 57624879 | 57625713 | chr9 | 130523569 | 130524228 |
| chr13 | 20390495 | 20390962 | chr1 | 242936772 | 242937453 | chr20 | 61816643 | 61817119 |
| chr16 | 72921447 | 72922069 | chr1 | 4664572 | 4665382 | chr20 | 51077240 | 51078004 |
| chr13 | 111900354 | 111900941 | chr16 | 57036702 | 57037704 | chr11 | 1890546 | 1891240 |
| chr1 | 3357069 | 3357699 | chr16 | 80491859 | 80493518 | chr12 | 117669074 | 117670851 |
| chr16 | 273801 | 274304 | chr5 | 179417046 | 179417842 | chr10 | 131085107 | 131085553 |
| chr3 | 138979330 | 138980378 | chr6 | 43865324 | 43866143 | chr10 | 132309014 | 132309576 |
| chr17 | 10123801 | 10124302 | chr19 | 38596093 | 38596711 | chr7 | 100785450 | 100786047 |
| chr6 | 16228902 | 16229874 | chr18 | 37293704 | 37295234 | chr19 | 19213450 | 19214091 |
| chr10 | 50993061 | 50993689 | chr9 | 108002439 | 108002975 | chr11 | 704785 | 705748 |
| chr10 | 127176101 | 127176463 | chr7 | 4928053 | 4928512 | chr3 | 14549105 | 14550381 |
| chr1 | 36920130 | 36921442 | chr6 | 28802015 | 28804118 | chr10 | 38498503 | 38501326 |
| chr5 | 141199793 | 141200245 | chr4 | 6009325 | 6009789 | chr8 | 101223885 | 101224544 |
| chr8 | 41249782 | 41250236 | chr1 | 18072222 | 18072937 | chr21 | 15619516 | 15621104 |
| chr21 | 44375156 | 44375577 | chr10 | 5851472 | 5852784 | chr8 | 34962545 | 34963454 |
| chr7 | 44293425 | 44294411 | chr2 | 218998442 | 218998909 | chr3 | 52781814 | 52782134 |
| chr17 | 185433 | 186118 | chr9 | 127795931 | 127796487 | chr12 | 52116052 | 52116565 |
| chr4 | 7298454 | 7299427 | chr10 | 101047044 | 101047715 | chr12 | 52346940 | 52347352 |
| chr19 | 6669960 | 6670843 | chr16 | 78246730 | 78247345 | chr2 | 129048041 | 129051355 |
| chr9 | 134598417 | 134598841 | chr11 | 5807634 | 5809525 | chr5 | 5304076 | 5304605 |
| chr6 | 33855078 | 33855633 | chr9 | 138022499 | 138023191 | chr20 | 42843067 | 42844829 |
| chr10 | 8071694 | 8072452 | chr9 | 137315565 | 137315861 | chr12 | 34599355 | 34600275 |
| chr14 | 28264222 | 28265000 | chr18 | 58402107 | 58402813 | chr14 | 101312664 | 101313803 |
| chr19 | 35513731 | 35514506 | chr1 | 20490903 | 20491523 | chr16 | 6482585 | 6482892 |
| chr16 | 46572501 | 46573970 | chr17 | 28986169 | 28986530 | chr12 | 34376417 | 34376986 |
| chr5 | 162279359 | 162280759 | chr8 | 10672196 | 10673415 | chr9 | 136053185 | 136053756 |
| chr20 | 62561133 | 62561990 | chr21 | 40089425 | 40090931 | chr1 | 71046474 | 71046855 |
| chr4 | 134327093 | 134327599 | chr20 | 21693852 | 21695033 | chr19 | 47004862 | 47005417 |
| chr20 | 62710169 | 62710630 | chr2 | 133943398 | 133946619 | chr15 | 73745111 | 73746412 |
| chr12 | 34292808 | 34293258 | chr17 | 9842449 | 9843334 | chr10 | 42752389 | 42752820 |
| chr16 | 75535232 | 75535781 | chr15 | 83306007 | 83307128 | chr14 | 88321779 | 88322090 |
| chr19 | 55493807 | 55494647 | chr13 | 113022738 | 113023263 | chr10 | 131979938 | 131980291 |
| chr9 | 66265437 | 66266502 | chr16 | 6941994 | 6943062 | chr7 | 5864799 | 5865269 |
| chr11 | 126156004 | 126156215 | chr15 | 79451319 | 79451852 | chr7 | 139987872 | 139988610 |
| chr20 | 59526044 | 59526541 | chr15 | 98126697 | 98128739 | chr20 | 61051919 | 61052583 |
| chr2 | 74908379 | 74909208 | chr5 | 2591000 | 2591409 | chr9 | 8733588 | 8735676 |
| chr7 | 47536729 | 47537065 | chr1 | 234005351 | 234008059 | chr2 | 90237901 | 90238169 |
| chr11 | 61488462 | 61489326 | chr17 | 57607237 | 57607853 | chr17 | 391116 | 391742 |
| chr2 | 237496770 | 237497524 | chr7 | 127498 | 129616 | chr9 | 135416159 | 135417109 |
| chr20 | 58064352 | 58065362 | chr4 | 8418631 | 8419806 | chr18 | 11972404 | 11972803 |
| chr11 | 257694 | 258810 | chr1 | 29344262 | 29344802 | chr6 | 149931769 | 149933307 |
| chr19 | 46405279 | 46405665 | chr15 | 100863596 | 100864134 | chr2 | 70645280 | 70646318 |
| chr5 | 30345524 | 30346079 | chr4 | 3725598 | 3727115 | chr2 | 120487110 | 120488197 |
| chr16 | 70700289 | 70700828 | chr8 | 52099870 | 52100077 | chr12 | 1907998 | 1908617 |
| chr1 | 154418306 | 154421199 | chr19 | 8655477 | 8655931 | chr22 | 41779096 | 41780334 |
| chr11 | 63952213 | 63952537 | chr21 | 10653607 | 10654042 | chr9 | 133272137 | 133273348 |
| chr2 | 121281939 | 121283023 | chr20 | 37422967 | 37423702 | chr17 | 5797289 | 5797898 |
| chr14 | 95039144 | 95039665 | chr6 | 167373104 | 167373476 | chr4 | 32716127 | 32717286 |
| chr7 | 2979284 | 2979893 | chr17 | 79697965 | 79698648 | chr1 | 38427400 | 38428355 |
| chr20 | 51570578 | 51571042 | chr20 | 62469860 | 62470315 | chr2 | 2932769 | 2933583 |
| chr13 | 60900178 | 60901065 | chr1 | 84387543 | 84389396 | chr19 | 51054287 | 51055830 |
| chr17 | 39110507 | 39110882 | chr5 | 141414925 | 141415577 | chr12 | 39669435 | 39669928 |
| chr7 | 6528354 | 6529047 | chr3 | 55188316 | 55189111 | chr22 | 20670726 | 20671152 |
| chr6 | 27557115 | 27557566 | chr11 | 76252618 | 76253486 | chr16 | 86240374 | 86241364 |
| chr11 | 2811809 | 2812154 | chr5 | 1873281 | 1873882 | chr16 | 6704054 | 6707761 |
| chr14 | 55439218 | 55440405 | chr1 | 18111646 | 18112439 | chr1 | 176875247 | 176877472 |
| chr19 | 2675053 | 2675423 | chr4 | 7430471 | 7431003 | chr1 | 22872706 | 22873271 |
| chr1 | 3707337 | 3707862 | chr14 | 99571116 | 99571749 | chr4 | 5924319 | 5925671 |
| chr13 | 21912928 | 21913599 | chr8 | 98012351 | 98012769 | chr8 | 100165217 | 100165545 |
| chr1 | 118884864 | 118886940 | chr17 | 20895995 | 20896516 | chr19 | 3708664 | 3709375 |
| chr22 | 47090944 | 47091864 | chr10 | 128740419 | 128741877 | chr2 | 15038509 | 15040172 |
| chr17 | 74955275 | 74956382 | chr20 | 61278481 | 61279349 | chr2 | 862205 | 862450 |
| chr5 | 152072410 | 152073647 | chr20 | 3164907 | 3165410 | chr7 | 129203916 | 129204625 |
| chr1 | 201945986 | 201946319 | chr2 | 237012085 | 237012746 | chr1 | 175193227 | 175193626 |
| chr15 | 53830666 | 53832024 | chr17 | 66976103 | 66977141 | chr17 | 79693879 | 79694370 |
| chr20 | 43377711 | 43379852 | chr12 | 2980470 | 2981143 | chr15 | 40437499 | 40437962 |
| chr7 | 158386721 | 158387206 | chr18 | 79383821 | 79385291 | chr22 | 45893242 | 45893868 |
| chr2 | 217150735 | 217152231 | chr8 | 143039147 | 143039476 | chr7 | 48046464 | 48046736 |
| chr18 | 70234510 | 70235361 | chr20 | 21717151 | 21718536 | chr9 | 129868505 | 129868768 |
| chr5 | 6823069 | 6823587 | chr16 | 543466 | 543877 | chr1 | 7705891 | 7706440 |
| chr19 | 22517345 | 22518283 | chr5 | 560901 | 561045 | chr11 | 69611783 | 69612191 |
| chr8 | 48466156 | 48466691 | chr8 | 57280325 | 57280968 | chr11 | 379227 | 379657 |
| chr1 | 14773211 | 14773797 | chr12 | 130286087 | 130287000 | chr3 | 153362337 | 153363923 |
| chr10 | 86244295 | 86244573 | chr1 | 247518098 | 247518769 | chr4 | 8133146 | 8133644 |
| chr19 | 50874525 | 50875225 | chr12 | 131681808 | 131682228 | chr22 | 27271007 | 27271566 |
| chr7 | 62395540 | 62398139 | chr2 | 23558918 | 23559816 | chr4 | 153206815 | 153207404 |
| chr9 | 133653324 | 133654652 | chr1 | 245516101 | 245516905 | chr1 | 114052560 | 114054568 |
| chr21 | 37531031 | 37531869 | chr18 | 78825067 | 78825667 | chr9 | 133701475 | 133702217 |
| chr6 | 29795896 | 29798355 | chr21 | 45634490 | 45634950 | chr6 | 168782252 | 168782641 |
| chr15 | 88912315 | 88912686 | chr22 | 49286044 | 49286433 | chr12 | 51821631 | 51822184 |
| chr5 | 140829238 | 140829606 | chr1 | 236332509 | 236332975 | chr17 | 81453119 | 81453410 |
| chr10 | 121616821 | 121618122 | chr19 | 47545241 | 47545878 | chr4 | 3827917 | 3828399 |
| chr5 | 170607115 | 170607893 | chr1 | 153609351 | 153610065 | chr3 | 125065991 | 125066622 |
| chr10 | 75043631 | 75044207 | chr18 | 73320 | 74092 | chr4 | 159414546 | 159415220 |
| chr12 | 131602316 | 131602983 | chr16 | 361803 | 362780 | chr2 | 174748152 | 174748652 |
| chr17 | 45863453 | 45864357 | chr10 | 122818748 | 122818927 | chr17 | 5770042 | 5770958 |
| chr3 | 14297691 | 14298222 | chr20 | 46012306 | 46013456 | chr2 | 11446537 | 11448552 |
| chr12 | 34383970 | 34384209 | chr22 | 16262442 | 16262889 | chr2 | 241198453 | 241198942 |
| chr14 | 16114451 | 16116730 | chr19 | 40958660 | 40962203 | chr6 | 34002422 | 34003355 |
| chr10 | 133388795 | 133389435 | chr22 | 48428595 | 48429300 | chr15 | 66936826 | 66937324 |
| chr19 | 8685878 | 8686673 | chr11 | 2387580 | 2388089 | chr16 | 2040574 | 2040780 |
| chr3 | 11911248 | 11911518 | chr9 | 135742936 | 135743256 | chr14 | 105764455 | 105764805 |
| chr22 | 44358071 | 44358245 | chr20 | 62455050 | 62455371 | chr12 | 128149193 | 128149783 |
| chr20 | 59765618 | 59767355 | chr13 | 113509672 | 113510652 | chr17 | 45690667 | 45691361 |
| chr9 | 132514579 | 132515078 | chr1 | 2459283 | 2459968 | chr6 | 45799306 | 45802177 |
| chr17 | 83098992 | 83099681 | chr20 | 62847493 | 62847977 | chr4 | 7445010 | 7446568 |
| chr19 | 1465471 | 1466273 | chr6 | 34087981 | 34088374 | chr4 | 7084164 | 7084842 |
| chr12 | 113669547 | 113669977 | chr12 | 34352527 | 34352981 | chr22 | 45931159 | 45931977 |
| chr20 | 62964809 | 62965437 | chr18 | 5455738 | 5457802 | chr9 | 98458290 | 98458654 |
| chr17 | 2075789 | 2076307 | chr19 | 6287614 | 6291945 | chr11 | 78551577 | 78552962 |
| chr7 | 988240 | 988741 | chr18 | 10659439 | 10661230 | chr20 | 30317095 | 30317512 |
| chr5 | 173435568 | 173436056 | chr17 | 11244987 | 11245475 | chr11 | 55872646 | 55873456 |
| chr1 | 18008469 | 18009327 | chr16 | 87407956 | 87408084 | chr2 | 172329316 | 172330234 |
| chr17 | 72622903 | 72624053 | chr22 | 48544403 | 48545113 | chr19 | 38222223 | 38223024 |
| chr22 | 39663687 | 39664321 | chr22 | 39624316 | 39624688 | chr5 | 179504479 | 179505227 |
| chr7 | 1255496 | 1256232 | chr14 | 100165380 | 100165586 | chr2 | 18825249 | 18827179 |
| chr17 | 7740232 | 7740978 | chr5 | 180343007 | 180343495 | chr4 | 6367279 | 6368108 |
| chr22 | 44729515 | 44730336 | chr11 | 62390392 | 62391557 | chr7 | 129522650 | 129523190 |
| chr5 | 176701076 | 176701421 | chr13 | 37934514 | 37934890 | chr7 | 157613657 | 157614294 |
| chr14 | 20626152 | 20627657 | chr5 | 69015924 | 69018134 | chr2 | 71865504 | 71866253 |
| chr1 | 200356568 | 200359153 | chr20 | 63238149 | 63238794 | chr2 | 129587294 | 129587833 |
| chr12 | 4978891 | 4980342 | chr9 | 112947698 | 112948819 | chr2 | 731453 | 731871 |
| chr11 | 1453511 | 1453875 | chr17 | 80498155 | 80498703 | chr22 | 44714812 | 44715446 |
| chr16 | 73354506 | 73355148 | chr1 | 153626908 | 153628497 | chr15 | 26893591 | 26893943 |
| chr16 | 25761546 | 25763054 | chr2 | 1637397 | 1638209 | chr16 | 29184549 | 29185271 |
| chr1 | 18702999 | 18703337 | chr10 | 1400029 | 1400317 | chr17 | 19724423 | 19724935 |
| chr16 | 13733734 | 13736545 | chr2 | 175709232 | 175709878 | chr12 | 80249828 | 80250185 |
| chr15 | 96407943 | 96408462 | chr7 | 157961746 | 157962074 | chr5 | 170745903 | 170746637 |
| chr19 | 3321355 | 3321948 | chr5 | 173851909 | 173852476 | chr9 | 130473594 | 130473910 |
| chr5 | 1923167 | 1923963 | chr4 | 4224271 | 4225907 | chr13 | 43404975 | 43407206 |
| chr8 | 142620044 | 142620371 | chr10 | 132336615 | 132337650 | chr6 | 14709311 | 14709835 |
| chr12 | 45034914 | 45035185 | chr19 | 10032270 | 10033265 | chr15 | 68930617 | 68931041 |
| chr20 | 48456064 | 48456542 | chr12 | 119018390 | 119020547 | chr9 | 128910173 | 128910716 |
| chr3 | 3781489 | 3782644 | chr5 | 2847799 | 2849012 | chr9 | 135341248 | 135341663 |
| chr17 | 48740365 | 48741195 | chr7 | 158470313 | 158470889 | chr16 | 10818074 | 10818596 |
| chr10 | 117806867 | 117810859 | chr13 | 51170842 | 51172180 | chr10 | 100879014 | 100880145 |
| chr8 | 19602462 | 19602872 | chr9 | 21961202 | 21964439 | chr15 | 74296352 | 74296809 |
| chr17 | 58279068 | 58279472 | chr16 | 46414694 | 46415650 | chr2 | 1476995 | 1477267 |
| chr10 | 33348315 | 33349927 | chr19 | 50870568 | 50870874 | chr3 | 10443352 | 10443681 |
| chr12 | 124655621 | 124655872 | chr19 | 13459486 | 13459956 | chr15 | 99007529 | 99008299 |
| chr7 | 157876596 | 157876866 | chr22 | 48278153 | 48278878 | chr20 | 46268201 | 46268440 |
| chr12 | 106900712 | 106901921 | chr1 | 109711736 | 109712355 | chr4 | 7902210 | 7903022 |
| chr15 | 89030465 | 89031355 | chr17 | 79528919 | 79529949 | chr10 | 130215384 | 130216017 |
| chr12 | 131481794 | 131482598 | chr16 | 55812941 | 55814800 | chr2 | 30404128 | 30404841 |
| chr22 | 50301415 | 50302158 | chr10 | 43350924 | 43351358 | chr7 | 69369850 | 69371284 |
| chr1 | 19369075 | 19370020 | chr16 | 1245076 | 1245431 | chr13 | 22496483 | 22497394 |
| chr16 | 73889078 | 73890524 | chr17 | 27038242 | 27043585 | chr5 | 170706855 | 170710015 |
| chr12 | 34365887 | 34366402 | chr9 | 135069760 | 135070284 | chr1 | 202646632 | 202647780 |
| chr7 | 156516037 | 156517125 | chr19 | 55717664 | 55718310 | chr1 | 4461912 | 4462617 |
| chr5 | 179462092 | 179462706 | chr6 | 35292342 | 35292754 | chr9 | 135257320 | 135257679 |
| chr22 | 17037401 | 17037672 | chr5 | 180365680 | 180366511 | chr13 | 112941953 | 112942191 |
| chr19 | 46820783 | 46821357 | chr8 | 7054396 | 7055841 | chr6 | 1317434 | 1317699 |
| chr19 | 55463417 | 55464182 | chr16 | 86865675 | 86866289 | chr14 | 104509714 | 104510266 |
| chr7 | 75967878 | 75968255 | chr2 | 130966326 | 130966926 | chr17 | 58277881 | 58278293 |
| chr2 | 238985692 | 238986709 | chr7 | 58003779 | 58008277 | chr20 | 30537476 | 30537689 |
| chr7 | 156613034 | 156613540 | chr16 | 85436871 | 85437138 | chr13 | 60267671 | 60268104 |
| chr12 | 119156438 | 119156659 | chr1 | 43913156 | 43913875 | chr5 | 136079569 | 136081037 |
| chr8 | 866998 | 867866 | chr7 | 74023125 | 74023670 | chr18 | 74282484 | 74283445 |
| chr18 | 76406056 | 76406488 | chr2 | 219765196 | 219767640 | chr10 | 133396462 | 133397957 |
| chr5 | 112596716 | 112599068 | chr8 | 46131507 | 46132158 | chr15 | 70489435 | 70490566 |
| chr11 | 71598289 | 71598779 | chr7 | 157405631 | 157406085 | chr10 | 83912617 | 83913125 |
| chr12 | 117778304 | 117779718 | chr1 | 25503152 | 25503942 | chr22 | 42645385 | 42645743 |
| chr6 | 165984816 | 165985244 | chr1 | 22167331 | 22168159 | chr19 | 22832492 | 22832682 |
| chr4 | 103571184 | 103572864 | chr2 | 108921299 | 108921902 | chr2 | 227387534 | 227388093 |
| chr22 | 47395282 | 47395993 | chr20 | 19108479 | 19110990 | chr7 | 155926275 | 155926991 |
| chr9 | 121607895 | 121608236 | chr11 | 10354101 | 10356081 | chr20 | 63767921 | 63768533 |
| chr4 | 6008123 | 6008496 | chr1 | 203150733 | 203151619 | chr1 | 36723154 | 36723954 |
| chr10 | 130035287 | 130036625 | chr12 | 132602400 | 132602947 | chr19 | 852747 | 854215 |
| chr12 | 50072465 | 50073518 | chr12 | 128323576 | 128324144 | chr2 | 1083378 | 1083941 |
| chr2 | 1397711 | 1398704 | chr10 | 74728398 | 74731159 | chr7 | 104735212 | 104736497 |
| chr4 | 183097214 | 183097588 | chr17 | 79678581 | 79679200 | chr1 | 205849728 | 205850472 |
| chr10 | 80917675 | 80920207 | chr18 | 76658818 | 76659391 | chr17 | 334227 | 335083 |
| chr17 | 81421724 | 81422124 | chr11 | 66406519 | 66408064 | chr11 | 61758746 | 61759468 |
| chr14 | 76137790 | 76138270 | chr14 | 21777620 | 21778789 | chr12 | 107379471 | 107380613 |
| chr6 | 170484649 | 170485542 | chr13 | 26742572 | 26742981 | chr13 | 21037593 | 21038169 |
| chr5 | 2199735 | 2200073 | chr16 | 77380937 | 77381586 | chr12 | 119144572 | 119145523 |
| chr2 | 126894886 | 126895681 | chr14 | 93938891 | 93940273 | chr5 | 6595559 | 6596372 |
| chr5 | 3609047 | 3609373 | chr21 | 33867758 | 33868305 | chr3 | 140120035 | 140121306 |
| chr19 | 33619351 | 33619946 | chr21 | 10688116 | 10688476 | chr22 | 20155326 | 20155623 |
| chr7 | 56483874 | 56484516 | chr20 | 990676 | 991150 | chr1 | 214488646 | 214490166 |
| chr19 | 21883688 | 21884681 | chr12 | 132219019 | 132220119 | chr17 | 18383007 | 18383483 |
| chr17 | 43418946 | 43419322 | chr11 | 48899964 | 48900268 | chr19 | 5229160 | 5229499 |
| chr20 | 35456044 | 35456574 | chr10 | 18710217 | 18710580 | chr10 | 120418217 | 120420444 |
| chr19 | 7783704 | 7784301 | chr22 | 23511451 | 23512289 | chr18 | 46588752 | 46590698 |
| chr14 | 76113463 | 76114085 | chr2 | 117858645 | 117859456 | chr3 | 32977459 | 32978285 |
| chr5 | 13810103 | 13810224 | chr1 | 80632428 | 80634135 | chr6 | 28765071 | 28765318 |
| chr19 | 54289075 | 54290441 | chr5 | 180331316 | 180331965 | chr4 | 3813264 | 3813568 |
| chr12 | 54041745 | 54043182 | chr20 | 44750094 | 44750791 | chr1 | 21594354 | 21594867 |
| chr17 | 80526162 | 80526728 | chr13 | 111410851 | 111411393 | chr2 | 23575920 | 23576534 |
| chr5 | 539686 | 540304 | chr20 | 49635874 | 49636157 | chr16 | 8179018 | 8180259 |
| chr1 | 22593072 | 22593713 | chr15 | 65361878 | 65363020 | chr16 | 54208872 | 54209263 |
| chr11 | 61026157 | 61026819 | chr15 | 66626423 | 66627762 | chr1 | 31448397 | 31450636 |
| chr3 | 128006326 | 128006955 | chr13 | 22736345 | 22737013 | chr1 | 10863046 | 10863608 |
| chr8 | 6550185 | 6550396 | chr10 | 123428377 | 123428670 | chr20 | 30895369 | 30895807 |
| chr1 | 104073551 | 104073891 | chr2 | 224435437 | 224436699 | chr17 | 48513166 | 48514171 |
| chr17 | 5878024 | 5878520 | chr9 | 131604069 | 131604865 | chr4 | 6370328 | 6371134 |
| chr2 | 234383883 | 234385348 | chr10 | 117428470 | 117428704 | chr12 | 34382267 | 34382590 |
| chr11 | 64298111 | 64298616 | chr17 | 39057497 | 39058002 | chr16 | 86710715 | 86711209 |
| chr2 | 238098825 | 238099836 | chr17 | 77037137 | 77037644 | chr2 | 236162242 | 236162821 |
| chr19 | 4758577 | 4759442 | chr4 | 3701741 | 3702244 | chr2 | 170310189 | 170312162 |
| chr2 | 100284230 | 100285872 | chr10 | 26219636 | 26221859 | chr16 | 1010275 | 1010593 |
| chr4 | 166501271 | 166502208 | chr2 | 122307825 | 122308403 | chr6 | 169939288 | 169939680 |
| chr18 | 51284691 | 51285938 | chr7 | 28662281 | 28662534 | chr16 | 21717541 | 21718178 |
| chr4 | 55169732 | 55170855 | chr12 | 38152867 | 38153361 | chr7 | 1429866 | 1430391 |
| chr1 | 157962637 | 157963597 | chr15 | 65952431 | 65953166 | chr6 | 39159248 | 39159589 |
| chr14 | 89370465 | 89371352 | chr9 | 130323479 | 130324236 | chr11 | 43992756 | 43993641 |
| chr8 | 20301936 | 20302365 | chr7 | 73596710 | 73597709 | chr4 | 7306436 | 7306988 |
| chr11 | 90052743 | 90053535 | chr14 | 23671889 | 23674341 | chr2 | 149320491 | 149321297 |
| chr5 | 150217743 | 150218967 | chr13 | 24989264 | 24989419 | chr5 | 180207790 | 180208298 |
| chr15 | 86905980 | 86908786 | chr19 | 19209353 | 19209870 | chr20 | 30899653 | 30900310 |
| chr10 | 100567179 | 100568156 | chr22 | 46613943 | 46614773 | chr5 | 16785125 | 16785742 |
| chr11 | 116232614 | 116233592 | chr16 | 1433028 | 1433498 | chr3 | 66655379 | 66656077 |
| chr4 | 174180826 | 174181021 | chr16 | 87621504 | 87622234 | chr2 | 235206247 | 235206647 |
| chr10 | 131517427 | 131517881 | chr21 | 35064954 | 35065442 | chr4 | 121932400 | 121932740 |
| chr1 | 4310091 | 4310590 | chr2 | 98295136 | 98296099 | chr1 | 234231547 | 234231851 |
| chr7 | 156604370 | 156604553 | chr19 | 3290011 | 3290390 | chr20 | 62959041 | 62959858 |
| chr6 | 170423674 | 170424344 | chr10 | 79595318 | 79597851 | chr16 | 1069386 | 1069964 |
| chr1 | 229758175 | 229758663 | chr20 | 29742996 | 29743306 | chr16 | 63617278 | 63617758 |
| chr17 | 6654881 | 6655533 | chr1 | 37242395 | 37243347 | chr10 | 132211220 | 132212019 |
| chr16 | 88638897 | 88639981 | chr4 | 5918324 | 5919209 | chr19 | 2246397 | 2246831 |
| chr11 | 111221809 | 111222977 | chr13 | 95328795 | 95330554 | chr1 | 212923472 | 212924480 |
| chr2 | 91690 | 92927 | chr9 | 63814849 | 63815571 | chr2 | 230869641 | 230870028 |
| chr14 | 104851779 | 104852053 | chr21 | 42746263 | 42746995 | chr7 | 64942802 | 64943219 |
| chr7 | 58055639 | 58056141 | chr2 | 14535135 | 14536465 | chr11 | 70006866 | 70007522 |
| chr1 | 1034365 | 1034914 | chr21 | 14643819 | 14644485 | chr19 | 50167371 | 50167722 |
| chr6 | 15296745 | 15299739 | chr19 | 54255289 | 54255921 | chr9 | 27297374 | 27297807 |
| chr9 | 135100288 | 135100713 | chr5 | 28927511 | 28928628 | chr1 | 18551822 | 18552588 |
| chr16 | 7765182 | 7766676 | chr8 | 138129295 | 138129727 | chr9 | 136250736 | 136251010 |
| chr1 | 75124847 | 75125235 | chr7 | 72706649 | 72708783 | chr1 | 200870839 | 200871822 |
| chr9 | 123372686 | 123373333 | chr10 | 17866564 | 17867893 | chr1 | 201650366 | 201650593 |
| chr3 | 52067783 | 52068636 | chr9 | 134488394 | 134488813 | chr5 | 178291195 | 178291709 |
| chr19 | 55067325 | 55068601 | chr19 | 29875616 | 29876254 | chr22 | 41424909 | 41426921 |
| chr19 | 17807255 | 17808364 | chr8 | 2727396 | 2728551 | chr10 | 133089028 | 133089343 |
| chr10 | 44008530 | 44009577 | chr15 | 70499090 | 70499764 | chr10 | 118572415 | 118573498 |
| chr10 | 6626009 | 6627116 | chr10 | 44863864 | 44864338 | chr17 | 21075751 | 21076092 |
| chr22 | 10728886 | 10729722 | chr21 | 10663705 | 10664321 | chr10 | 50660076 | 50660439 |
| chr5 | 76956393 | 76957107 | chr7 | 158588596 | 158589011 | chr16 | 54358281 | 54358652 |
| chr6 | 42329376 | 42329938 | chr19 | 29507406 | 29507724 | chr3 | 127363261 | 127363837 |
| chr8 | 2199399 | 2200120 | chr11 | 2299279 | 2299633 | chr14 | 77218733 | 77219644 |
| chr16 | 57260394 | 57260745 | chr13 | 35759734 | 35761161 | chr2 | 45170770 | 45171091 |
| chr20 | 29752520 | 29753637 | chr14 | 104980248 | 104980545 | chr9 | 133932305 | 133932761 |
| chr16 | 79315930 | 79316934 | chr2 | 121197493 | 121198068 | chr6 | 35516394 | 35517405 |
| chr5 | 80215734 | 80217586 | chr5 | 140796082 | 140796259 | chr6 | 67031746 | 67032151 |
| chr21 | 10514893 | 10515878 | chr4 | 137528851 | 137530683 | chr10 | 95718667 | 95719694 |
| chr18 | 12306466 | 12306788 | chr6 | 17222864 | 17223271 | chr15 | 66095859 | 66096408 |
| chr11 | 11678106 | 11679220 | chr8 | 596282 | 596967 | chr10 | 126798689 | 126798989 |
| chr7 | 143511313 | 143511651 | chr8 | 47763417 | 47764319 | chr14 | 69572928 | 69573560 |
| chr18 | 13395461 | 13396085 | chr2 | 624552 | 625503 | chr2 | 119690441 | 119691192 |
| chr1 | 245528140 | 245528445 | chr13 | 23866042 | 23866697 | chr19 | 15732061 | 15734116 |
| chr22 | 45551705 | 45552606 | chr2 | 11498499 | 11500081 | chr20 | 62736856 | 62737327 |
| chr18 | 8718293 | 8718695 | chr16 | 87490095 | 87491028 | chr3 | 9833138 | 9833796 |
| chr22 | 36741909 | 36743012 | chr20 | 30892526 | 30893033 | chr1 | 113146646 | 113148014 |
| chr19 | 55372019 | 55372593 | chr22 | 24551105 | 24552271 | chr10 | 45599753 | 45601265 |
| chr12 | 34361022 | 34361367 | chr11 | 134445762 | 134446328 | chr1 | 227558330 | 227558660 |
| chr3 | 142947186 | 142947702 | chr1 | 93697342 | 93697871 | chr12 | 121101452 | 121102937 |
| chr10 | 44879581 | 44879900 | chr1 | 91546988 | 91547264 | chr11 | 117563766 | 117564474 |
| chr19 | 7698740 | 7699254 | chr14 | 104108260 | 104108592 | chr1 | 147077720 | 147079103 |
| chr6 | 27335259 | 27336946 | chr10 | 120011563 | 120011956 | chr20 | 59788261 | 59789086 |
| chr7 | 6170202 | 6170683 | chr9 | 135152772 | 135153288 | chr17 | 13445938 | 13449473 |
| chr16 | 57904035 | 57904553 | chr21 | 39989569 | 39991486 | chr22 | 48049142 | 48049888 |
| chr17 | 35274107 | 35275863 | chr4 | 184039963 | 184040179 | chr7 | 156942826 | 156942970 |
| chr7 | 61047578 | 61048416 | chr11 | 8203956 | 8205093 | chr21 | 8208513 | 8208637 |
| chr10 | 37680537 | 37681128 | chr10 | 117790628 | 117791732 | chr12 | 8226610 | 8227622 |
| chr18 | 14146538 | 14146871 | chr1 | 50024419 | 50025034 | chr11 | 44845206 | 44846379 |
| chr2 | 149500167 | 149502836 | chr6 | 41416320 | 41417665 | chr2 | 20669547 | 20669882 |
| chr4 | 85354617 | 85355300 | chr16 | 13420883 | 13422890 | chr17 | 81415254 | 81415703 |
| chr16 | 86818684 | 86819387 | chr9 | 95517620 | 95517989 | chr4 | 11368618 | 11368785 |
| chr5 | 157112690 | 157113813 | chr16 | 89957317 | 89958043 | chr21 | 8987447 | 8987564 |
| chr7 | 28504497 | 28505240 | chr1 | 3684171 | 3684356 | chr2 | 20671271 | 20671519 |
| chr19 | 44633656 | 44634613 | chr12 | 1849981 | 1851423 | chr17 | 14294990 | 14295958 |
| chr7 | 150737823 | 150738721 | chr19 | 3124354 | 3125226 | chr6 | 157639669 | 157640074 |
| chr1 | 19371220 | 19371613 | chr17 | 79614522 | 79615627 | chr13 | 25385975 | 25386793 |
| chr21 | 44353732 | 44354310 | chr11 | 119526755 | 119527380 | chr4 | 8044820 | 8045180 |
| chr19 | 4748218 | 4748842 | chr16 | 16166162 | 16166650 | chr1 | 112742887 | 112743330 |
| chr16 | 32202671 | 32202805 | chr1 | 7702455 | 7703316 | chr19 | 33684941 | 33685467 |
| chr19 | 49133596 | 49134204 | chr8 | 130518515 | 130518789 | chr11 | 45319323 | 45321325 |
| chr16 | 88478081 | 88478643 | chr22 | 46061050 | 46061505 | chr7 | 42522398 | 42522960 |
| chr15 | 25693702 | 25694577 | chr10 | 3414489 | 3415171 | chr13 | 111286458 | 111287121 |
| chr5 | 534024 | 534383 | chr11 | 45649836 | 45650466 | chr7 | 3187535 | 3187826 |
| chr22 | 37340611 | 37340899 | chr7 | 158984608 | 158985860 | chr16 | 66921440 | 66922164 |
| chr19 | 45396819 | 45398059 | chr20 | 18504430 | 18504726 | chr16 | 14989486 | 14990283 |
| chr4 | 1001149 | 1001739 | chr9 | 95104047 | 95104626 | chr7 | 58112098 | 58112679 |
| chr6 | 342323 | 342894 | chr10 | 104630945 | 104631324 | chr7 | 91265917 | 91266859 |
| chr19 | 28865373 | 28866707 | chr6 | 31118398 | 31119821 | chr10 | 131313580 | 131314153 |
| chr1 | 227798382 | 227798774 | chr19 | 50217044 | 50217624 | chr1 | 1438166 | 1439280 |
| chr2 | 11540112 | 11540916 | chr20 | 63246529 | 63246860 | chr1 | 112741866 | 112742605 |
| chr17 | 72487693 | 72488293 | chr19 | 3457178 | 3457688 | chr12 | 128725015 | 128725575 |
| chr10 | 86374199 | 86375546 | chr1 | 23889627 | 23891160 | chr14 | 76330270 | 76330660 |
| chr12 | 132216395 | 132216797 | chr13 | 24340143 | 24340883 | chr2 | 232386451 | 232387541 |
| chr16 | 5850991 | 5851720 | chr14 | 68628619 | 68629095 | chr8 | 132278934 | 132280552 |
| chr20 | 63819497 | 63819987 | chr10 | 1362887 | 1364024 | chr2 | 1813491 | 1813799 |
| chr16 | 84839721 | 84840285 | chr17 | 34424329 | 34425004 | chr11 | 20310247 | 20311130 |
| chr6 | 168939447 | 168939951 | chr12 | 119069217 | 119070006 | chr7 | 128916087 | 128916595 |
| chr17 | 57050055 | 57052372 | chr5 | 57405365 | 57408472 | chr10 | 102240418 | 102240645 |
| chr21 | 8212342 | 8212511 | chr13 | 24327897 | 24328557 | chr12 | 130390430 | 130391638 |
| chr14 | 87733786 | 87734176 | chr17 | 83077947 | 83078499 | chr4 | 8686080 | 8687624 |
| chr19 | 2501724 | 2502477 | chr4 | 131975207 | 131976212 | chr10 | 77108002 | 77108555 |
| chr15 | 73951689 | 73952154 | chr14 | 99191169 | 99192305 | chr14 | 20723146 | 20723917 |
| chr15 | 76334390 | 76334853 | chr17 | 42544643 | 42545424 | chr7 | 98034517 | 98034664 |
| chr16 | 27945359 | 27945658 | chr12 | 132392879 | 132393412 | chr2 | 19669280 | 19670595 |
| chr1 | 46453595 | 46454487 | chr12 | 54077627 | 54078184 | chr1 | 30377635 | 30378581 |
| chr19 | 3306713 | 3307185 | chr22 | 42318237 | 42319418 | chr4 | 180437995 | 180439658 |
| chr16 | 25723851 | 25724787 | chr7 | 158957515 | 158958085 | chr5 | 16372144 | 16373958 |
| chr16 | 596136 | 596550 | chr14 | 20517953 | 20519601 | chr3 | 126660001 | 126660631 |
| chr19 | 50972000 | 50972643 | chr15 | 98776829 | 98777534 | chr11 | 82943585 | 82946622 |
| chr20 | 30723551 | 30723817 | chr8 | 2000221 | 2000510 | chr2 | 39242960 | 39244106 |
| chr19 | 48768112 | 48769139 | chr17 | 4878352 | 4880043 | chr14 | 106504877 | 106505281 |
| chr17 | 58062614 | 58063959 | chr19 | 39891802 | 39892086 | chr5 | 132822960 | 132823511 |
| chr9 | 4118203 | 4118593 | chr2 | 119129919 | 119130738 | chr5 | 178383792 | 178384431 |
| chr16 | 768252 | 768863 | chr10 | 484621 | 485566 | chr3 | 49910431 | 49910983 |
| chr19 | 50450857 | 50451120 | chr1 | 12539422 | 12540382 | chr6 | 69600552 | 69601322 |
| chr3 | 61829034 | 61829597 | chr16 | 86778274 | 86778971 | chr8 | 104367339 | 104367780 |
| chr6 | 112367141 | 112367475 | chr5 | 179472459 | 179473058 | chr3 | 72928866 | 72929141 |
| chr12 | 130555379 | 130556137 | chr20 | 37862529 | 37864451 | chr2 | 30782110 | 30782755 |
| chr2 | 24848557 | 24849094 | chr6 | 165682380 | 165683868 | chr11 | 2890696 | 2891150 |
| chr11 | 107981155 | 107982175 | chr19 | 1789508 | 1789803 | chr20 | 62218440 | 62218734 |
| chr7 | 62446753 | 62447942 | chr15 | 27214322 | 27216136 | chr19 | 22709251 | 22709459 |
| chr16 | 817543 | 818558 | chr17 | 39813863 | 39813997 | chr16 | 86593936 | 86594453 |
| chr12 | 116434380 | 116435007 | chr19 | 7864141 | 7864448 | chr1 | 2463381 | 2463659 |
| chr10 | 117720231 | 117721341 | chr1 | 90184479 | 90187416 | chr19 | 474608 | 474917 |
| chr2 | 238907563 | 238909805 | chr8 | 26488563 | 26489090 | chr17 | 6497972 | 6498428 |
| chr1 | 242212381 | 242212726 | chr3 | 64443610 | 64444257 | chr22 | 49266520 | 49266666 |
| chr14 | 76728823 | 76731142 | chr18 | 76787629 | 76787965 | chr2 | 23563322 | 23563770 |
| chr6 | 168794377 | 168794851 | chr12 | 54021238 | 54021859 | chr12 | 129696560 | 129697154 |
| chr12 | 48468830 | 48470338 | chr10 | 130906777 | 130907216 | chr17 | 68591334 | 68591939 |
| chr8 | 142639643 | 142641630 | chr21 | 42642453 | 42642994 | chr11 | 116732288 | 116732466 |
| chr19 | 45914342 | 45914812 | chr9 | 63813181 | 63813858 | chr7 | 26526125 | 26526658 |

TABLE 3
(all positions of the regions/features are described herein in reference to the numbering of
the human reference genome GRCh38 with the GenBank assembly accession code GCA_000001405.15)

Region

| | | | |
|---|---|---|---|
| chr1: 117570557-117572097 | chr12: 119179322-119181333 | chr17: 5592781-5593637 | chr21: 32432920-32433489 |
| chr1: 119003772-119004906 | chr12: 130450583-130452531 | chr17: 74194490-74195058 | chr21: 32432920-32433489 |
| chr1: 173703822-173706101 | chr12: 130450633-130452613 | chr17: 75940049-75942832 | chr21: 32834344-32835558 |
| chr1: 211511580-211513970 | chr12: 131101663-131102321 | chr17: 75940049-75942832 | chr21: 38713920-38715584 |
| chr1: 231495885-231496270 | chr12: 131207455-131208071 | chr17: 787922-789719 | chr21: 38727691-38728795 |
| chr1: 234379519-234380957 | chr12: 132322751-132323486 | chr17: 8116594-8117335 | chr21: 39569521-39570535 |
| chr1: 2825914-2826582 | chr12: 15349973-15352575 | chr18: 39006173-39007262 | chr21: 43985999-43986828 |
| chr1: 2968467-2968793 | chr12: 57283464-57283977 | chr19: 12160225-12160875 | chr22: 20205706-20206011 |
| chr1: 3268196-3268501 | chr13: 106392893-106394316 | chr19: 1923049-1924860 | chr22: 27554231-27555215 |
| chr1: 3690410-3690958 | chr13: 112229788-112230545 | chr19: 38082519-38083138 | chr22: 31110864-31112881 |
| chr1: 38029231-38029807 | chr13: 112980999-112981650 | chr19: 4293801-4294550 | chr22: 44905155-44905830 |
| chr10: 101200108-101200568 | chr13: 26872420-26873040 | chr19: 455414-455675 | chr22: 48053558-48054278 |
| chr10: 102967031-102967923 | chr13: 61364476-61367725 | chr19: 50722370-50723201 | chr3: 101540886-101542127 |
| chr10: 103927517-103928489 | chr13: 91399570-91400232 | chr19: 50758003-50758727 | chr3: 122660626-122661115 |
| chr10: 114075797-114076554 | chr14: 101501279-101501779 | chr19: 5484577-5485079 | chr3: 125823813-125826797 |
| chr10: 11439-11502 | chr14: 101570051-101572986 | chr19: 55958551-55959035 | chr3: 133596504-133598682 |
| chr10: 120194989-120198322 | chr14: 105707267-105707945 | chr19: 6669960-6670777 | chr3: 14216290-14217120 |
| chr10: 122124374-122125529 | chr14: 36804555-36808056 | chr19: 974444-975102 | chr3: 185399055-185400609 |
| chr10: 128749569-128752029 | chr14: 89916636-89920924 | chr2: 101464840-101465903 | chr3: 197022584-197024699 |
| chr10: 131688983-131689466 | chr14: 96653314-96654735 | chr2: 105619604-105620178 | chr3: 50470455-50471273 |
| chr10: 14031725-14032854 | chr15: 27587106-27589228 | chr2: 232419540-232420315 | chr4: 16528775-16530218 |
| chr10: 1598935-1600040 | chr15: 29871222-29871742 | chr2: 234421976-234424374 | chr4: 172831735-172832945 |
| chr10: 26913471-26914759 | chr15: 33200444-33201430 | chr2: 239832738-239833236 | chr4: 179397136-179398578 |
| chr10: 26970597-26972886 | chr15: 38554159-38554832 | chr2: 240213376-240213867 | chr4: 185847492-185848171 |
| chr10: 31585878-31586459 | chr15: 40612399-40612796 | chr2: 240291404-240292182 | chr4: 186294245-186295961 |
| chr10: 42103229-42103950 | chr15: 71129763-71130407 | chr2: 240519761-240520278 | chr4: 3732763-3733364 |
| chr10: 46050651-46051742 | chr16: 12188920-12190286 | chr2: 241774335-241775375 | chr4: 8657247-8657748 |
| chr10: 46053939-46056094 | chr16: 24450604-24452238 | chr2: 47728769-47730252 | chr5: 132100633-132101523 |
| chr10: 79120324-79121388 | chr16: 3836106-3837548 | chr2: 88765920-88766350 | chr5: 152106798-152110177 |
| chr10: 98025750-98026917 | chr16: 49670218-49670981 | chr2: 89585751-89586303 | chr5: 157505763-157507175 |
| chr11: 114614996-114615981 | chr16: 65951478-65954990 | chr2: 89767489-89768080 | chr5: 176571681-176572812 |
| chr11: 117198968-117199615 | chr17: 10123801-10124372 | chr2: 9738884-9739863 | chr5: 177955263-177955834 |
| chr11: 120882925-120883807 | chr17: 18201494-18202088 | chr20: 1778535-1779145 | chr5: 179852110-179853570 |
| chr11: 125129418-125130067 | chr17: 20030958-20031901 | chr20: 24034206-24035063 | chr5: 1827708-1828541 |
| chr11: 1563304-1564048 | chr17: 21693120-21693636 | chr20: 24970201-24973927 | chr5: 2415523-2416200 |
| chr11: 1876829-1877810 | chr17: 28414759-28416583 | chr20: 2779445-2780925 | chr5: 35531145-35531768 |
| chr11: 68649691-68650284 | chr17: 29187169-29187897 | chr20: 56702185-56702980 | chr5: 71807506-71810299 |
| chr12: 109201559-109201843 | chr17: 39707511-39707828 | chr20: 57984057-57985396 | chr6: 134120667-134122250 |
| chr12: 112854889-112856379 | chr17: 47813565-47813964 | chr20: 64052442-64052862 | chr6: 135162968-135164612 |
| chr9: 133948144-133948772 | chr7: 20735578-20737371 | chr8: 37724816-37726067 | chr6: 151808909-151809540 |
| chr9: 134809726-134810418 | chr7: 2985064-2986066 | chr8: 58799244-58803560 | chr6: 161835293-161835833 |
| chr9: 135302974-135303333 | chr7: 39833863-39834291 | chr8: 64798980-64799609 | chr6: 167794851-167795750 |
| chr9: 32383101-32383771 | chr7: 6622257-6623328 | chr8: 69832174-69832473 | chr6: 169654368-169655364 |
| chr9: 35406223-35407017 | chr7: 72447027-72447547 | chr8: 87868828-87869743 | chr6: 26017152-26019604 |
| chr9: 62844696-62845119 | chr7: 98545976-98547156 | chr8: 94168929-94170967 | chr6: 30371543-30375792 |
| chr9: 68318186-68319030 | chr8: 142608435-142608773 | chr8: 96021404-96024029 | chr6: 30418022-30420417 |
| chr9: 72044862-72047642 | chr8: 20458906-20459949 | chr9: 109745111-109746929 | chr6: 37648161-37650402 |
| chr9: 7214678-7217189 | chr8: 22550487-22551167 | chr9: 113564372-113565631 | chr6: 41527393-41528420 |
| chr9: 85884199-85884776 | chr8: 30971189-30974376 | chr9: 114610029-114610695 | chr6: 57274504-57275060 |
| chr9: 93039152-93040478 | chr9: 122131291-122132118 | chr9: 115476801-115478830 | chr6: 8277473-8279189 |
| chr9: 93905637-93906609 | chr9: 130326446-130326834 | chr9: 121990487-121991481 | chr6: 99352108-99354683 |
| chr7: 150923150-150924719 | chr9: 132805098-132806220 | chr7: 130140414-130140996 | chr7: 100825889-100826539 |
| chr7: 155405454-155405926 | chr9: 132854175-132854666 | | |

### Example 2: Use of the Methylation Signature in the Clinical Settings as IVD

### Protocol #1

1. Draw 20 mL blood from subject
2. Separate Plasma by traditional centrifugation steps
3. Purify cfDNA (10 ng-1 ug) from plasma sample using molecular biology techniques such as column-based purification of nucleic acids.
4. cfDNA quality assessment using dye-based by fluorescence assays
5. Bisulfite Conversion of cfDNA (e.g. using e Zymo EZ DNA Methylation Lightning Kit)
6. Purification of the bisulfite-treated DNA on a spin column
7. Preparation of sequencing library (e.g. using the EpiGnome™ Kit by Epicentre)
8. Sequencing of the library (e.g. using HiSeq 2500 System) to determine methylation status of the genes identified in Example 1
9. Creation of sequencing files (e.g. FASTQ files)
10. Annotation of sequenced methylated DNA on human reference genome (e.g. bisulfite-converted UCSC HG19 reference genome)
11. Statistical analysis to determine differential methylation calculations.
12. Use of computational methods and/or machine learning techniques to determine diagnosis of AD condition

### Example 3

### Project Population, Inclusion and Exclusion Criteria

Patients with Clinically Diagnosed AD

### Inclusion Criteria:

Clinical diagnosis of AD
Aged 40 years old or over
Probable AD dementia and with evidence of an AD pathophysiological process based on international guidelines (McKhann et al., 201116) or Mild Cognitive Impairment (MCI) due to AD based on international guidelines (Albert 201117).

### Healthy Age- and Gender-Matched Subjects

### Inclusion Criteria:

Healthy subject
Age- and gender-matched with the AD patient population
No known disease
Preserved cognitive capacity

### Exclusion Criteria for Both Populations:

Other neurodegenerarive disorders or cause of cognitive decline
Subjects at risk for blood draw

### Sample Collection

Blood samples will be collected in 5×10 mL (50 ml) PAXgene Blood ccfDNA Tubes, Qiagen. cfDNA will next be purified from plasma using QlAamp Circulating Nucleic Acid Kit and its quantity determined by fluorescence-based commercial kits.

Analysis of ccfDNA
Quality Control of ccfDNA
Amount of the cfDNA extracted from plasma to be used: 10 ng to 50 ng of ctDNA
Microelectrophoresis profile on Fragment Analyzer™

### Go/NoGo

Quality control should show the typical nucleosome peak at 166 bp

ccfDNA Library Preparation

Library preparation using New England Biolabs® Ultra II kit—Specific enzymatic conversion using NEBNext® Enzymatic Methyl-seq (including provided spikes for methylation conversion rate calculation)—Unique double-indexing of the libraires (UDI) Monitoring of the PCR amplification

### Quality Control

Dosage by qPCR and microelectrophoresis profile on Fragment Analyzer™

### Human Genome High Throughput Sequencing

Libraries are sequenced on full S4 flow cells (corresponding to 3×flow cells for the 50 samples) on Illumina® NovaSeqTM6000 using 2×100 bases paired end mode to obtain paired-end 150 bp reads aiming at 30× coverage for the whole genome.

### Bioinformatic

Extensive quality control of the sequences, such as sample demultiplexing and/or trimming, allowed the creation of FASTQ sequence files and FASTQC files for the subsequent analysis.

### Differential Methylation Analysis

Raw sequencing data in FastQ file format were aligned to the human genome (GRCh38 version) using the Bismark software (version 0.23.1). The software was run using default parameters except for the following parameters: —L 28—D 3—R 0. Using the deduplicate_bismark command (default parameters), alignments to the same position in the genome from the Bismark mapping output, which can arise by PCR amplification were removed. In the next step we used bismark_methylation_extractor to extracts the methylation call for every single C analysed. The following arguments were set: —no_overlap—comprehensive—merge_non_CpG.

Evaluation of differences in methylation status between cases and controls was performed using the dmrseq package (version 1.16.0) for R/Bioconductor (version 4.1) environment. CpG coverage and methylation status data, stored in the “*.bismark.cov.gz”, one file per patient, were imported and merged using read.bismark( ) function. CpGs were filtered selecting 19715509 loci with a coverage >=8× in all 50 samples.

From the study cohort, a training set (80%, 24 cases and 16 controls) and a validation set (20% 6 cases and 4 controls) were identified. Differential analysis was performed on the training set using the dmrseq( ) function. Default parameters were used expect for: cutoff=0.02, minNumRegion=20, maxPerms=50. 9149 regions were identified genome wide with 14 having a p-value<0.001. Those regions became the input for the training of a classifier able to accurately discriminate between cases and controls using the most relevant subset of CpG regions. We used a regularized logistic regression model as implemented in the glmnet R/Bioconductor package (version 4.1-4). All 9149 regions previously identified in 40 samples were used as candidate features. By varying the penalization parameter lambda between 10-15 and 1, the optimal subset of features required was selected, by optimizing the prediction performance (measured as area under the ROC curve or AUC) in a 4-fold cross-validation setting. We used the cv.glmnet( ) function, additionally setting: alpha=0.5, keep=T, grouped=FALSE. The fully trained model was applied to the 10 samples of the independent validation set not used for the development of the classifier. The whole cross-validation procedure was repeated 100 times to quantify the variability caused by the sampling procedure. For each iteration, the optimal number of features included (median=274), the cross-validated AUC (median=0.99) and the AUC for the independent validation set (median=0.83) were collected.

### Example 4

The microarray contains probes for the detection of the genes/regions selected from tables 1, 2, and/or 3 and additional genomic regions (e.g., as negative controls in number equal to the ones for table 1, 2 and 3). The microarray can be a customized array or a commercially available one such as the Illumina Infinium MethylationEPIC BeadChip arrays (Catalog No. WG-317-1003) for methylation profiling.

Purified cfDNA will be subjected to methylation (either bisulfite or enzymatic) conversion. Following methylation conversion, methylation profiling will be obtained by incubating the microarray with converted cfDNA and imaging it on specific devices such as Illumina iScan System (Illumina, Inc., USA).

Obtained microarray results are compared to a machine learning model (trained on prelabelled data as described herein) and classified based on this comparison to obtain a score of AD diagnosis.

### Example 5

A subject shows symptoms that are common among different types of dementia-related disorders. The methylation status of cell-free DNA obtained from fresh or frozen plasma is analysed and the results are compared to a machine learning model (trained on prelabelled data as described herein) and classified based on this comparison to discriminate between AD or non-AD dementias.

1. A method for determining a score indicative of the diagnosis of a neurodegenerative disease or disorder of a subject, of the probability of a subject to develop a neurodegenerative disease or disorder and/or of the disease progression of a neurodegenerative disease or disorder, the method comprising the steps of:
a) obtaining at least one methylation status of two or more genes selected from Table 1 and/or two or more regions selected from Table 2 from a sample of a subject;
b) comparing the methylation status obtained in (a) to a reference pattern; and
c) determining a score indicative of the diagnosis of a neurodegenerative disease or disorder of the subject, of the probability of the subject to develop a neurodegenerative disease or disorder and/or of the disease progression of a neurodegenerative disease or disorder based on the comparison obtained in (b).

2. The method of claim 1, wherein the sample is a body fluid sample preferably a plasma or serum sample, more preferably a plasma sample.

3. The method of claim 2, wherein the sample was frozen.

4. The method of claim 1, wherein the methylation status is/are obtained from cell free-DNA in the sample.

5. The method of claim 1, wherein obtaining at least one methylation status comprises genome methylation profiling.

6. The method of claim 1, wherein the at least one methylation status comprises the methylation status of at least 100 regions selected from Table 2, preferably at least 150 regions selected from Table 2, preferably at least 200 regions selected from Table 2, more preferably between 200 and 600 regions selected from Table 2, more preferably 250 and 500, more preferably between 250 and 300 regions selected from Table 2.

7. The method of claim 6, wherein the at least one methylation status comprises the methylation status of at least 80% of the regions selected from Table 3, preferably 90% of the regions selected from Table 3, more preferably all regions selected from Table 3.

8. The method of claim 1, wherein the methylation status is determined in regions with
a) an average nucleotide width of less than 5000, preferably less than 4000, more preferably less than 3000, more preferably less than 2000, more preferably less than 1000; and/or
b) a median nucleotide width of less than 5000, preferably less than 4000, more preferably less than 3000, more preferably less than 2000, more preferably less than 1000.

9. The method of claim 1, wherein at least one further marker is obtained in (a) compared in (b).

10. The method of claim 9, wherein the at least one further marker is a marker selected from the group of subject background data, cognitive performance marker, autonomic nervous system biomarker.

11. The method of claim 1, wherein the reference pattern is obtained from the methylation status of two or more genes selected from Table 1 and/or two or more regions selected from Table 2 from samples of at least two reference subjects, wherein at least one of the reference subjects suffers from a neurodegenerative disease or disorder.

12. The method of claim 10, wherein obtaining the reference pattern from the methylation status of two or more genes selected from Table 1 and/or two or more regions selected from Table 2 from samples of at least two reference subjects comprises a machine learning technique.

13. A method for monitoring a neurodegenerative disease or disorder, the method comprising the steps of:
i) determining a first score indicative of the probability of a subject to develop a neurodegenerative disease or disorder and/or of the disease progression of a neurodegenerative disease or disorder according to the method of claim 1 at a first timepoint;
ii) determining a second score indicative of the probability of a subject to develop a neurodegenerative disease or disorder and/or of the disease progression of a neurodegenerative disease or disorder according to said method at a second timepoint;
iii) comparing the first score of step (i) with the second score of step (ii); and iv) monitoring the disease progression of the neurodegenerative disease or disorder in the subject based on the comparison of step (iii).

14. A library comprising at least one score indicative of the probability of a subject to develop a neurodegenerative disease or disorder and/or of the disease progression of a neurodegenerative disease or disorder determined according to claim 1.

15. A storage device comprising computer-readable program instructions to execute the method according to claim 1.

16. A server comprising the storage device of claim 15, at least one processing device, and a network connection for receiving data indicative of at least one methylation status.

17. The method of claim 1, wherein the neurodegenerative disease or disorder is a disease or disorder characterized by cognitive impairment.

18. The method of claim 1, wherein the neurodegenerative disease or disorder is Alzheimer's disease and/or Parkinson's disease.

19. The method of claim 18, wherein the neurodegenerative disease or disorder is Alzheimer's disease.

20. A device comprising methylation specific oligonucleotide probes, wherein the probes are specific for the determination of the methylation status of at least 80% of the regions selected from Table 3, preferably all regions selected from Table 3.

21. The device according to claim 20, wherein the device is a microarray.

22. A method for
a) determining a score indicative of the diagnosis of a neurodegenerative disease or disorder of a subject, of the probability of a subject to develop a neurodegenerative disease or disorder and/or of the disease progression of a neurodegenerative disease or disorder; and/or
b) monitoring a neurodegenerative disease or disorder, using the device of claim 20.

23. (canceled)

24. A method for determining a score indicative of the diagnosis of a neurodegenerative disease or disorder of a subject, of the probability of a subject to develop a neurodegenerative disease or disorder and/or of the disease progression of a neurodegenerative disease or disorder, the method comprising the steps of:
a) obtaining at least one methylation status of two or more genes selected from Table 1 and/or two or more regions selected from Table 2 from a sample of a subject, using the device of claim 20;
b) comparing the methylation status obtained in (a) to a reference pattern; and
c) determining a score indicative of the diagnosis of a neurodegenerative disease or disorder of the subject, of the probability of the subject to develop a neurodegenerative disease or disorder and/or of the disease progression of a neurodegenerative disease or disorder based on the comparison obtained in (b).
